(19) United States
(12) Patent Application Publication
(10) Pub. No.: US 2025/0051407 A1
(43) Pub. Date: Feb. 13, 2025

(54) De novo designed sequence-specific DNA binding proteins

(71) Applicant: University of Washington, Seattle, WA (US)

(72) Inventors: David Baker, Seattle, WA (US); Cameron Glasscock, Seattle, WA (US); Robert Pecoraro, Seattle, WA (US); Ryan McHugh, Seattle, WA (US); Christoffer Norn, Seattle, WA (US); Frank DiMaio, Seattle, WA (US); David Benjamin Turitz Cox, Seattle, WA (US); Brian Coventry, Seattle, WA (US); Hugh Haddox, Seattle, WA (US); Gyu Rie Lee, Seattle, WA (US)

(21) Appl. No.: 18/799,107

(22) Filed: Aug. 9, 2024

Related U.S. Application Data
(60) Provisional application No. 63/532,229, filed on Aug. 11, 2023.

(51) Int. Cl. C07K 14/47; C12N 15/70

(52) U.S. Cl. CPC C07K 14/4702; C12N 15/70; C07K 2319/71; C07K 2319/80

(57) ABSTRACT

Polypeptides are provided that include an amino acid sequence at least 50% identical, not including any amino acid insertions at identified insertion sites, to the amino acid sequence selected from the group consisting of SEQ ID NO: 1-52, wherein the polypeptide is a sequence-specific DNA-binding polypeptide, fusion proteins comprising such polypeptides, nucleic acids encoding such polypeptides and fusion proteins, and expression vectors and host cells comprising such nucleic acids.

### FEDERAL FUNDING STATEMENT

This invention was made with government support under Grant No. MFB 2226466, awarded by the National Science Foundation. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Aug. 7, 2024 having the file name “24-0983-US” and is 65,656 bytes in size.

### BACKGROUND

DNA-binding proteins (DBPs) are critical for molecular biology, gene regulation, genome engineering, therapeutics, and diagnostics. As such, extensive efforts have been made over decades to develop programmable DBP systems that target specific DNA sequences, notably Cys2His2 zinc finger (ZF) domains, transcription activator-like effectors (TALEs), and CRISPR-Cas. Each approach has significant limitations: ZFs can be laborious to engineer, and the size of TALE and CRISPR-Cas systems complicates their delivery in therapeutic applications; CRISPR-Cas systems also require an extra guide RNA component and target sites are constrained by protospacer adjacent motif (PAM) requirements. Computational approaches for DBP engineering have been limited to redesigning interfaces of existing native protein-DNA complex structures. These efforts have been constrained by the rigid geometry of the starting scaffold shape and orientation relative to DNA, which restricts the possible target sequences that can be recognized.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical, not including any amino acid insertions at identified insertion sites (i.e., any insertions are not considered when determining percent identity to the reference polypeptide), to the amino acid sequence selected from the group consisting of SEQ ID NO:1-52, wherein the polypeptide is a sequence-specific DNA-binding polypeptide. In some embodiments, residues in bold font are conserved (i.e., identical) relative to the reference sequence. In other embodiments, underlined residues are conserved relative to the reference sequence.

In one embodiment, the polypeptides comprises an amino acid sequence at least 50% identical to the amino acid sequence selected from the group consisting of SEQ ID NO:1, 2, 4, 15, 21-23, 25, 26, 30, 31, 34, 36, 38, 44, and 49-52. In a further embodiment, substitutions relative to the reference sequence are selected from residues listed in columns 2 or 3 of one of Tables 4-19. In another embodiment, only conservative substitutions, or no substitutions are permitted relative to the reference sequence at interface residues, and/or at core residues identified in Table 4-19. In another embodiment, substitutions relative to the reference sequence are conservative amino acid substitutions.

In one embodiment, the disclosure provides fusion proteins, comprising (a) the polypeptide of any embodiment or combination of embodiments herein; and (b) one or more functional domains. In a further embodiment, the one or more functional domains is selected from the group consisting of a transcriptional effector domain, a multimerization scaffold protein, a nucleotide editing domain, a DNA methyltransferase domain, a nickase domain, a recombinase/integrase domain and a nuclease.

In another embodiment, the disclosure provides nucleic acid encoding the polypeptide or fusion protein of any embodiment or combination of embodiments herein. The disclosure further provides expression vectors comprising the nucleic acid of any embodiment herein operatively linked to a promoter, and host cells comprising the polypeptide, fusion protein, nucleic acid, and/or expression vector of any embodiment herein.

In one embodiment, the disclosure provides kits comprising:
(a) a first expression vector comprising the nucleic acid of any embodiment herein operatively linked to a promoter; and
(b) a second expression vector comprising a DNA target of the polypeptide expressed by the first expression vector.

In another embodiment, the disclosure provides kits comprising:
(a) a first host cell comprising a chromosomally-integrated expression cassete comprising the nucleic acid of any embodiment or combination of embodiments herein, operatively linked to a promoter; and
(b) a second host cell comprising a chromosomally-integrated DNA target of the polypeptide expressed by the expression cassette.

### DESCRIPTION OF THE FIGURES

FIG. 1. Designed DBPs bind with high affinity and specificity to their intended target sites. A-E, Characterization of DBPs 6, 35, 48, 56, and 62, respectively. Left, Computational design models of characterized designs at the DNA-binding interface. DNA bases and protein residues involved in hydrogen bonding interactions are shown. Hydrogen bonds are highlighted with dashed lines. Middle, Relative binding activity (PE/FITC normalized to the no-competitor condition) from flow cytometry analysis in yeast display competition assays with all possible DNA base mutations at each position of the competitor oligo. Competitor mutations where competition was weak suggest incompatibility with binding to the competitor oligo. Arrows indicate base positions contacted with hydrogen bonds or hydrophobic contacts to base atoms in the design model. DBP48 was analyzed with sequence D due to its improved binding signal and nearly identical modeled binding sites; all other designs were analyzed with their designed target sequence. Additional characterized designs are shown in FIG. 5. Right, Binding of purified miniprotein designs to the DNA target with BLI. Each line represents biotinylated dsDNA target dilutions by 1%3. The highest DNA target concentration is indicated in each plot. Additional characterized designs are shown in FIG. 6. DBP48 was analyzed by BLI for binding using the sequence D dsDNA target. F, DBPs 6 and 48 differ in both structure and docking mode to native co-complex structures with matching DNA binding sites). G, DBP35 has similar structure and dock to the closest match in the PDB, but binds a distinct DNA target site, while DBPs 56 and 62 have structures quite similar to the closest matches in the PDB but different docks and DNA target sites.

FIG. 2. Designed DBPs function in living cells to direct transcriptional repression and activation. A, Transcriptional repression in Escherichia coli. Vectors encoding the DBP-TetR repressor variants were constructed with a repressor under control of the IPTG-inducible P_{Tac} promoter. A synthetic promoter containing the designed DBP binding sites around the −10 and −35 elements was used to control expression of YFP. B, Normalized median YFP Fluorescence from flow cytometry analysis of cells containing the successful DBP57-TetR (upper (SEQ ID NO: 62)) and DBP48-TetR (lower (SEQ ID NO: 63)) NOT gate circuits. Fold repression of YFP was ˜8.1× and ˜3.4× for cognate DBP57-TetR (upper left) and DBP48-TetR (lower right) circuits, respectively, upon induction with 1 mM IPTG; however, minimal repression was observed when variants were encoded with the swapped synthetic promoters (upper right, lower left). C, Transcriptional activation in HEK293T cells. ENGRAM was used to measure the activities of the synTFs. synTFs were created by fusing GCN4 dimerization domain and VP64 activation domain to the C-termini of the DBPs. The synTF-specific cis-regulatory elements (CRE) were created by evenly distributing palindromic binding motifs on a 130 bp transcriptionally inactive DNA sequence where each CRE drives a uniquely barcoded pegRNA. Once activated, the barcode can be recorded into DNA TAPE (HEK3 locus in the genome). D, Fold activation of synTFs measured as normalized barcode abundance in the DNA TAPE demonstrates successful activation of the recorders. DBP9/DBP35opt and DBP57/DBP60 showed 3-5 fold activation dependent on the spacing between motifs, while DBP48 didn't show detectable activation.

FIG. 3. Clonal analysis of binder designs by yeast surface display confirms dsDNA-binding function. A, Histograms of binding activity (PE/FITC) are shown for each design. Knockout sequences were created by mutating 1-3 key interface residues for base-specific contacts present in the wildtype (WT) design model (table 1). Samples of the WT design (WT+DNA), and the knockout sequence (KO+DNA) with target were analyzed after labeling with each respective dsDNA oligo at 1 μM with avidity (DBPs 7, 10, 11, 24, 25, 26, 28, 31, and 40 collected without avidity). The background signal of the wildtype design without dsDNA labeling (WT-DNA) is shown. Interface knockouts substantially disrupted dsDNA-binding in nearly all cases.

FIG. 4. All-by-all analysis of selected designs by yeast surface display reveals preferential target binding of designs. A, Yeast surface display relative binding activity (Normalized PE/FITC) of each design labeled at 1 μM dsDNA with avidity, normalized by design row. Squares indicate the intended target sequence for each design. Dots indicate target sequences containing Rosetta™-predicted binding motifs. Sequences were considered potential binding targets if they had Rosetta™ ΔΔG less than or equal to the designed complex. DBPs 83, 85, 65, 6, 9, 35, 69, 47, 48, 51, 56, 57, 60, and 62 were considered to preferentially bind less than 3 of the 13 tested DNA target sequences, including their designed target sequence. B, DBP90 bound weakly to its initial design target, but strongly to an alternate target sequence (H) with slightly higher Rosetta™ ΔΔG but also allowed for bidentate hydrogen bonds to 3 bases. Left: DBP90+A (on-target model), Right DBP90+H (alternate target model). C, DBP48 bound weakly to its initially designed target sequence, but strongly to Rosetta™-predicted alternative target site (D) that differed by only 1 base pair across the interface and had equivalent Rosetta™ ΔΔG. Left: DBP48+I (on-target model), Right DBP48+D (alternate target model). D, DBP57 bound strongly to its initial design target as well as an alternate target that contained an identical 6 bp stretch (ATCCAG) at the binding interface. Left: DBP57+E (on-target model), Right DBP57+C (alternate target model).

FIG. 5. Full competition assays for all DBPs designs. A-J, Relative binding activity (Median PE/FITC normalized to the no-competition sample) from flow cytometry analysis in yeast display competition assays for designs DBP1, DBP3, DBP5, DBP6, DBP9, DBP23, DBP35, DBP48, DBP56, and DBP62, respectively, with all possible DNA base mutations at each position of the competitor oligo. Heat maps show the mean of both replicates. Competitor mutations where competition was weak suggest incompatibility with binding to the competitor oligo. Arrows indicate base positions contacted with hydrogen bonds or hydrophobic contacts to base atoms in the design model. DBP48 was analyzed with sequence D due to its improved binding signal and nearly identical modeled binding sites. All other designs were analyzed with their designed target sequence. In several cases we observed extra specificity beyond the positions directly involved in hydrogen bonding and hydrophobic contacts. For example, DBPs 6 and 9 exhibit specificity for a 6 nucleotide stretch (TGCACA) with peripheral dependence on T8 and A13. This specificity is most likely explained by effects of shape readout that are not considered by Rosetta™ modeling of the designs. DBP62 appears dependent on bases peripheral to the binding site (e.g. C11).

FIG. 6. Purified designs bind their respective dsDNA targets in vitro by biolayer interferometry. Binding of purified miniprotein designs to the DNA target with BLI. Each line represents biotinylated dsDNA target dilutions by ⅓. K_{D} values are indicated above each plot. DBP48 was analyzed with the sequence D dsDNA target.

FIG. 7. Comparison of designed DBPs with nearest native structures by target motif or protein structure. A-C, Alignment of DBP designs to PDB structures containing an identical DNA binding site motif. DBP designs found substantially unique solutions for binding the same DNA sequence found in the native structures. Native structures are shown aligned to the DBP design. DNA sequence matches were found by creating a set of all contiguous DNA binding site motifs in the PDB where any atom of a protein residue was within 5 Å of an atom in the contiguous DNA sequence motif. DBP35 was designed against the DNA sequence from the crystal structure of the 1L3L PDB. D-H, Structural alignment of DBP designs to nearest PDB structures by TM-align. TM-align searches were performed on protein-DNA co-complex structures in the PDB to identify the nearest native protein scaffold. Nearest structures are shown aligned to the DBP design. Consistent with the use with native metagenome structures, most DBP designs had close matches to scaffolds in the PDB but altered docking configurations relative to DNA. The fine sampling of docking modes allows the method to identify binders to substantially unique DNA sequences. I, Computed statistics on native DBPs in the PDB with a TM-score to each design above 0.65 redesigned in the presence of the designed DBP's DNA target motif. Original DNA motifs in the native structures were replaced with the recognized motifs from each designed DBP. In cases where the register of the DNA in the crystal structure complex did not match the design model, we systematically slid the design motif sequence, exploring all possible offsets and generating rethreaded structures for each sequence alignment. Next, we used LigandMPNN to redesign the entire sequence of each native complex followed by sidechain relaxation using Rosetta™ FastRelax. To assess the resemblance between redesigned natives and designed DBP motifs, we examined whether the same amino acids formed hydrogen bonds with the same DNA base atoms (motif interaction recovery). The native redesign method was able to achieve full motif interaction recovery for DBPs 6 and 35, but not the remainder of analyzed designs. J, Analysis of sidechain preorganization for recovered motifs residues by average top two RotamerBoltzmann score. Violin plots show the distribution of avg_top_two_rboltz among recovered interacting residues for each design. Individual data points are shown for designs with full motif atom recovery.

FIG. 8. Use of DBPs to direct transcriptional repression in E. coli. A, Vectors encoding the repressor variants were constructed with a repressor under control of the IPTG-inducible P_{Tac} promoter. A synthetic promoter containing the designed DBP binding sites (blue text) around the −10 and −35 elements (red text) was used to control expression of YFP. Alternative synthetic promoters were used for the flexibly linked DBPs (tethered) to optimize the orientation and spacing of the binding sites (from top to bottom SEQ ID NOs: 64-69). B, Fold repression was not observed at 1 mM IPTG induction as determined by flow cytometry analysis of cells containing single DBP domains (DBP57, DBP48) and tandem linked DBP domains used as repressors. n=4. C, Repression screen of 96 DBP-TetR designs revealed substantial repression for at least two variants incorporating DBP57 and DBP48. Fold repression was determined for cells induced at 0.1 mM IPTG. n=1. D, Design models of the indicated DBP-TetR variants incorporating DBP57 (top) and DBP48 (bottom). E, Representative histograms of YFP fluorescence from E. coli cells transformed with DBP-TetR NOT circuits. Fold repression of YFP was ˜8.1× and ˜3.4× for DBP57-TetR (upper left) and DBP48-TetR (lower right) repressor variants, respectively, when encoded with their cognate promoters upon induction with 1 mM IPTG. Fold repression (mean of 8 biological replicates) is indicated in each subplot showing flow cytometry histograms of measured YFP fluorescence for empty cells, uninduced cells with repressor vector, and induced cells with repressor vector (1 mM IPTG).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, “about” means +/−5% of the recited value.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal amino acids are optional, and may be deleted.

In various embodiments, 1, 2, 3, 4, or 5 amino acids may be deleted from the N-terminus and/or the C-terminus of any of the polypeptides disclosed herein.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical, not including any amino acid insertions at identified insertion sites (i.e., any insertions are not considered when determining percent identity to the reference polypeptide), to the amino acid sequence selected from the group consisting of SEQ ID NO:1-52, wherein the polypeptide is a sequence-specific DNA-binding polypeptide.

The inventors disclose a computational method for the design of small DNA binding proteins (DBPs) that recognize specific target sequences through interactions with bases in the major groove. The method was employed in conjunction with experimental screening to generate binders for six distinct DNA targets. These binders exhibit specificity closely matching the computational models for the target DNA sequences at as many as six base positions and affinities as low as 20-100 nM. The designed DBPs function in both Escherichia coli and mammalian cells to regulate repression and activation of transcription of neighboring genes. Thus the polypeptides can be used, for example, to modulate transcription in living cells; to edit specific DNA bases in a genome by fusion with a base editing domain; to nick or cleave DNA at specific sites by fusion with a nickase or nuclease domain; or to integrate DNA at specific sites in a genome by fusion with a recombinase or integrase domain.

The amino acid sequences of SEQ ID NO:1-52 are shown in Table 1, together with the DNA target sequence that each polypeptide binds to. Table 1 also includes a column listing a letter and SEQ TD NO designating the DNA target bound; the nucleic acid sequence corresponding to the letter and SEQ TD NO designating the DNA target is listed in Table 2.

TABLE 1
Design amino acid sequences

| SEQ ID | Design | Design Sequence (recognition helix) | |
|---|---|---|---|
| NO | Name | ...KO Mutations (BOLD) | DNA Target Bound |
| 1 | DBP001 | TARELEVAALIAQGRSNREIAEELNISERT | A (SEQ ID NO: 53) |
| | | VERYVRRILRKLGLRNRAQIAAWVIRRS | |
| 2 | DBP003 | TKREREVLKLIAEDYGNKEIANRLNISERT | A (SEQ ID NO: 53) |
| | | VERYIRRILRKLGLKNRAELVRYAIRHG | |
| 3 | DBP005 | GFGKAVKAKRAELGLTQAEFAERAGLSRRT | A (SEQ ID NO: 53) |
| | | IIRIEQGKVKATSTTAEKIAAALGTTVQEL | |
| | | EQA | |
| 4 | DBP006 | DWAARAAAARRLRKERGLTQAELGELAGVS | B (SEQ ID NO: 54) |
| | | RTTVSRIELGRPDVSQASVDAVLAVL | |
| 5 | DBP007 | PLAELGKAIREARKKKGLTQEEVAKAAGVS | B (SEQ ID NO: 54) |
| | | RATVQRLELGKAKSIAPEKLAAIAKVVGL | |
| 6 | DBP009 | DWERRCAYARRARKELGLTQAELGELAGVS | B (SEQ ID NO: 54) |
| | | RTTVSRIERGKPDVSEASVEAVLAVL | |
| 7 | DBP010 | PLAEIGRAIKEARKRRGLTQAEVAEAAGVS | B (SEQ ID NO: 54) |
| | | RATVQRLELGKAKSIAPEKLAAIARVVGL | |
| 8 | DBP011 | VGEWVKRKRKEKGLTQEELAKLLGTSRATV | B (SEQ ID NO: 54) |
| | | QRIELGKKAPTPEQLERARRILEE | |
| 9 | DBP023 | PLAELGKAIKEARKRKGLTQAEVAKAAGVS | B (SEQ ID NO: 54) |
| | | RATVQRLELGKAKSIRPDKLRAILEVVGL | |
| 10 | DBP024 | PLAELGRAIREARRRRGLTQEEVARAAGVS | B (SEQ ID NO: 54) |
| | | RATVQRLELGKAKRIRPEKLAAIARVVGL | |
| 11 | DBP025 | PLAEIGKAIKEARKEKGLTQEEVAKAAGVS | B (SEQ ID NO: 54) |
| | | RATVQRLELGKAKSMRPEKLAAIAKVVGL | |
| 12 | DBP026 | DPILELLLEGEHTATELMRRLGLSYRTVRS | B (SEQ ID NO: 54) |
| | | RLRSLVRQGIIGYRHTGRVVYYVRDPERVR | |
| | | ELMAR | |
| 13 | DBP028 | SPLVLAILEGVARGRTPAEIAKELGVSRRT | A (SEQ ID NO: 53) |
| | | VQNILQYLRRKHKLSLEELVPFARRVLAAR | |
| 14 | DBP031 | LAAEIKRLRREAGLTQRELAERMGVSRYTV | B (SEQ ID NO: 54) |
| | | QRYELGKRTPSPEELERILAALGV | |
| 15 | DBP035 | GFGRAVKEKRKELGLTQKEFAEKAGLSRRT | B (SEQ ID NO: 54) |
| | | IIRIERGYIVPPKATKEKIAKALGTSVEEL | |
| | | EQA | |
| 16 | DBP040 | SAERHFLAVAEAGSYRRAAEILGVRRDTVR | A (SEQ ID NO: 53) |
| | | RAVLRIERKLGAPLFRREPV- | |
| | | LTLTPLGRELYERLQ | |
| 17 | DBP043 | MEELKKLLESGDPKLQGEAVKKIREKLGLT | G (SEQ ID NO: 58) |
| | | QREFGKKIGVGQAKVSRIEAGKIKLTPELK | |
| | | EKILE | |
| 18 | DBP044 | KEERVLAALEAHPWSTLAEIAELTGLSRST | G (SEQ ID NO: 58) |
| | | VSRILSRLRKEGKCDSRREGRKVRYWLVRR | |
| 19 | DBP045 | DREIEEFRREVRERMAEQGLTQADLARRSG | H (SEQ ID NO: 59) |
| | | LSRNTISRFLRGKTRPTPATVEAIRRALGL | |
| | | PA | |
| 20 | DBP046 | RRELSPLARARKAAGLTQRELAEKAGVGTA | H (SEQ ID NO: 59) |
| | | TISRIERGRRPFSRLPPEKQERIAEILGVS | |
| | | VAELE | |
| 21 | DBP047 | MTPEERERAKEIGREIRELRRERGLTQREL | D (SEQ ID NO: 70) |
| | | ADLLGVSRSTVSDIESGRRLPSEELLRRIR | |
| | | EILGV | |
| 22 | DBP048 | MTPEEIAEAKRIGKEVKERRKELGLTQREL | D (SEQ ID NO: 70) |
| | | AEKLGVSRSTVSDIENGRRLPSEELLKKIK | |
| | | EILGV | |
| 23 | DBP049 | MEELEERILALLREEWPRGLGAAEIARRLG | J (SEQ ID NO: 71) |
| | | VPRSKVRTALRRLVAEGRVRVVRGRYSRYV | |
| | | AVEP | |
| 24 | DBP050 | GNPRKEKILEALCRGPRTSTEIAREIGVST | J (SEQ ID NO: 71) |
| | | RTAAGLLQGLVRQGLARPRRRGRRVYYELA | |
| | | DPSIC | |
| 25 | DBP051 | MEADPAVVFGRRLRAARRAKGLTQAELAER | E (SEQ ID NO: 56) |
| | | AGLGQGTISRYEKGRTLPSPEQVEKLLAAL | |
| 26 | DBP052 | RPLTPAEVFGRELRRLRRAAGLTQAELAER | E (SEQ ID NO: 56) |
| | | AGIGQGTVSRYEHGRRLPSPEEQERLLAAL | |
| −27 | DBP053 | RKLSPYERFGREIKERRKEAGLTQAELAEL | E (SEQ ID NO: 56) |
| | | AGVGQATVSRIEKGEKVSPEILEKIREALE | |
| | | KA | |
| 28 | DBP054 | RVKTPFERFGEFVKRERAKAGLTQAELAKL | E (SEQ ID NO: 56) |
| | | AGVGQSTVSRIEKGKKCSPELREKIVKALK | |
| | | AV | |
| 29 | DBP055 | RKKSPLEIIGERIKKERKELGLTQAELAKL | E (SEQ ID NO: 56) |
| | | AGIGQSTVSRIEKGEKCSQRIIEKIFKALA | |
| | | AV | |
| 30 | DBP056 | PPPTPFEVAGARIKEERAKLGLTQAELAKV | E (SEQ ID NO: 56) |
| | | AGVGQATVSRIEKGRKCSWELIEKIFEALK | |
| | | KV | |
| 31 | DBP057 | MVLTPMERIGEFIKRARREAGLTQRELAEL | E (SEQ ID NO: 56) |
| | | AGVGQSTVSRIEKGEKCSPELVEKILEALR | |
| | | KV | |
| 32 | DBP058 | AWTGEQLREFRKKLGLSQREFGELLGVGQS | E (SEQ ID NO: 56) |
| | | TVSRVEHGGELGPATRARLQARVDELVAEY | |
| | | KASQ | |
| 33 | DBP059 | MKELGKKIKERRKKLGLTQAQLSELSGVGQ | E (SEQ ID NO: 56) |
| | | GTISRLEQGRGNPSPKILEKIEKVLKELEK | |
| 34 | DBP060 | DIEKIAKAVKELREELGLTQAEFAKKIGIG | E (SEQ ID NO: 56) |
| | | QGTLSRFEKGGVLSPKTMERLLKALEKEFG | |
| | | FDVKK | |
| 35 | DBP061 | GAKEKLWEFLLELAKKGLPFKLPSAEEIAR | F (SEQ ID NO: 57) |
| | | RLGVRRRTVIGQLQSFVREGRIKLKRGVVY | |
| | | SVNE | |
| 36 | DBP062 | KEELEKLLKIIESLPKKFREVIILKFVEGL | F (SEQ ID NO: 57) |
| | | SYTEIAERLGVSRGAVYSRLRSALKKIEEA | |
| | | LKK | |
| 37 | DBP068 | PLSGKELGELIKKYRDEKGLTQAEFAKLAG | C (SEQ ID NO: 55) |
| | | LGQGTISRLEKGVDRNGKEYHPGEEIREKV | |
| | | LKAIA | |
| 38 | DBP069 | MKEEGRKLKELRERLGLTQAELAEALGLGQ | C (SEQ ID NO: 55) |
| | | STISRLERGRKEISPEVWEKALALLE | |
| 39 | DBP071 | NTELLKQKIKEKGLSREEVAKKLGISRNTL | H (SEQ ID NO: 59) |
| | | TQKILGHRKFSPEQIEILKELLGLSEEEVK | |
| | | EIFFP | |
| 40 | DBP080 | ATAAQRWRLSPRETEVLELLINGYTNKEIA | H (SEQ ID NO: 59) |
| | | SALNVSVRTVEVHIRRVLRKANVRRRVELV | |
| | | AKYYG | |
| 41 | DBP081 | TPREREVLNLLAQGYSNREIAERLNISEKT | A (SEQ ID NO: 53) |
| | | VKNYVRNILRKLGVRNRVEAVRWWLAVR | |
| 42 | DBP082 | NRIDSLSPREREVLRLIAQGYNNKEIAEQL | A (SEQ ID NO: 53) |
| | | NISEKTVKVHVRRILRKLNVHNRAELVNLK | |
| 43 | DBP083 | PREREILRLLAEGKNAWEIAQILNISVRTV | A (SEQ ID NO: 53) |
| | | RNHLRNAMRKLGARNRVQAVARALRLG | |
| 44 | DBP085 | TLSQLTPQEMRIARLASEGMPNREIATRLF | A (SEQ ID NO: 53) |
| | | ISPRTVEWHLRRAMRKLGVRNRTQMARRID | |
| | | TRL | |
| 45 | DBP086 | TKREAEVLELLSRGRSNKEIASILHISVRT | A (SEQ ID NO: 53) |
| | | VEWYIRRILRKLGVKNRVEAVRTAKAQG | |
| 46 | DBP087 | GVERLTPREKRVAHLAAQGLTNREIAEALH | A (SEQ ID NO: 53) |
| | | ISPRAVENHLRRILRKLGIRRRRELPEALG | |
| | | E | |
| 47 | DBP088 | PREMEVLNLMAQGYNNKEIAARLGISEKTV | A (SEQ ID NO: 53) |
| | | KNHVRRILRKLGVRNRVQAVIIAQRNG | |
| 48 | DBP089 | SPAAFDKLTARELAVARLVAQGLPNREIAA | A (SEQ ID NO: 53) |
| | | ALHISPRAVEAHLRKIYRKLGIRRRRELAA | |
| | | LLA | |
| 49 | DBP090 | NKYQLSLLESAFQSNRYPDISQRATLASQT | A (SEQ ID NO: 53) |
| | ( | GLPERRIKIWFQNRRQRWKRKK | |
| 50 | DBP035 | GFGRAVKEKRKELGLTQVEFAEKAGLSRRT | B (SEQ ID NO: 54) |
| | variant | IINIERGYIVPQKATKEKIAKALGTSVEEL | |
| | | EQA (SEQ ID NO: 51) | |
| 51 | DBP035 | GFGRAVKEKRKELGLTQVEFAEKAGLSRRT | B (SEQ ID NO: 54) |
| | variant | IINIERGYIVPMKATKEKIAKALGTSVEEL | |
| | | EQA | |
| 52 | DBP035 | GFGRAVKEKRKELGLTQVEFAEKAGLSRRT | B (SEQ ID NO: 54) |
| | variant | IIKIERGYIVPQKATKEKIAKALGTSVEEL | |
| | | EQA | |

TABLE 2
DNA target sequences

| SEQ ID NO | Name | Sequence |
|---|---|---|
| 53 | A | TAGCAGGATGTGT |
| 54 | B | GCAGATCTGCACATC |
| 55 | C | CGGCTGGATTACCG |
| 70 | D | CGACACCTGACGCG |
| 56 | E | CGCTATCCAGAGCG |
| 57 | F | CGCGATGCTTCTCG |
| 58 | G | CGAGAACATAGTCG |
| 59 | H | CGGGGAAACGCCCG |
| 71 | J | CGGAGGTAATGACG |

In other embodiments, the polypeptide comprises an amino acid sequence 50%, 55%, 60%, 65%, 70%, 75%, 80%, 85%, 90%, 91%, 92%, 93%, 94%, 95%, 96%, 97%, 98%, 99%, or 100% identical, not including any amino acid insertions at identified insertion sites (i.e., any insertions are not considered when determining percent identity to the reference polypeptide), to the amino acid sequence selected from the group consisting of SEQ ID NO:1, 2, 4, 15, 21-23, 25, 26, 30, 31, 34, 36, 38, 44, and 49-52.

In some embodiments, the polypeptides comprise an amino acid sequence at least 75% identical to the amino acid sequence selected from the group consisting of SEQ ID NO: 1-52, or SEQ ID NO:1, 2, 4, 15, 21-23, 25, 26, 30, 31, 34, 36, 38, 44, and 49-52. In other embodiments, the polypeptides comprise an amino acid sequence at least 90% identical to the amino acid sequence selected from the group consisting of SEQ ID NO:1-52, or SEQ ID NO:1, 2, 4, 15, 21-23, 25, 26, 30, 31, 34, 36, 38, 44, and 49-52. In further embodiments, the polypeptides comprise an amino acid sequence at least 95% identical to the amino acid sequence selected from the group consisting of SEQ ID NO: 1-52, or SEQ ID NO:1, 2, 4, 15, 21-23, 25, 26, 30, 31, 34, 36, 38, 44, and 49-52. In still further embodiments, the polypeptides comprise the amino acid sequence selected from the group consisting of SEQ ID NO:1-52, or SEQ ID NO:1, 2, 4, 15, 21-23, 25, 26, 30, 31, 34, 36, 38, 44, and 49-52. In all of these embodiments, the percent identity requirement does not include any amino acid insertions at identified insertion sites (i.e., any insertions are not considered when determining percent identity to the reference polypeptide),

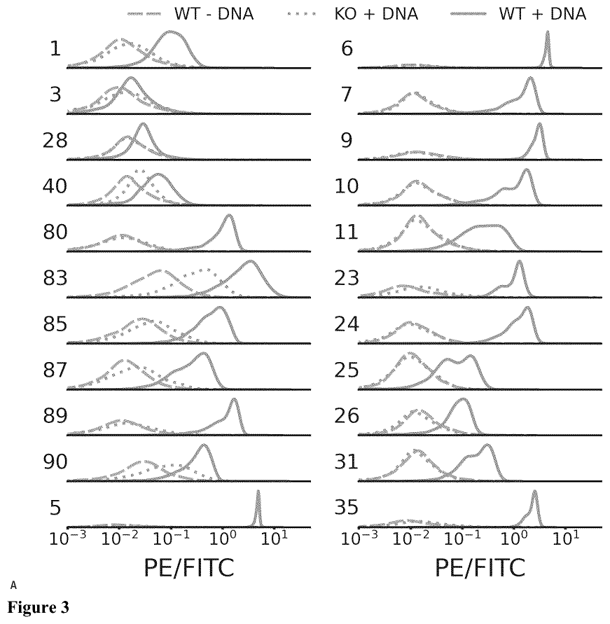

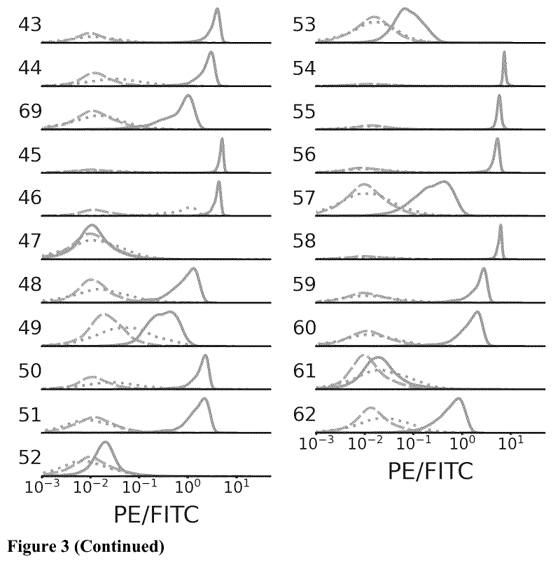

Table 1 shows some residues in bold font that were identified as playing a role a key role in the interface between the polypeptides and their DNA targets, as demonstrated in FIG. 3. Thus, in some embodiments, residues in bold font are conserved (i.e., identical) relative to the reference sequence. For example, R29, E32, and R33 are in bold font in SEQ ID NO:1 (DBP001). In one embodiment, the polypeptide would have residues R29, E32, and R33 relative to SEQ ID NO:1. Those of skill in the art can identify the relevant residues from Table 1 for the other sequences.

Table 1 also presents some residues as underlined; these are all of the residues at the binding interface with the target DNA. In some embodiments, underlined residues are conserved relative to the reference sequence. For example, residues 28-42 are underlined in SEQ ID NO:1 (DBP001). In one embodiment, residues 28-42 would be conserved, relative to SEQ ID NO:1, in the polypeptides of the disclosure. Those of skill in the art can identify the relevant residues from Table 1 for the other sequences.

As disclosed in the examples that follow, the contributions of each amino acid to binding for additional designs, high-resolution footprints of the binding surface as generated was assessed by sorting site saturation mutagenesis libraries (SSMs) in which every residue was substituted with each of the 20 amino acids one at a time for DBPs 1, 6, and 35. Permissible substitutions for many of the polypeptides of the disclosure are provided in columns 2 and 3 Tables 4-19. Table 3 shows the correspondence between the Table number and the sequences to which the Table relates. For example, Table 4 shows SSM and other information relative to SEQ ID NO:21.

TABLE 3

| Table number | Reference sequences |
|---|---|
| Table 4 | SEQ ID NO: 21 |
| Table 5 | SEQ ID NO: 22 |
| Table 6 | SEQ ID NO: 23 |
| Table 7 | SEQ ID NO: 25 |
| Table 8 | SEQ ID NO: 26 |
| Table 9 | SEQ ID NO: 30 |
| Table 10 | SEQ ID NO: 31 |
| Table 11 | SEQ ID NO: 34 |
| Table 12 | SEQ ID NO: 36 |
| Table 13 | SEQ ID NO: 38 |
| Table 14 | SEQ ID NO: 44 |
| Table 15 | SEQ ID NO: 49 |
| Table 16 | SEQ ID NO: 1 |
| Table 17 | SEQ ID NO: 2 |
| Table 18 | SEQ ID NO: 4 |
| Table 19 | SEQ ID NO: 15 and 50-52 |

Thus, in some embodiments, substitutions relative to the reference sequence are selected from residues listed in columns 2 or 3 of one of Tables 4-19. In other embodiments, substitutions relative to the reference sequence are selected from residues listed in column 2 of one of Tables 4-19. Column 2 lists the “best” substitutions, which retain or improve DNA target binding activity relative to the reference sequence. Column 3 lists “permissible” substitutions, which retain binding to the DNA target sequence.

Tables 4-19 list interface residues, present at the interface between certain of the designed polypeptides and their DNA binding target. In one embodiment, only conservative substitutions, or no substitutions are permitted relative to the reference sequence at interface residues identified in Table 4-19.

Tables 4-19 list core residues, present in the core of certain designed polypeptide. In one embodiment, only conservative substitutions, or no substitutions are permitted relative to the reference sequence at core residues identified in Table 4-19.

Tables 4-19 list insertion sites, where an insertion may be made in certain reference polypeptides. For example. Table 4 shows that insertions can be made at residues 24-26, 35-37, and 46-52 relative to SEQ ID NO:21 These residues are referred to as “insertion sites”. Insertions are permissible at these regions because they are (1) flexible loop regions that can accommodate residue insertions without altering the backbone conformation of the polypeptide, and (2) they are not involved in interactions with the DNA target sequence. Amino acid insertions may be single residues, multiple residues, or functional domains. The insertion may be any one or more amino acid, and may comprise a functional domain as described below, or one or more amino acids for additional spacing or for any other purpose. In one embodiment, an insertion in the loop regions is 1-3, 1-2, 1, 2, or 3 amino acids in length. In other embodiments, one or more other DNA binding domains may be inserted at an insertion site. For example, if one wanted to create a binder with longer sequence recognition, a second polypeptide of the disclosure could be fused at an insertion site, giving the fusion more flexibility. Similarly, one or more transcriptional activation domains, nuclease domains, base editing domains, nicks domains, and/or integrase/recombinase domains may be inserted at an insertion site.

The polypeptides of the disclosure may include any such insertion, and the polypeptide would still comprise the reference amino acid sequence, with an interruption at the site of insertion.

In one embodiment, an amino acid insertion is present, but does not result in elimination of any residues in the polypeptide. In another embodiment, an amino acid insertion is present, and does result in elimination of 1, 2, 3, or 4 contiguous insertion sites. In another embodiment, no insertions are present.

The position of residues in the polypeptides of the disclosure are “relative to” the position of residues in the reference sequence; this does not necessarily mean that the residue number in the polypeptide of the disclosure will be identical to the residue number in the reference sequence. Those of skill in the art will understand that the polypeptides of the disclosure may be fused to other functional domains, including N-terminal domains, or may include insertions at residues relative to the reference sequence, as noted above and in Tables 4-19.

TABLE 4
DBP047 MTPEERERAKEIGREIRELRRERGLTQRELADLLGVSRSTVSDIESGRRLPSEELLRRIREILGV
(SEQ ID NO: 21)

| pos- | | | at_ | protein_ | loop/ |
|---|---|---|---|---|---|
| ition | best | tolerable | interface | core | insertion |
| 1 | S, M, I, L, Y | A, E, W, N, T, V | | | |
| 2 | K, T, G, V | W, F, D, I, N | | | |
| 3 | C, P, N, L | S, G, W, M | | | |
| 4 | Q, Y, M | A, N, W, G, H, E, K, R | | | |
| 5 | C, R, E, A, N | P, K, I, M, F | | | |
| 6 | G, R, I | F, A, C, K, S, T | | | |
| 7 | D, E, R | W, P, I | | | |
| 8 | L, R | K, H, I | | | |
| 9 | R, A, E | K, P, C, F | | | |
| 10 | K | L, G, Q, N, P, I, M, R | | | |
| 11 | C, M, E, A, L, T | P, K, Y, N, H, Q, W | | | |
| 12 | C, I, Y | P, Q, V, F, A, D, S, L | | | |
| 13 | G | V, F, T, A, C, Y, M | | | |
| 14 | R | T, E, V, I, C, W, Y, N, K, | | | |
| | | L, A, F | | | |
| 15 | A, E, M | S, H, K | | | |
| 16 | L, T | E, I, M, D, V | | X | |
| 17 | R, S | Q, W, G | | | |
| 18 | C, E, V, K | P, I, Q, D | | | |
| 19 | F | Y, R, L, Q, M, N, K | | | |
| 20 | R | W, M | | | |
| 21 | R | S, M, V | | | |
| 22 | M, T, A | P, H, E, I, D | | | |
| 23 | W, R | Y, G, P, S, D, M, F, V, K, | | | |
| | | H, T | | | |
| 24 | Y, G, C | T, F, W, M, D, R | | | X |
| 25 | F, L, S, R, W | K, P, T, V, Y, C | | | X |
| 26 | Y, F, V, L | T, N, G, M, W, I, R | | | X |
| 27 | Q | K, H, L, A, I, F | X | | |
| 28 | R | M, S, C, K | | | |
| 29 | K, S, N, E, H | R, C, A, G, V, T, L, P, D | | | |
| 30 | H, I, E, Y | V, L, M, F, Q, T, C | | | |
| 31 | A | D, K, T, V, S | | | |
| 32 | G, K, R, L, V, F, | M, S, A, P, N, H, Q, T, I, | | | |
| | D, W | Y, E | | | |
| 33 | P, G, L, C, T | I, F, H, A, Q, D, N | | | |
| 34 | A, L, R, N | C, T, K, M, P | | | |
| 35 | G, Y, R, A | W, V, H, C | | | X |
| 36 | R, Y, V | K, L, I, C, G, A, N | X | | X |
| 37 | S | D, E, H, T, V | X | | X |
| 38 | R | M, Q | X | | |
| 39 | S, L | T, W, Y | X | | |
| 40 | Y, F | H, G, T, C, Q | X | | |
| 41 | V, I | A, D | | X | |
| 42 | S | | X | | |
| 43 | G | M, D, N, Y, A, E, V, C | X | | |
| 44 | R, A | K, Y, G, Q, T, I, L, W, D, | | | |
| | | F, S | | | |
| 45 | E | W, Y, D, T, H, G, I, S | X | | |
| 46 | T, S | R, E, G, N, C | X | | X |
| 47 | R, G | L, P, M, Y, V, I, N, H | | | X |
| 48 | R | S, W | X | | X |
| 49 | R, Q | W, G, I, S, C, Y | X | | X |
| 50 | R, L, S | K, N, P, D, E | X | | X |
| 51 | K, P, S, G, A | R, F, N, Y, I | | | X |
| 52 | R, K, S, M | P, G, H, V, Y, N | X | | X |
| 53 | C, E, L, I | W, G, D, K | | | |
| 54 | R, E, V, Q | C, K, M, A, N, G | | | |
| 55 | R, W | K, A, V, L, C, Y, T, Q, F, | | | |
| | | G, S | | | |
| 56 | C, E, L | Q, T, R, F | | | |
| 57 | C, R, H, K | M, Q, Y, P, E, V, D, W | | | |
| 58 | R, S | Q, I, L | | | |
| 59 | Y, I, E, M | K, T, N | | | |
| 60 | N, R, P | G, K, L, C, H, T, F, V | | | |
| 61 | M, E, H | F, Y, R, G | | | |
| 62 | W, C, I, N, Q | D, E, R | | | |
| 63 | C, L, N | Q, R, F | | | |
| 64 | S, G | K, E | | | |
| 65 | V, D | T, K, R, M, E | | | |

TABLE 5
DBP048 MTPEEIAEAKRIGKEVKERRKELGLTQRELAEKLGVSRSTVSDIENGRRLPSEELLKKIKEILGV
(SEQ ID NO: 22)

| pos- | | | at_ | protein_ | loop/ |
|---|---|---|---|---|---|
| ition | best | tolerable | interface | core | insertion |
| 1 | G, K, L, Y, M, R | W, Q, A | | | |
| 2 | R, S, G, T, A, K | C, V, P, Q, W, L | | | |
| 3 | R, V, M, A, P, I, H, E, Q, S, | F, L | | | |
| | W, T, K | | | | |
| 4 | S, I, T, Y, W, M | D, G, H, V, E, K | | | |
| 5 | Y, F, P, W, N, L, Q, M, E, I, | | | | |
| | D, S, H, R, G, C, K, V | | | | |
| 6 | N, I, V, A, T, K | F, Q, L, W, R, G, | | | |
| | | H | | | |
| 7 | N, I, Y, E, H, A, V, R, P, T, | Q, G | | | |
| | D, M, S, F | | | | |
| 8 | H, L, A, K, R, D, E, S, C, Y, | P | | | |
| | W, F, G, I, Q, T, V | | | | |
| 9 | K, R, F | M, E, G, P, N, A, | | | |
| | | H, Q, W | | | |
| 10 | Q, I, R, H, K | | | | |
| 11 | H, D, V, N | A, K, C, W, L, R, | | | |
| | | I, M | | | |
| 12 | P, Q, W, R, I, G, L, D, M, | A, T | | | |
| | F, K | | | | |
| 13 | K, C, G, N, W, Q | I, R, S, P, L, H, | | | |
| | | V | | | |
| 14 | L, P, Q, G, A, R, K, V, H, E | M | | | |
| 15 | D, Q, G, E, H, I, S, V, A, | Y | | | |
| | T, C | | | | |
| 16 | G, K, V, Q, F, H, W, R | L, D, E | | X | |
| 17 | K | | | | |
| 18 | Q, D, E, L, A, N, S, P, M | W | | | |
| 19 | T, N, Y, M, R, K, A, L, C, V | G, Q, P | | | |
| 20 | R | | | | |
| 21 | R, K, H, V, P | | | | |
| 22 | Q, R, M, T, E, K, F, C, L | N, D, P | | | |
| 23 | R, F, E, L, A, G, V, Y, C, N, | | | | X |
| | W, D | | | | |
| 24 | R, D, V, I, K, G, T, Q, H, E, | P | | | X |
| | A, N, W, L, S, M, F | | | | |
| 25 | K, N, W, R, A | S, H, E, C, Y, L, | | | X |
| | | F, P, G, V | | | |
| 26 | W, F, Y, V, L | P, T, R | X | | X |
| 27 | Q | | X | | |
| 28 | R, L | | | | |
| 29 | R, T, E, N, V, I, M, S, A, | H, P, G | | | |
| | L, Q | | | | |
| 30 | E, S, L, V, I, G, Q, T, M | | | | |
| 31 | A, G, S | | | | |
| 32 | T, Y, F, E, S, I, H, L, V, C, | | | | |
| | M, G, Q, A, P, K, D | | | | |
| 33 | N, C, Y, W, K, Q, E, I | H, R, L, G, S, V | | | |
| 34 | M, A, Y, N, W, L, C, T, I, G, | | | | |
| | V, F, S | | | | |
| 35 | Q, V, T, H, K, P, G, R, N, L, | W, E | | | X |
| | I, M | | | | |
| 36 | K, Y, R | A, N, C, V, G, H, | X | | X |
| | | M | | | |
| 37 | A, G | K, S, R | X | | X |
| 38 | R | | X | | |
| 39 | S | | X | | |
| 40 | F, L, Y | T, M, A, W, G, C, | X | | |
| | | H | | | |
| 41 | I, V, C | | | X | |
| 42 | S, G | | X | | |
| 43 | C, G, M, A, V, H, S | D, T | X | | |
| 44 | N, Y, V | M, I, G, Q, L | | | |
| 45 | E | | X | | |
| 46 | R, C, V, S, I, P, L, T | N | X | | X |
| 47 | I, P, G, N | T, S | | | X |
| 48 | R, G | | X | | X |
| 49 | R | | X | | X |
| 50 | Y, S, W, V, Q, I, F, M, K, | H, L, C | X | | X |
| | R, G | | | | |
| 51 | L, P, T, C, V, Q, M, K, Y, | W, G | | | X |
| | A, S | | | | |
| 52 | R, K, M | G, W, S, V, L, A | X | | X |
| 53 | G, Y, Q, W, T, M, C, A, E, K, | | | | |
| | I, V, S, P, L, H, R, F | | | | |
| 54 | K, G, W, H, N, R, A, P, Q | D, Y, S, E, L, V | | | |
| 55 | V, F, K, R, N | T, L, Y, P, M | | | |
| 56 | K, A, I, R, C | E, G, Q, L, F, H | | | |
| 57 | I, K, M, F, L, S, R, H | V | | | |
| 58 | I, K, G, V, M, R, P | Y, L, C | | | |
| 59 | V, C, T, Q, M, I, H, F | Y, G, L | | | |
| 60 | M | I, R, S, E, K, V, | | | |
| | | A, D, H | | | |
| 61 | Q, M, R, L, E, N, V, T, G, D, | F | | | |
| | H, A, K, I, Y | | | | |
| 62 | H, T, W, Y, E, F, K | A, I, C, M, G | | | |
| 63 | I, H, M, F | R, C, T, Y, L, S, | | | |
| | | Q | | | |
| 64 | N, I, Y, C, G, T, R, A, V, K, | | | | |
| | M, Q, H, F | | | | |
| 65 | N, S, F, V, Y, H, T, L, A, | P, W, D, Q | | | |
| | I, R | | | | |

TABLE 6
DBP049 MEELEERILALLREEWPRGLGAAEIARRLGVPRSKVRTALRRLVAEGRVRVVRGRYSRYVAVEP
(SEQ ID NO: 23)

| pos- | | | at_ | protein_ | loop/ |
|---|---|---|---|---|---|
| ition | best | tolerable | interface | core | insertion |
| 1 | I, Q, P, N, W, A, M, D, V, H | | | | |
| 2 | K, I, Q | S, H, R, P, G, L, E | | | |
| 3 | F, G, E, W, T, M | | | | |
| 4 | D, W, E, F | P, M, G, T, K, L | | | |
| 5 | W, F, M, A, V, N, K, E, Q | | | | |
| 6 | P, R, G, D, F, T | Y, A, W, E | | | |
| 7 | S | N, D, G, H, A, R | | | |
| 8 | E, Y, P, G, C, S, H, K | N, T, Q, M, F, A, | | X | |
| | | D, I | | | |
| 9 | A, N, D, C, E, G, R | M, H, V, T, Q, I, | | | |
| | | S, W, L | | | |
| 10 | F, L, I, P, N, Y | V, R, T, K, M, A | | | |
| 11 | V, F, W, P, H, A, R, K, Y, | L | | | |
| | Q, G, S, D | | | | |
| 12 | Q, G, D | H, N, V, A, E, M, L | | X | |
| 13 | E, V, A, W, S, G, C, L, D | I, H, N, R | | | |
| 14 | R, Y, H, G | W, K, Q, E | | | |
| 15 | Q, W, H, F, G, D, Y, R, S, | | | | |
| | E, L, P, A, V, I | | | | |
| 16 | I, W, L, V, F, S, E | | | | X |
| 17 | P, R, G, Y, N, H, K, C, T, | | | | X |
| | Q, D, S, F, A, L, M | | | | |
| 18 | R, K, F | | | | X |
| 19 | A, W, G, I, H, T | | | X | X |
| 20 | P, A | R, C, L | | | |
| 21 | Y, G, W, L, Q | F | | | |
| 22 | V, Y, D, G, P | H, A | X | | |
| 23 | W, Y | P, V, E, S, A | X | | |
| 24 | S, M, I, F, P | L, V, W, R, D, E, | | | |
| | | N | | | |
| 25 | Q, D, H, L, Y | C, K, E, P, G, T, | | X | |
| | | M, N, I | | | |
| 26 | S, T | W, A | | | |
| 27 | R, K, A | | | | |
| 28 | R, W, V, L, Q, Y, A, S, M, P | | | | |
| 29 | R, C, K, N, H, Q, A, D, P, | V, Y, L | | | |
| | E, G, W | | | | |
| 30 | Q, R, M, Y, G, F, L, D, E | H | | | X |
| 31 | W, C, S, F, E, V, D, P, I, Y | | | | X |
| 32 | Y, D | F, W, M, A, P | X | | X |
| 33 | Q, P | G, R | X | | |
| 34 | F, V, W, L, M, I, Q, T, N, | Y | X | | |
| | S, D, A, P, K | | | | |
| 35 | E, N | D, L, Q, I, T, Y, | X | | |
| | | H, M, P, A, F, V, | | | |
| | | G, K | | | |
| 36 | S | L, N, M, G, A, H, | | X | |
| | | D, K, E, V | | | |
| 37 | Y, W, F, Q, A, M, P, H, I, G | R | X | | |
| 38 | W, F, P, V, L, E, H, Q, G, D | Y, S, T | X | | |
| 39 | G, E, D, W, F, K, Y, V, I, T | H, A | | | |
| 40 | Q, N, D | C, E, V, G, M, H, | | | |
| | | T, S, F, L | | | |
| 41 | W, P, C | N, H, R | | | |
| 42 | T, L, V, F, I, S, Y, D, A | E, G, P, Q, C, M, | | | |
| | | R | | | |
| 43 | P, K, E, D, I, T, R, S, Q, N | W, G, A, H, L | | | |
| 44 | S, W, P, F, D, T, N, M, E | Y, G, V | | | |
| 45 | I, C, F, P, T, A, L, G, H, | | | | |
| | V, M, Y, Q, K, E | | | | |
| 46 | T, D, P, N | M, R, G, V, E | | | |
| 47 | S, P | V, F, K, Q, L, R, | | | X |
| | | N, G | | | |
| 48 | V, S, P, K, N, T, G, H, A, | | | | X |
| | R, E, I, L, C | | | | |
| 49 | S, G, F | T, V | | | |
| 50 | W, K, Y, C, R, Q, L, M, H, | | | | |
| | S, N, T, D, A, E, V | | | | |
| 51 | Q, T, F, H, Y, S, M | P, A, V | | | |
| 52 | W, P, Y, T, Q, S, C | L, M, I, F, R, V | | | |
| 53 | N, W, Y, A, C, H, G, K, F, | R | | | |
| | P, T | | | | |
| 54 | W, F, R, M, Q, L, H | G | | | X |
| 55 | W | Y, C, R | X | | X |
| 56 | F, V | Y | X | | X |
| 57 | L, Q, G | I, W, S | X | | X |
| 58 | K, R | | X | | |
| 59 | W, Y, F, H | | | | |
| 60 | R, Q, L, I, V, K, A, G, M, | | | | |
| | T, H, Y, E, C, N | | | | |
| 61 | C, N, A, G, I, R, T, P, Y, | | | | |
| | K, Q | | | | |
| 62 | N, D, Y, C, Q, H, W, K, V, | | | | |
| | G, E, P | | | | |
| 63 | R, N, S, G, D | P, A, I, E | | | |
| 64 | F, L, C, A, N, H, Q, D, R, | | | | |
| | G, P, W, Y, V, K | | | | |

TABLE 7
DBP051 MEADPAVVFGRRLRAARRAKGLTQAELAERAGLGOGTISRYEKGRTLPSPEQVEKLLAAL
(SEQ ID NO: 25)

| pos- | | | at_ | protein_ | loop/ |
|---|---|---|---|---|---|
| ition | best | tolerable | interface | core | insertion |
| 1 | G, A, F, I, N, M | Y, T, R, K, H, P, | | | |
| | | V, L, Q, W | | | |
| 2 | T, A, S, V, P, R, I, D, L, M, | Q, H, W, Y, K, N, | | | |
| | E | C, G, F | | | |
| 3 | R, G | V, L, W, N, S, Q, | | | |
| | | M, E, P, | | | |
| | | I, T, Y, F, A, H, K | | | |
| 4 | G, Y, K, P, W, S, M, F, T, D | N, H, L, A, R, V, | | | |
| | | Q, C, I, E | | | |
| 5 | C, K, T, L, V, P, D, Q | R, N, S, G, H, A, | | | |
| | | I, Y, W | | | |
| 6 | Q, K, F, R, T, E, A | S, N, P, H, G, C, | | | |
| | | D, V, M, L, I, W | | | |
| 7 | K, G, R, L | P, N, H, S, M, Y, | | | |
| | | I, Q, A, T, F, | | | |
| | | W, V | | | |
| 8 | N, T, Y, K, A, P, D, V | S, G, Q, R, L, I, | | | |
| | | H, C, E, M, F, W | | | |
| 9 | L, F, C | I | | | |
| 10 | G, R | | | | |
| 11 | K | P, R | | | |
| 12 | K, R, S, G | | | | |
| 13 | L, W | | | X | |
| 14 | R, K | | | | |
| 15 | S, K, A, H, R, I | T, M, F, Q, V, D | | | |
| 16 | K, Y, M, L, I, C, A, V | S, R, Q, W, H, T, | | | |
| | | N | | | |
| 17 | R, P | | | | |
| 18 | K, R, H, F | | | | |
| 19 | R, K, G | S, Q, L, M, T, C, | | | |
| | | A, H, F, N | | | |
| 20 | L, R, K, M | Q, C | | | |
| 21 | K, R, Q, G, S, M | H, F, C | | | X |
| 22 | Y, W, L, T, R | F, M | | | X |
| 23 | T | | X | | X |
| 24 | H, K, Q | | X | | |
| 25 | K, V, R, A | T, S, M, Q, H | X | | |
| 26 | G, T, W, R, I, K, H, F, Y, V, | S, L, C, Q, M | | | |
| | N, A, E, D | | | | |
| 27 | V, L | | | | |
| 28 | G, A | | X | | |
| 29 | I, T, A, K, F, G, Y, W, R, N, | S, M | | | |
| | C, H, V, L, Q, E, D, P | | | | |

| | | | | | |
|---|---|---|---|---|---|
| 30 | K, R, Q | | | | |
| 31 | V, A, I, D | C, T | | | |
| 32 | K, R, V | H, N, G | | | X |
| 33 | C, V, M, R, L | K, F | X | | X |
| 34 | S, R, H | G, A | X | | X |
| 35 | Q, R | | X | | |
| 36 | S, A | P, T, G | X | | |
| 37 | T | | X | | |
| 38 | I, Q | | X | | |
| 39 | S, C | | X | | |
| 40 | R, C | | X | | |
| 41 | Y | | | | |
| 42 | E, F | | X | | |
| 43 | N | T, R, K | X | | X |
| 44 | A, S, G, K | | | | X |
| 45 | R | | | | X |
| 46 | F, I, R, T, S, P | V, Y, N, H, K | X | | X |
| 47 | S, R, Q, N, I, K | F, C, T, V, W, M, | | | X |
| | | A, Y, H, L | | | |
| 48 | A, P | Q | | | X |
| 49 | H, R, K, N, V | G, A, Q, S | | | X |
| 50 | K, R, M, A, P, L | C, Q, N, H, Y, S, | | | |
| | | T, G, W, I, V, F | | | |
| 51 | F, L, K, Y | I, W, G, R, H, N, | | | |
| | | Q, T, A, | | | |
| | | S, M, C, V, P, E | | | |
| 52 | K, R, M, G, I, S, F, Y, L, N, | T, V, A | | | |
| | Q, C | | | | |
| 53 | R, K, V, T, M, Y, P | Q, I | | | |
| 54 | A, R, Q, T, Y, S, C | K, G, I, N, M, F, | | | |
| | | L, D, H, V, W, E | | | |
| 55 | K, W | | | | |
| 56 | V, D | L | | | |
| 57 | N, S, W, R, L | Q, D, Y, V, I, M, | | | |
| | | F, T, E, A, K, H, | | | |
| | | G, C | | | |
| 58 | K, T, Y, W | V, R, F, H, C, N, | | | |
| | | M, Q, G, L, A, I, | | | |
| | | S, P | | | |
| 59 | G | L, A | | X | |
| 60 | E | L, I, M | | | |

TABLE 8
DBP052 RPLTPAEVFGRELRRLRRAAGLTQAELAERAGIGQGTVSRYEHGRRLPSPEEQERLLAAL
(SEQ ID NO: 26)

| pos- | | | at_ | protein_ | loop/ |
|---|---|---|---|---|---|
| ition | best | tolerable | interface | core | insertion |
| 1 | R, E | S, A, P, G, L, W, I, Q, K, | | | |
| | | T, Y, M | | | |
| 2 | P | W, H, N, Y, T, G, S, R, M, | | | |
| | | L, K, E, Q, D | | | |
| 3 | L, A, G, P, H | N, I, M, W, K | | | |
| 4 | G, T, H, W | N, M, A, F, I, Q, K, R, P, | | | |
| | | V | | | |
| 5 | P, V, K, S, R | E, L, M, T, H, I, C, Q, N, | | | |
| | | F | | | |
| 6 | A, G, N, F | H, S, T, L | | | |
| 7 | E, H, P | Q, F, L, G, S, T | | | |
| 8 | P, L, G, K, R | V, W, E, H, I, D, S, T, N, | | | |
| | | F, A, M, Q | | | |
| 9 | F | L, Y | | | |
| 10 | G | | | | |
| 11 | R, C | M, G, K, I, A, T, V, Q | | | |
| 12 | E, K | V, A, W, Y, D | | | |
| 13 | L | F, V | | X | |
| 14 | R, F | K | | | |
| 15 | R, T | Y, S, P, Q, F, K | | | |
| 16 | L, M | S, T, N, F, H, Y | | | |
| 17 | R | Y | | | |
| 18 | R | H | | | |
| 19 | T, A | Y, L, F, Q, H, M, K, S | | | |
| 20 | A | K, E, M, Y, H, G, Q, N | | | |
| 21 | G | T, Y, W, S, H | | | X |
| 22 | K, F | D, L, A, Q, C, V, R, M | | | X |
| 23 | T | P | X | | X |
| 24 | Q | P, K, H | X | | |
| 25 | K, R | T, S, A, N, Q, M | X | | |
| 26 | R, Q, K, T, W, S, L, | I, N, Y, G, M, H, F, E | | | |
| | A, V, C | | | | |
| 27 | L, T | I, V, Y | | | |
| 28 | G | A | X | | |
| 29 | Y, K, N, R, A, Q, H, | F, L, S, M, T, I, D | | | |
| | V, G, W, E, C | | | | |
| 30 | R | F, M | | | |
| 31 | W, V, L, M, A, Q, H | F, C, I, R | | | |
| 32 | G, A, F, D | S, R | | | X |
| 33 | K, I, V, C | Y, H | X | | X |
| 34 | G, R, E | V, A, W, Y | X | | X |
| 35 | Q | V | X | | |
| 36 | P, G | S, A, Q, R | X | | |
| 37 | T | | X | | |
| 38 | V, C | | X | | |
| 39 | S | F | X | | |
| 40 | R | D, A | X | | |
| 41 | T, Y | W, F | | | |
| 42 | E | G, R, I | X | | |
| 43 | R | N, T, L, H, V, I | X | | |
| 44 | G | I, V, S, A | | | X |
| 45 | R | V | | | X |
| 46 | R | Y | X | | X |
| 47 | W, N, R, L | K, T, S, Q, Y, C, M, H, V | | | X |
| 48 | L | V, M, I, P, G, C, S | | | X |
| 49 | R, A, F | K, G, S, C, Q, M, N, T, H | | | X |
| 50 | K, P, H | Y, R, G, V, N, F, A, S, T | | | |
| 51 | C, M, L, W, I, S, A, | K, R, H, T, V, G, P, Q, F, | | | |
| | N, E | Y, D | | | |
| 52 | I, V | R, T, G, Q, S, L, K, N, M, | | | |
| | | W, Y, F, C, A, H, E | | | |
| 53 | Q | V | | | |
| 54 | K, E, Q, S, M, P | R, V, L, C, Y, T, H, F, W, | | | |
| | | G, A | | | |
| 55 | R | C, H, K | | | |
| 56 | I, V | L | | | |
| 57 | L, Y, C | V, K, T, F, R | | | |
| 58 | A | E, Y, D, G, F, Q, C, T, L, | | | |
| | | V, R, N, I, K | | | |
| 59 | F, R, E | Y, H, I, K, M, L, W, A, T, | | X | |
| | | Q, C, N, D, V | | | |
| 60 | I, L, M | F, C, S, V | | | |

TABLE 9
DBP056 PPPTPFEVAGARIKEERAKLGLTQAELAKVAGVGQATVSRIEKGRKCSWELIEKIFEALKKV
(SEQ ID NO: 30)

| pos- | | | at_ | protein_ | loop/ |
|---|---|---|---|---|---|
| ition | best | tolerable | interface | core | insertion |
| 1 | M, W, D, F, H, E, T, Q, P, S, K, | G, R, L, A | | | |
| | C, I, N, V | | | | |
| 2 | K, L, F, P, N, Q, T, G, Y | W | | | |
| 3 | Y, W | K, T, S, E, D, P | | | |
| 4 | Q | P, R, C, A, S, D, | | | |
| | | V, T | | | |
| 5 | R, W | S, Y, C, V, L, M, | | | |
| | | P, I | | | |
| 6 | Q, C, K, M, R, D, F, N, L, A, I | S, P, G, W, Y | | | |
| 7 | P, G, A, S, R, Y, F, E, N, V, L, | I | | | |
| | W, H, C, M, D, K, T, Q | | | | |
| 8 | W, K, L, H, V, E, A, M, C, G, D, | | | | |
| | N, P, S, I | | | | |
| 9 | I, H, K, Y, P, Q, R, F, A, C, T, | M, L | | | |
| | E, N, W | | | | |
| 10 | R, M, S, C, G | F, A | | | |
| 11 | H, Y, K, N, A, G, F, V, R, S | T, C, P, L, W | | | |
| 12 | L, I, M, R, N, A | G, S, V, Q, W, C, T | | | |
| 13 | L, I, V | M, C | | X | |
| 14 | S, K, G, L, Q, M | | | | |
| 15 | T, M, A, S, N, K, I, V, Q, E, H, | | | | |
| | W, F, R, Y | | | | |
| 16 | G, L, R, V, T, K, N, E, A, Q, W, | F, H | | | |
| | I, Y | | | | |
| 17 | R | | | | |
| 18 | G, R, K, V, N | Y, A | | | |
| 19 | H, Y, K, T, I, M, N, G, V, R | C, E, F, L | | | |
| 20 | W, F | Q, K, H, C, L, S | | | |
| 21 | K, H, R | T, L, C, Q, V, W, | | | X |
| | | Y, D, G | | | |
| 22 | Y, V, I, L, R, F, M | A, K | | | X |
| 23 | S, K, T, A, M, P | G, R | X | | X |
| 24 | Q | | X | | |
| 25 | H, S, F, R, N, T, A, Q, L, M, V, | I, W, Y, C | X | | |
| | K, P | | | | |
| 26 | A, W | K, I, V, T, N, H, | | | |
| | | Q, D, M, E, S, Y, R | | | |
| 27 | V, L, M | | | | |
| 28 | G, S, A | | X | | |
| 29 | Q, K, R, G | V, W | | | |
| 30 | A, K, W, V, L, E, S, M, R, Q, N, | D | | | |
| | Y, C, G, F | | | | |
| 31 | G, V, A, T | M, C, L | | X | |
| 32 | V, K, F, G, R, H | I, A, L, Q, M, C, | X | | X |
| | | T, W, Y, E | | | |
| 33 | F, L, V, I, C, W | T | X | | X |
| 34 | S, H, N | G | X | | X |
| 35 | Q | | X | | |
| 36 | S | A | X | | |
| 37 | T, S | | X | | |
| 38 | I, V | | | X | |
| 39 | S | | X | | |
| 40 | R | | X | | |
| 41 | Y, A, I, F, V, S, T, C, E | | | X | |
| 42 | E | | | | |
| 43 | A, S, K, N, F, R, H, M | G, Q, Y, W, I | X | | X |
| 44 | Q, G | R, H, S | | | X |
| 45 | A, N, K, R | C, S, V, Y, M | | | X |
| | | | | | X |
| 46 | R, M, K | V, I, Q, A, P, N, | | | X |
| | | G, S | | | |
| 47 | L, N, S, M, T, C, I, V, A, H, | W, F | X | | X |
| | R, G | | | | |
| 48 | A, Y, C, S, T, G, R | M, N, W | X | | X |
| 49 | Q, K, Y, W, R, F, D, P, H, M | S, C, E, A, I, L | | | |
| 50 | I, H, F, Y, P, E, K, T, Q, S, D, | | | | |
| | W, N, R, G, A, M, C, V, L | | | | |
| 51 | P, G, V, T, C, L, A, S, M | I, F, N | X | | |
| 52 | R, K, F, M, Y, I, V, L | A, C | | | |
| 53 | D, I, V, Y, Q, K, R, A | W, E, G | | | |
| 54 | Q, N, K, Y, M, S | C, W, F, T | | | |
| 55 | V, I | A, M, L, T | | X | |
| 56 | E, M, N, V, S, F, Y, H, G, R, C, | Q, D | | | |
| | A, T | | | | |
| 57 | W, Q, S, R, Y, T, L, M, K | A, G, H, I, C, E, N | | | |
| 58 | Y, H, N, A, S, E | R, Q, M, K | | X | |
| 59 | F, I, C, L, A, Y | V, T | | | |
| 60 | P, Q, W, H, F, V, K, L, R | Y, M, T | | | |
| 61 | C, S, M, K, I, G, Q, V | A, H, D, F, W | | | |
| 62 | T, N, C, L, S, F, I, V, M, Q, K | W, E, D | | | |

TABLE 10
DBP057 MVLTPMERIGEFIKRARREAGLTQRELAELAGVGQSTVSRIEKGEKCSPELVEKILEALRKV
(SEQ ID NO: 31)

| pos- | | | at_ | protein_ | loop/ |
|---|---|---|---|---|---|
| ition | best | tolerable | interface | core | insertion |
| 1 | C, K, M | D, R, T, P | | | |
| 2 | E, V | C, T | | | |
| 3 | R, L, V | K, T, Y, C, H, S | | | |
| 4 | R, T | A, H, K, S, M, Y, E, C, N, P | | | |
| 5 | I, K, P | W, A, R, M, S, C, T, L, N | | | |
| 6 | K, M | R, P, Y, F, E, D | | | |
| 7 | P, K, R, Y, N, | V, I, T, Q, L, G, W, F, C, H, A | | | |
| | M, E | | | | |
| 8 | G, R, Y, N | H, L | | | |
| 9 | R | K, P, C, Y, I, E, A | | | |
| 10 | S, G, P | N, K, F, Y | | | |
| 11 | Q, S, E | P, G, R, T, A, C, W, M, K, I, | | | |
| | | H, Y, V, F, N, L | | | |
| 12 | K, R, F | V, M, Y, Q, A | | | |
| 13 | I | M, L, G, Y | | X | |
| 14 | K, I | H, C, S | | | |
| 15 | R | E, P, W, K, C, L, Q | | | |
| 16 | Y, A, F | K, V, N, H, S, T, I, L | | | |
| 17 | R | | | | |
| 18 | K, R, W | | | | |
| 19 | S, F, E, N | A, G, R, T, C, K, Q | | | |
| 20 | V | R, K, H, A, W | | | |
| 21 | K, A | H, S, G, N, R, P | | | X |
| 22 | K, L, H | R, M, G | | | X |
| 23 | K, T, F, P | R, V, L, H, I | X | | X |
| 24 | Q, V | N, F | X | | |
| 25 | R | S, L, D, K | X | | |
| 26 | N, M, T, K | I, V, Q, S, R, A, H, G, W, F, | | | |
| | | Y, L, E, P, D | | | |
| 27 | I, L | G | | | |
| 28 | A | P | X | | |
| 29 | M, K, E | T, L, I, H, W, A, Q, V, F, C, | | | |
| | | R, N, S, G, P | | | |
| 30 | K, R, Y, L, M | Q, N, H, I | | | |
| 31 | A, Q | I, C | | X | |
| 32 | R, G | K, H, C, T, W | | | X |
| 33 | C | V, R, P, M, S | X | | X |
| 34 | G | S | X | | X |
| 35 | Q | E | X | | |
| 36 | S | Q | X | | |
| 37 | T, L | S | X | | |
| 38 | V | I, S, T | | X | |
| 39 | S, F | | X | | |
| 40 | R | W | X | | |
| 41 | Y | C, H, T, A, I, K, M | | | |
| 42 | E | S | | | |
| 43 | H, K | R, D | X | | |
| 44 | C, R, G | W, T, H, F, D, N | | | |
| 45 | S, H | F, T, K, R, V, I, P, C, L, N, | | | X |
| | | M, A, Y, Q, W, G, D, E | | | |
| 46 | T, K | R, I, S, H, M, V, A, G | X | | X |
| 47 | Y, H, C | A, K, R, S, P, W, G, Q | X | | X |
| 48 | K, R, S | G | X | | X |
| 49 | F, W, P | Y, K, R, H, M, C, L, I, E, D | | | |
| 50 | T, R, K, F, C, | I, P, Y, Q, M, S, L, V, W, A, G | | | |
| | N, H, E | | | | |
| 51 | C, T, R, L | I, S, K, V | X | | |
| 52 | M, L, K, V, S | R, C, G, F, A, Q, E, T | | | |
| 53 | C, I, E | M, V, R, L, F, K, W, Y, H, Q, | | | |
| | | A, T, G, S, P, D | | | |
| 54 | W, K, P | R, G | | | |
| 55 | L, I | | | X | |
| 56 | R, L, E | K, W, G | | | |
| 57 | K, E | R, C, S, I, N, G, H, T, D, W, | | | |
| | | F, Q, L, M, A, Y | | | |
| 58 | Q, A, E | M, T | | X | |
| 59 | G, L | Q, T, V, F, C | | | |
| 60 | A, R | M, Q, D, T | | | |
| 61 | K | R, M, W | | | |
| 62 | R, V, D | H, P, S | | X | |

TABLE 11
DBP060 DIEKIAKAVKELREELGLTQAEFAKKIGIGQGTLSRFEKGGVLSPKTMERLLKALEKEFGFDVKK
(SEQ ID NO: 34)

| pos- | | | at_ | protein_ | loop/ |
|---|---|---|---|---|---|
| ition | best | tolerable | interface | core | insertion |
| 1 | K | R, P, T, G, M, S, W, A, Q, D, V, H, E, | | | |
| | | C, L | | | |
| 2 | R, K | I, D, Y, S, P, N, C | | | |
| 3 | K | P, M, R, F, Q, N, E, C, H, Y, V, D, I, | | | |
| | | S | | | |
| 4 | K | T, W, F, G, P, H, V, M, E, Q, C | | | |
| 5 | I | H, N, Q, T, M, L, D, Y | | | |
| 6 | N | A, E, V, S, M, K, D, L | | | |
| 7 | K | D, W, C, V | | | |
| 8 | A | M, R, F, Q, C, W, K | | | |
| 9 | C | A, V, M, S | | | |
| 10 | K | H, R | | | |
| 11 | F, R | S, E, K, W, D, Q, A, V, P, H, T | | | |
| 12 | L | K, I, E, Q | | | |
| 13 | R | | | | |
| 14 | V | I, R, A, Q, M, N, L, T, H, S, K, G, Y, | | | |
| | | C, F, W, E, P | | | |
| 15 | R | K, E, W, S, Q, Y, G, T, V, H, F | | | |
| 16 | L | F, W, I, T, V, E, H, M | | | |
| 17 | K | H, G, L, R, T, V, E | | | X |
| 18 | R, K | M, L, F, I, D, Y, T | | | X |
| 19 | K | R, T, M, W, F, G, I | X | | X |
| 20 | Q | | X | | |
| 21 | K | R, V, A, T, I, Q, W, L, M | X | | |
| 22 | N | Y, S, V, P, Q, K, M, A, T, W, H, F, C, | | | |
| | | I, G, L, E, R | | | |
| 23 | M | I, F, V, A, S | | | |
| 24 | A | F, G, E | X | | |
| 25 | K | N, F, C | | | |
| 26 | K | A, S, M, C | | | |
| 27 | G | N, I, W, R, C, H, F, V, T | | | |
| 28 | G | W, Q, M, A, F | | | X |
| 29 | I | | X | | X |
| 30 | G | V, T | X | | X |
| 31 | Q | | X | | |
| 32 | S | A, R, G, T | X | | |
| 33 | T | I | X | | |
| 34 | Q | L, M, C | | X | |
| 35 | S | | X | | |
| 36 | R | | X | | |
| 37 | F | M | | | |
| 38 | Q | L, E, M, I, P | | | |
| 39 | R | H, K, T, A | X | | X |
| 40 | H | R, K, W, G, V, I, F, Q | | | X |
| 41 | R | K, G, H, I, L, F, W, Y | | | X |
| 42 | K | R, H, V, F, I, P, D, S, G, M | | | X |
| 43 | L | C, S, K, P | X | | X |
| 44 | S | H, V | X | | X |
| 45 | K | R, P, A, W, C, V, G | | | |
| 46 | K | R | | | |
| 47 | T | L, M | X | | |
| 48 | M | F, W, L, I | | | |
| 49 | I | T, V, Y, S, W, H, K, R, Q, L, F, C, E, | | | |
| | | G, M, A, D, N | | | |
| 50 | R | I, K | | | |
| 51 | V | I, A, M, S, L, N, T, Q | | | |
| 52 | R, K | C, S, Q, H, G, L, V, W, T, Y, D | | | |
| 53 | K | V, Q, A, S, P | | | |
| 54 | R | N, A, S, F, C, L | | | |
| 55 | L | A, G, I, F | | | |
| 56 | K | R, I, E, N, T, W | | | |
| 57 | K | D, C, I, Y, F, M, Q, N | | | |
| 58 | K | R, Q, E, S, F, I, L, Y, M, D, G | | | |
| 59 | F | M, H, W | | | |
| 60 | G | L, C, F, Y, E, M, W, T | | | X |
| 61 | F | E, K, G, Q, Y, I | | | |
| 62 | R, H | V, D, M, T, K, G, W, Q, Y | | | |
| 63 | E, S | V, Y, N, T, F, H, W, I | | | |
| 64 | M | K, L, Y, E, D, P | | | |
| 65 | I | K, D, C, V, Q, G, S, A | | | |

TABLE 12
DBP062 KEELEKLLKIIESLPKKFREVIILKFVEGLSYTEIAERLGVSRGAVYSRLRSALKKIEEALKK
(SEQ ID NO: 36)

| posit- | | | at_ | protein_ | loop/ |
|---|---|---|---|---|---|
| ion | best | tolerable | interface | core | insertion |
| 1 | A, T, R, Q, K, C | P, M, V, L | | | |
| 2 | M, H, Q, A, V, S, K, E, T, N, D | P, L, Y, W, I, G | | | |
| 3 | A, Q, E, D | S, G, C, H | | | |
| 4 | W, V, L, F | M | | | |
| 5 | G, D, T, Q, C, E, N | A | | | |
| 6 | I, Y, S, K | F, H, R, L, G | | | |
| 7 | V, L | I | | | |
| 8 | I, V, L, C, Q | Y, A | | | |
| 9 | A, H, C, W, G, D, I, M, V, K, R | S, N | | | |
| 10 | V, I, L | | | | |
| 11 | I | | | | |
| 12 | H, M, D, N, E, T | I, L | | | |
| 13 | M, H, A, R, N, E, G, D, Q, S | T, L, K, F | | | |
| 14 | F, L, V | | | X | X |
| 15 | Q, P, H, T | G | | | X |
| 16 | E, R, G, A, I, P, Y, L, K, S, | | | | |
| | N, F, M, W, H, V, D, Q, T, C | | | | |
| 17 | A, M, K, H, R, N | Q, Y, T | X | | |
| 18 | M, Y, V, C, A, L, G, F, S, Q, | D, E | | | |
| | N, H, W | | | | |
| 19 | S, M, H, R, K | T, L, Q | | | |
| 20 | V, C, D, H, A, E, F | N, I, L, Q | | | |
| 21 | M, L, V, I, C | | | X | |
| 22 | F, V, I, L | | | | |
| 23 | L, F, Q, N, R, I, V, M | S, K, H, A, Y | | | |
| 24 | Q, Y, N, G, W, L, M, C | D | | | |
| 25 | N, R, M, K | T | | | |
| 26 | I, F, L | W | | | |
| 27 | L, V, K | G, T, R | | | |
| 28 | T, R, M, G, P, Y, N, H, E, Q, V | K, D, W, S, A | | | X |
| 29 | H, G | V | | | X |
| 30 | W, N, Q, K, L, A, F, D | V, I, H | | | X |
| 31 | K, S, T, A | R, N, Q, G, M, H | X | | X |
| 32 | W, G, A, Y, C, N | | X | | |
| 33 | G, F, M, Y, W, H, I, S, K, P, | A, E, C, R, D | X | | |
| | Q, T, V, L | | | | |
| 34 | M, S, C, L, E | D | | | |
| 35 | F, I, V | L, Q, M | | X | |
| 36 | E, A | C, T, S, G | | | |
| 37 | G, F, P, W, L, A, E | H, I, C, V | | | |
| 38 | T, E, K, Y, S, R, M, W | N, C | | | |
| 39 | P, K, V, F, M, Y, W, L, Q | N, I, D, A, C, G, | | | |
| | | S, T | | | |
| 40 | K, F, E, M, G, D, V, S, L, W | T, R | | | X |
| 41 | M, I, F, V, T, A | G, S | X | | X |
| 42 | C, S | G, K, R | X | | X |
| 43 | G, C, S, R | K, L, V, I | X | | |
| 44 | C, T, Q, S, G, V | P, N, F, H | X | | |
| 45 | T, S, D, A, R | I, V | X | | |
| 46 | F, E, V, A, L, I, C | R, M, T | X | | |
| 47 | N, F, Y | | X | | |
| 48 | C, S, Y, A, F | T | X | | |
| 49 | S, R, W | | X | | |
| 50 | W | M, Q, T, N, I, A, | | | |
| | | V, L, F, C, G | | | |
| 51 | M, S, W, R | | X | | |
| 52 | T, F, W, S | Q | X | | |
| 53 | D, A | | | X | |
| 54 | F, Y | I, V, N, S, M, L | | | |
| 55 | R, N, K | | | | |
| 56 | Q, Y, C, K, I | V, F | | | |
| 57 | K, I | | | | |
| 58 | R, A, E, Q, V | W, S, N, H | | | |
| 59 | G, Q, W, M, E, H, V, F | D, I, L, A, N, Y, | | | |
| | | S | | | |
| 60 | M, G, C, N, L, Q, E, A, S, P | F, Y, K, T | | | |
| 61 | V, I, L, M, F | C | | | |
| 62 | N, Q, H, K, R | | | | |
| 63 | R, V, Y, I, T, S, K, L, M, H, P | Q, E, F | | | |

TABLE 13
DBP069 MKEEGRKLKELRERLGLTQAELAEALGLGQSTISRLERGRKEISPEVWEKALALLE
(SEQ ID NO: 38)

| pos- | | | at_ | protein_ | loop/ |
|---|---|---|---|---|---|
| ition | best | tolerable | interface | core | insertion |
| 1 | R, L, M, T, P, F, N, H, D, | Y | | | |
| | V, K, I, W, G, S, C, Q, E | | | | |
| 2 | K, W, A | H, D, G, L | | | |
| 3 | I, P | T, R, L, V, S, H, G, Y, | | | |
| | | M, N, C, A, E, Q, K | | | |
| 4 | R, K, P, S, L, C | H, N, Y, E, G, A, M, T, | | | |
| | | Q | | | |
| 5 | G, T, K, Y, F | Q | | | |
| 6 | R, I, S | H, W, N, V, F | | | |
| 7 | A, K, S, M, R | C, Q, Y, W, G | | | |
| 8 | I, L, F, A, V | E, G | | X | |
| 9 | K | M, S, R, A | | | |
| 10 | A, H, C, G, R, T, I, M, S, | N | | | |
| | L, Q, Y, P, F, W, V, E, D, K | | | | |
| 11 | L, I, M | F, T, W, H, A, C | | | |
| 12 | R | W | | | |
| 13 | F, C, V, A, S, N | Y, L, I, T, Q, G, M, H, | | | |
| | | R, K, W, D, E | | | |
| 14 | D, R, I, F, T, N, V, Q, L, | K, M | | | |
| | Y, W, G, H | | | | |
| 15 | L, P, A | Q, T, H, V, Y, W, M | | | |
| 16 | K, A | R, G, C, V, H, I, Y | | | X |
| 17 | K, F, M, C, N | W, L, H, I, V | | | X |
| 18 | K | R, T, S, V, I | X | | X |
| 19 | Q | | X | | |
| 20 | R, K | N, A, T, W, V, G, S | X | | |
| 21 | K, R, A, L, V, Q, S, I, T, | H, G, C, F, Y, E, W | | | |
| | N, M | | | | |
| 22 | L, I | F, P | | | |
| 23 | G, A | Y, T, I, L | X | | |
| 24 | R | K, H, P, Q, S, F, Y, T, | | | |
| | | M, N, L, G, A, I, V, E, | | | |
| | | W, C | | | |
| 25 | K, R, F, N, L | H, V, A, T, E, G, P, W | | | |
| 26 | L | D, K, W | | | |
| 27 | L, G, M, H, R, S | K, E | | | X |
| 28 | C | F, K, V, L, T, Y, I, G, | | | X |
| | | S | | | |
| 29 | S | G, W | X | | X |
| 30 | Q, R | | X | | |
| 31 | S, W | Q, V, T, A | X | | |
| 32 | T | R, G | X | | |
| 33 | I, V | R | X | | |
| 34 | W, S, I | | X | | |
| 35 | R | Y | X | | |
| 36 | L | W, Y | | | |
| 37 | E | M, V, W, D, A, Q | | | |
| 38 | R, E | P, W, S, M | X | | |
| 39 | G | T, P, S, C | | | X |
| 40 | R | | | | X |
| 41 | R | K, M, F, T | X | | X |
| 42 | K | S, L, Y, A, H, R, G, F, | | | X |
| | | N, T, P, M, V, W, Q, C, | | | |
| | | I, E | | | |
| 43 | I, L | P, W, F, G, Q, A | | | X |
| 44 | R, V, S, K | H, Q, C, F, M, W | | | X |
| 45 | C, P, I, W, R, K, Y, S, Q, | D, L, N, T, E | | | |
| | H, G | | | | |
| 46 | L, Y, Q, W, F, S, T, I, A, | M, H, V, E, G | | | |
| | K | | | | |
| 47 | V, N, L, T, K, I, R, F, W, | S | | | |
| | Y | | | | |
| 48 | M, W, F | S, K, R | | | |
| 49 | F, R, C, E, A, L, K, D, M, | P | | | |
| | T, N, V, Q, I, Y | | | | |
| 50 | K | H, E, P, D, A | | | |
| 51 | V, A, T, S | N, W, I | | | |
| 52 | L, M, E, W, G, Y, R, C, F | K, D, T, Q, H | | | |
| 53 | Y, K | A, L, I | | | |
| 54 | L, I | T, M, R | | | |
| 55 | L, I, V | K, Y, Q | | | |
| 56 | E, Q, S, H, W, K, D, L, N, | G | | | |
| | T | | | | |

TABLE 14
DBP085 TLSQLTPQEMRIARLASEGMPNREIATRLFISPRTVEWHLRRAMRKLGVRNRTQMARRIDTRL
(SEQ ID NO: 44)

| pos- | | | at_ | protein_ | loop/ |
|---|---|---|---|---|---|
| ition | best | tolerable | interface | core | insertion |
| 1 | V, R | G, K, T, L, N, Q, F, H, C, M, Y, E, I, | | | |
| | | S | | | |
| 2 | Q, R | L, K, H, N, W, E, M, I, G, Y | | | |
| 3 | S, K | W, F, H, C, E, G, R, D, I, P, N, A, V | | | |
| 4 | R | Q, K, M, A, W, S, T, N, C, V, G, I, P, | | | |
| | | L, H | | | |
| 5 | L | | | | X |
| 6 | T | | X | | X |
| 7 | N | K, P, Y, A, R, S, T, Q, H, W, F | | | |
| 8 | Q | R, S, Y, K | X | | |
| 9 | E | Q | X | | |
| 10 | Y | M, L, A, K, V, E, G, R, I, S, F | | | |
| 11 | R | | | | |
| 12 | V | I | | X | |
| 13 | W | A, M, F, I, C | | X | |
| 14 | H | R, S, L, V, Y, K | | | |
| 15 | L | T, F, W, Y | | | |
| 16 | W | Y, S, A, T, I, V, F, K, C | | | |
| 17 | A | S, T, F, I, L, C, K, Q, G, R | | | |
| 18 | M | T, G, A, L, Q, V, S, E, F, D, N, Y, R, | | | X |
| | | H | | | |
| 19 | G | L | | | X |
| 20 | M | Y, Q, L, V, W, T, R, I, K, H | | | X |
| 21 | G | P, E, R, D, F, H, Q, L, T, V, A | X | | X |
| 22 | N | T, L, Y, V | X | | |
| 23 | R | C, T, K, L | X | | |
| 24 | E | N, Y, D, F, A, I, C, V, M, L, S | | | |
| 25 | I | F, Q | | X | |
| 26 | A | L, S, R | | | |
| 27 | A | Q, Y, M, V, H, T, K, C, E, G, S, R, I, | | | |
| | | N | | | |
| 28 | Y | R, M, N, H, L, V, S | | | |
| 29 | L | Y | | | X |
| 30 | S | L, F, R, W, V, Y, N, T, C, H, K, I, A, | | | X |
| | | E, G | | | |
| 31 | I | R, L | X | | X |
| 32 | S | Q, H, M, R, A | X | | X |
| 33 | P | M, F, I, Y, S, V, T, H, Q | X | | |
| 34 | R | M, K, Y | X | | |
| 35 | T | R | X | | |
| 36 | V | R | | X | |
| 37 | E | I, G, M, V, S | X | | |
| 38 | W | A | X | | |
| 39 | H | C, M | X | | |
| 40 | L | M | X | | |
| 41 | R | | X | | |
| 42 | R | S, W, D, N | X | | |
| 43 | A | P, S, L | | X | |
| 44 | M | P, Y, R | | | |
| 45 | R | K | | | |
| 46 | K | A | | | |
| 47 | L | | | X | |
| 48 | G | K, C, N, R | | | X |
| 49 | A | V, F | | X | X |
| 50 | R | T, C, K, Q, L | | | X |
| 51 | T | N, F, V, G, Q, K, Y, S | | | X |
| 52 | R | G, V, K | X | | |
| 53 | W | A, Y, F, R, L, N, T, Q, M, V, C, S, K | | | |
| 54 | F | Q, T, G, C, Y, H, S, N, A, M, F, D | | | |
| 55 | L | M, A, V, I, F, N | | | |
| 56 | A | T, K, Y, Q, I, V, W, M, R, L, C, S | | | |
| 57 | Y | Q, E, R, M, I, S, N, L, H, A, G | | | |
| 58 | A | M, K, I, Y, C, R, L, F, W, Q, H, N, E, | | | |
| | | V | | | |
| 59 | L | I, F, W, Q, T, G, V, Y, H, M, C, A | | X | |
| 60 | G | W, R, Q, D, H, S, L, K, N, E, I, Y, C, | | | |
| | | P, A, V, M | | | |
| 61 | L | Y, E, Q, H, S, A, T, F, P, N, I, V, M, | | | |
| | | D | | | |
| 62 | Y | F, M, L, I, R, A, N, K, S, D, Q, H, V, | | | |
| | | T, C | | | |
| 63 | N | T, L, F, G, D, H, P, A, Q, C, M, W, I, | | | |
| | | E, R, Y | | | |

TABLE 15
DBP090 NKYQLSLLESAFQSNRYPDISQRATLASQTGLPERRIKIWFQNRRQRWKRKK (SEQ ID NO: 49)

| pos- | | | at_ | protein_ | loop/ |
|---|---|---|---|---|---|
| ition | best | tolerable | interface | core | insertion |
| 1 | N, G, P, D | | | | |
| 2 | F, K, P, G, Y, H, E | | | | |
| 3 | Y, R, P, G, M, N, V, T, E, L, F, | | | | |
| | K, I, S, A, Q, H | | | | |
| 4 | E, G, R | S, Q, P | | | |
| 5 | G, A, T, V | D, H, M, L, R, Q, S, | X | | |
| | | P | | | |
| 6 | L, P, S, T, K, I, N, G, V, H, R, | | | | |
| | E | | | | |
| 7 | R, H, S, P, E, M, T, G, Q, F, K, | L, Y, N | | | |
| | A | | | | |
| 8 | P, N, G, M, T, W, S, K, V, A | L, F, Y, I | | | |
| 9 | D, P, K, S, T, H | A, M, G, Q, Y, E, | | | |
| | | N, V, I | | | |
| 10 | P | S, G | | | |
| 11 | Q, N, C, K, E, G, P, S, H, D, T, | W, L, A | | | |
| | I, R | | | | |
| 12 | R, V, L, Q, P, G, N, Y, A | D, S, K, I, E, T, | | | |
| | | F, M | | | |
| 13 | P, V, T, K, H, I, S, R, Y, D | A, Q, W, N | | | |
| 14 | P, G | D, S | | | |
| 15 | P, I, E, T, H, R, M, G | V, K, Y, N, L, W | | | X |
| 16 | L, Y, T, V, S, G, N, E | M, R, D | | | X |
| 17 | T, S, G, H | Q, E, R, D, Y, M | X | | X |
| 18 | M, L, R, H, N, V, Y, Q, S, T, K | F, G, P | | X | X |
| 19 | Y, V, Q, R, K, F, S, H, E, P, W | A, T, L, D | | | X |
| 20 | T, P, I, R | | X | | |
| 21 | W, L, S, T, P, V, D, G, N, C | | | | |
| 22 | S, F, N, R, T, G, W, P, H, Y, V, | L, Q, E | | | |
| | I, M | | | | |
| 23 | E, S, K | R, P | | | |
| 24 | G, W, P, D, S, T, Y, V, H | A | | | |
| 25 | G | T, P | | | |
| 26 | H, E, R, I, N, Y, G, V, A, F, P, | M, K, S, L, C | | | |
| | Q, T, W, D | | | | |
| 27 | Y, N, T, I, V, H | P, E, D, C, A | | | |
| 28 | P, S | I | | | |
| 29 | H, A, S, V, P | Q | | | |
| 30 | N, K, T, Q, P, S, D, H, G, E, R, | | | | |
| | A, L | | | | |
| 31 | V, P, T, H, Q, L, I, S, W | A, G | | | X |
| 32 | P, A, E | V, L, H, Q, R, S | | | X |
| 33 | P, R | | | | X |
| 34 | P, D | T, G, K, H, E, Q, N | | | |
| 35 | N, R, P, K, S | | | | |
| 36 | K, R, P | | X | | |
| 37 | N, K, S, H, A, T, Q, R, F, G, Y, | L, P, M, E, D, I | | X | |
| | W, V | | | | |
| 38 | P | G, N, K, Q, R | X | | |
| 39 | P, Y, H, N, G, W, T, A | S, V, F, M, I, K | X | | |
| 40 | F, N | P, R, S, W, Y, Q | X | | |
| 41 | M, G, W, A, S, Y, V | Q, R, P, L, N, F, | | | |
| | | T, H, D, E | | | |
| 42 | G | L, P, Q, T, M | X | | |
| 43 | A, G, P, N, T, H | | X | | |
| 44 | P, Y, N, C | R | X | | |
| 45 | G, A, R, N, H, P, S | C | X | | |
| 46 | Y | W, F, Q | X | | |
| 47 | S, Y, R, H, P, W | Q | X | | |
| 48 | W, P, F, Y | | | | |
| 49 | R, P, T, H, A | S, G, Q, K | X | | |
| 50 | R, N | | X | | |
| 51 | K, P, R | | | | |
| 52 | A, S, K, P, G, R, Q, Y, M, T | | | | |

TABLE 16
DBP001 TARELEVAALIAQGRSNREIAEELNISERTVERYVRRILRKLGLRNRAQIAAWVIRRS
(SEQ ID NO: 1)

| pos- | | | at_ | protein_ | loop/ |
|---|---|---|---|---|---|
| ition | best | tolerable | interface | core | insertion |
| 1 | T | | | | |
| 2 | A, L, M, T, K, H | | | | |
| 3 | R | P | X | | |
| 4 | E | G | | | |
| 5 | L, V, Q, R, K | | | | |
| 6 | E, Q, T | | | | |
| 7 | V, G | | | X | |
| 8 | A, I, V, C, M, S | | | | |
| 9 | A, G, N, T, Y, E, R, D | | | | |
| 10 | L, K, G, N, A, R, H, I | | | X | |
| 11 | I, V | A, Y, L, M | | X | |
| 12 | A, Q, K, G | | | | |
| 13 | Q, N, Y, F, K, G, A, D, C, E, M, | | | | X |
| | T | | | | |
| 14 | G, L, E, D, Q, S, R, I, V, T, M, | | | | X |
| | A, H | | | | |
| 15 | R, V, S, C, L, K, Q, M, H | | | | X |
| 16 | S, W, L, N, K, T, Y, V | | X | | X |
| 17 | N | F | X | | |
| 18 | R, T, Q | | X | | |
| 19 | E, I, W, R, Y, A, D, F, K, Q, L, | | | | |
| | V, G, T | | | | |
| 20 | I, C, A | | | | |
| 21 | A, G | C | | | |
| 22 | S, E, M | | | | |
| 23 | E, D, Q, S, R, N, C, L, M, T, K | | | | |
| 24 | L | C | | | X |
| 25 | N, R, T, L, G, D, M, Y, Q, I, V, | | | | X |
| | F, S | | | | |
| 26 | I | F | X | | X |
| 27 | S, Q, A, M, K, L, H | | X | | X |
| 28 | E, T, V | | X | | |
| 29 | R, K, L, W, I | C | X | | |
| 30 | T | | X | | |
| 31 | V | | | X | |
| 32 | E, D, W | H | X | | |
| 33 | R | P | X | | |
| 34 | Y, A | | | | |
| 35 | V, I | W | X | | |
| 36 | R, K, V | | X | | |
| 37 | R | F | | | |
| 38 | I, L | V | | X | |
| 39 | L, M, K, C, Q, R, I | | | | |
| 40 | R, K | | | | |
| 41 | K | | | | |
| 42 | L, A, K, V, N | | | | |
| 43 | G, A, Q, H, N | | | | X |
| 44 | L | | | X | X |
| 45 | R | | | | X |
| 46 | N, W, T, H, L, G, A | | X | | X |
| 47 | R | | X | | |
| 48 | A, K, W, M, Q, E, N, Y, T, R | | | | |
| 49 | Q, H, A | | | | |
| 50 | I, L | | | X | |
| 51 | A, K, V, P, R, G, S | D | | | |
| 52 | A, K, T, W, E, V, M, S, R | | | | |
| 53 | W, G, E, R, Q, L, S | | | | |
| 54 | V, T, Y, F, M, S | N | | X | |
| 55 | I, N, A, S, M, Q, R, K | | | | |
| 56 | R, S, T, F, E, Q, M, A | | | | |
| 57 | R, Q, H, I, A, M, N, T, K, Y | | | | |
| 58 | S, F, D, T, Q, K, R, G, P, N | | | | |

TABLE 17
DBP003 TKREREVLKLIAEDYGNKEIANRLNISERTVERYIRRILRKLGLKNRAELVRYAIRHG
(SEQ ID NO: 2)

| pos- | | | at_ | protein_ | loop/ |
|---|---|---|---|---|---|
| ition | best | tolerable | interface | core | insertion |
| 1 | T, P, S | | X | | |
| 2 | K, P, H | | | | |
| 3 | R, T | | X | | |
| 4 | E | W | | | |
| 5 | R, L, M, Q, G, N, W, K, S | H | | | |
| 6 | E, D | | | | |
| 7 | V | M | | X | |
| 8 | L, M, V | | | | |
| 9 | K, E, T, G, D, Y, S, R, M, Q, C, | | | | |
| | H, N, L, V | | | | |
| 10 | L, Y, W, E, F | | | X | |
| 11 | I, A, F, M, L | | | | |
| 12 | A | F | | | |
| 13 | E, S, Q, N, H, F, C, Y | | | | X |
| 14 | D, S, K, T, A, E, F, H, L, V, C | | | | X |
| 15 | Y, R, H, Q, M, T, W, P, L, D, C, | | | | X |
| | A, N | | | | |
| 16 | G, E, W, R, A, C, L, I, N, H, M | | X | | X |
| 17 | N, P, L | | X | | |
| 18 | K, L, Q, T, S | | X | | |
| 19 | E, W, G, P, R, S, I, Q, N, T, V, | | | | |
| | Y, L | | | | |
| 20 | I | | | | |
| 21 | A, G | C | | | |
| 22 | N, W, Y, A, G, M, K, E, F, D, T, | | | | |
| | L, Q, I | | | | |
| 23 | R, V, W, M, E, C, I, S, F, G, D, | | | | |
| | N, A | | | | |
| 24 | L, F | | | | X |
| 25 | N, K, S, T, H, M, G | | | | X |
| 26 | I | C | X | | X |
| 27 | S, M, N, T, V, G, H, L, A, K | | X | | X |
| 28 | E, S, V, A, W, C, Q, I | | X | | |
| 29 | R, L, I, M, K, W | A | X | | |
| 30 | T, C | | X | | |
| 31 | V, I | G | | X | |
| 32 | E, W | | X | | |
| 33 | R, Q | | X | | |
| 34 | Y | R | X | | |
| 35 | I, L | N | X | | |
| 36 | R, I, F, S | E | X | | |
| 37 | R, F, S, A, M | L | X | | |
| 38 | I | A, S | | X | |
| 39 | L, R, N, T, F | S | X | | |
| 40 | R, Q, K, M, Y, W, G, F, V, S, C | | | | |
| 41 | K | | | | |
| 42 | L, F, V, T | | | | |
| 43 | G, W, S, N, A, H, R | | | | X |
| 44 | L, M, I | | | X | X |
| 45 | K, V | | | | X |
| 46 | N, R, C, G, S, T | X | | | X |
| 47 | R, Y, W | X | | | |
| 48 | A, T, R, M, W, Q, V, P, C, H, S | | | | |
| 49 | E, R, S, M, V, Q, T, A | | | | |
| 50 | L | | | | |
| 51 | V, I, A, N, S, G, C, K, E, L, M, | | | | |
| | R, Y | | | | |
| 52 | R, I, P, H, K, A, Y | | | | |
| 53 | Y, W, F, H | | | | |
| 54 | A, W, Y, T, H, S L | | X | | |
| 55 | I, R, T, L, M, S, F, H, E, C, V, | | | | |
| | Y, D, K | | | | |
| 56 | R, K, F, G, D, W, H, M | | | | |
| 57 | H, S, P, Y, D, W, L, E, R | | | | |
| 58 | G, T, H, L, E, D, C, V, S, P, K, R | | X | | |

TABLE 18
DBP006 DWAARAAAARRLRKERGLTQAELGELAGVSRTTVSRIELGRPDVSQASVDAVLAVL (SEQ ID NO: 4)

| pos- | | | at_ | protein_ | loop/ |
|---|---|---|---|---|---|
| ition | best | tolerable | interface | core | insertion |
| 1 | R, F, M, H | K, P, Q, W, N, S, T, L, Y, G, | | | |
| | | I, V, E, D | | | |
| 2 | R, G | K, I, V, L, T, P, M, Y, N, A, | | | |
| | | W, H, S, Q, C | | | |
| 3 | K | R, C, W, M, Q, I, T, S, F, Y, | | | |
| | | A, H, V, L, N, G, P, E, D | | | |
| 4 | K, V, F | Q, T, N, A, W, I, H, M, S, Y, | | | |
| | | D, P, E, C | | | |
| 5 | R, M | F, T, K, Q, V, Y, I, L, C, W, | | | |
| | | N, H, S, A | | | |
| 6 | G, R | A, K, V, C, I, M, Y, T | | | |
| 7 | K | R, A, V, I, T, H, L, M, N, Y, | | | |
| | | G, F, W, C, E, D | | | |
| 8 | W, M, N, Y, F, K, T, | S, A, D, G, V, C | | | |
| | L, I, E, Q | | | | |
| 9 | M, V, C, F | A, I, Y, W, S | | X | |
| 10 | R | K | | | |
| 11 | R, K, F | A, S, M, T, H, Y, C, L, N | | | |
| 12 | K, M, A, L, R | I, W, V, Q, T, S | | X | |
| 13 | R | | | | |
| 14 | K | | | | |
| 15 | A, Q, L, M | E, I, S, C, N, F, V, H, W, D, | | | |
| | | Y, T, R, G, K | | | |
| 16 | R | L, K, M, A, Q, W, F, S | | | |
| 17 | G | K, R, C | | | X |
| 18 | L, W | M, Y, F | | | X |
| 19 | T | S | X | | X |
| 20 | Q | | X | | |
| 21 | K, R | A, S, Q, T, M, H, V, F | X | | |
| 22 | Q, K, M | R, E, G, D, H, V, T, S, C, N | | | |
| 23 | V, L | I | | X | |
| 24 | G | | | | |
| 25 | K, A, L, M, H, F, | R, Q, Y, C, I, E, G, D | | | |
| | V, T | | | | |
| 26 | L, M | K, R, I | | | |
| 27 | A | V | | X | |
| 28 | G | H | | | X |
| 29 | V | L, I | X | | X |
| 30 | S | | X | | X |
| 31 | R | | X | | |
| 32 | T | G, A, N | X | | |
| 33 | T | V | X | | |
| 34 | V | I | | X | |
| 35 | S | W, G, Y | X | | |
| 36 | R | | X | | |
| 37 | I | F, V, L | | X | |
| 38 | F | | | | |
| 39 | R, N, H, L | K, M, F, Q, W, A | X | | |
| 40 | G | | | | X |
| 41 | K | R, M, S | X | | X |
| 42 | K, G, V, H, R, S, | T, Y, Q, A, M, P, N, W, L, C, | | | X |
| | F, I | D, E | | | |
| 43 | G, N | Y, W, F, D, E, H, P, C | X | | X |
| 44 | V | | X | | X |
| 45 | S | G | X | | X |
| 46 | K | C, Q, A, L, W, I, S, E, H, F | | | |
| 47 | K, F, L | Q, A, H, N, S, T, C, M, Y, W, | X | | |
| | | G, V, I | | | |
| 48 | K | S, A, L, M, V | X | | |
| 49 | I | V, R, Y, F, M | | | |
| 50 | N, Q, Y | R, M, D, K, E, F, W, H, A, S, | | | |
| | | C, I | | | |
| 51 | A | M, S, V, Q | | | |
| 52 | V | I | | X | |
| 53 | W, M, L, I | Y | | | |
| 54 | S, R, K, A, N, G, H, | L, Y, C, V | | | |
| | M, F, W, T, E, D, I, | | | | |
| | Q, P | | | | |
| 55 | A, V, I, W | C | | X | |
| 56 | L | F | | | |

TABLE 19
DBP035 GFGRAVKEKRKELGLTQKEFAEKAGLSRRTIIRIERGYIVPPKATKEKIAKALGTSVEELEQA
(SEQ ID NO: 15, and variants SEQ ID NO: 49-51)

| pos- | | | at_ | protein_ | loop/ |
|---|---|---|---|---|---|
| ition | best | tolerable | interface | core | insertion |
| 1 | N, G | P, M, E, F, V, I, A, K, T, | | | |
| | | Y, W, H | | | |
| 2 | I, Y, L, F, W, M, V | | | | |
| 3 | G, Q | N, A, C, F, P, S | | | |
| 4 | V, S, M, Q, R | D, E, A, P, N, F, H, K, C, | | | |
| | | I, T | | | |
| 5 | V, I, Y, F, A | N, L, T, Q, C, G, E, H, M, | | X | |
| | | D, P, K, W | | | |
| 6 | I, V, T, M, C, F, S | | | | |
| 7 | R, K | W, Y | | | |
| 8 | D, E, F | C, I, L, T, W | | | |
| 9 | G, K | R, L, A, Q, W, Y, S, M, C, | | X | |
| | | N, F, I, V, D | | | |
| 10 | R | | | | |
| 11 | N, L, R, K | W, T, A, V, S, Q, C | | | |
| 12 | E, N, W, S, Y | M, H, L, V, G, F, C, I, K, | | | |
| | | D, A | | | |
| 13 | Q, A, L, H, F, R, M, G, E, | N, K | | | |
| | S, D, C, V, Y | | | | |
| 14 | G, M, E, V, T, L | D, C, Y, I, N | | | X |
| 15 | L, W, K, F, Y, Q | C, D, N, S | | | X |
| 16 | T | I | X | | X |
| 17 | | | X | | |
| 18 | V | I, L, Q, A, K, Y, F, S, M, | X | | |
| | | H | | | |
| 19 | T, E, I, C, V, L | A, M | | | |
| 20 | F, C | Y, L | | | |
| 21 | A | G, S | | | |
| 22 | E, D, W, C | Y, T, Q, G, L, I | | | |
| 23 | K, H | C, V, Q, S, N | | | |
| 24 | A, S | | | | |
| 25 | F, T, G | Y, K, S, R, C, W, A, L | | | X |
| 26 | R, L | | X | | X |
| 27 | S | | X | | X |
| 28 | R, V | | X | | |
| 29 | F, R | N | X | | |
| 30 | T | | X | | |
| 31 | I | | | X | |
| 32 | I | | X | | |
| 33 | N | K, R, S, Y, G, T, Q, I | X | | |
| 34 | Y, I | V | | | |
| 35 | E | | | | |
| 36 | R, N, S | K, Q | X | | |
| 37 | G, E | A, L, S, N, Q, H | | | X |
| 38 | Q, Y | P, N | | | X |
| 39 | H, I, S | V, Q, G | X | | X |
| 40 | N, V, E | T, G, I, M, L, D, Y | | | X |
| 41 | P | | X | | X |
| 42 | Q, M, F, S, T, I, A | K, P | X | | X |
| 43 | K | N, T | | | |
| 44 | K, R, A | G, V, P, S, Y, H, F, T, Q, | X | | |
| | | T | | | |
| 45 | T | | X | | |
| 46 | K | R | | | |
| 47 | M, G, F, S, T, Q, N, V, I, | Y, D, R, K | | | |
| | E, H | | | | |
| 48 | K | | | | |
| 49 | T, V, L, I, A, M, C | | | | |
| 50 | V, S, A, I, R, M | C, L, Y, T, G | | | |
| 51 | S, K, R, N, L, C, F, T, Y, | | | | |
| | V, I | | | | |
| 52 | G,A | S | | X | |
| 53 | C, I, S, T, A, L | F, G, Y, M | | X | |
| 54 | C, L, G, A, K, M, R, Q, S, | V, I, Y | | | X |
| | T, W, E, F, H, D | | | | |
| 55 | W, L, I, E, Q, M, V, T, R | F, Y, D, A, C, N | | | X |
| 56 | C, S, M | I, R, P, F, G, T, W, N, D, | | | X |
| | | Y, E, L, H | | | |
| 57 | R, K, V, T | I, L, P, C, Q, A, G, Y | | | |
| 58 | C, E | P, Y, K, M, H, T, Q, A, N, | | | |
| | | I, F, D, S, G | | | |
| 59 | Q, E, N | I, Y, G, W, T, K, M, H, R, | | | |
| | | L, C, V, S, A, P | | | |
| 60 | V, I, L | F, M, C | | X | |
| 61 | I, V, L, E, W, H | F, T, S, C, Q, N, M, R | | | |
| 62 | C, W, Q, V, K | L, M, H, R, I, N, T, G, D | | | |
| 63 | F, V, A, C, D, I, W | Y, G, H, R, P, S, N | | | |

In one embodiment of each of the above aspects, amino acid substitutions relative to the reference polypeptide or fusion polypeptide are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the disclosure provides fusion proteins, comprising
(a) the polypeptide of any embodiment or combination of embodiments herein; and
(b) one or more functional domains.

As noted above, the polypeptides may be used, for example, to modulate transcription in living cells; to edit specific DNA bases in a genome by fusion with a base editing domain; to nick or cleave DNA at specific sites by fusion with a nickase or nuclease domain; or to integrate DNA at specific sites in a genome by fusion with a recombinase or integrase domain. Thus, in certain embodiments, the one or more functional domains may be selected from the group consisting of a transcriptional effector domain, a multimerization scaffold protein, a nucleotide editing domain, a DNA methyltransferase domain, a nickase domain, a recombinase/integrase domain, another DNA binding polypeptide of the disclosure, and a nuclease.

In a further aspect, the present disclosure provides nucleic acids, including isolated nucleic acids, encoding the polypeptides and fusion proteins of the present disclosure. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the invention operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In other embodiments, the expression vector comprises an expression cassette, which can be chromosomally integrated into a host cell.

In a further aspect, the present disclosure provides host cells that comprise the expression vectors, polypeptides, fusion proteins, and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

The disclosure also provides kits, comprising the host cells, expression vectors, polypeptides, fusion proteins, and/or nucleic acids of any embodiment or combination of embodiments herein. In one embodiment, the kit comprises:
(a) a first expression vector comprising the nucleic acid of any embodiment or combination of embodiments herein, operatively linked to a promoter; and
(b) a second expression vector comprising a DNA target of the polypeptide expressed by the first expression vector.

In another embodiment, the kit comprises
(a) a first host cell comprising a chromosomally-integrated expression cassette comprising the nucleic acid of any embodiment or combination of embodiments herein, operatively linked to a promoter; and
(b) a second host cell comprising a chromosomally-integrated DNA target of the polypeptide expressed by the expression cassette.

In all embodiments, the encoded polypeptide may be a fusion protein of any embodiment or combination of embodiments herein.

In various embodiments, the kits may be used for: (1) the regulation of beneficial transgene gene expression in chimeric antigen receptor T cells, or other cells with therapeutic functions, through transcriptional regulation of synthetic enhancer or promoter sequences containing the target DNA sequences and a polypeptide of the disclosure fused with a transcriptional activation domain or a transcription repression domain; (2) fusion of a polypeptide of the disclosure with a base editing domain to permanently alter the function or expression of a gene through editing of a specific base nearby the target DNA site; (3) diversification of DNA sequence near a DNA target site through fusion of a polypeptide of the disclosure with a nickase domain and a DNA polymerase for directed evolution applications; (4) integration of genes at synthetic landing pad sites containing the target DNA sequence of a polypeptide of the disclosure fused with a nuclease domain through homology-directed DNA repair; and (5) integration of genes at synthetic landing pad sites containing designed target DNA sequences of a polypeptide of the disclosure fused with a recombinase or integrase domain. These methods are also part of the disclosed invention.

### EXAMPLES

Specific DNA-binding proteins (DBPs) play critical roles in biology and biotechnology, and there has been considerable interest in the engineering of DBPs with new or altered specificities for genome editing and other applications. The computational design of new DBPs that recognize arbitrary target sites remains an outstanding challenge. We describe a computational method for the design of small DBPs that recognize specific target sequences through interactions with bases in the major groove. We employ this method in conjunction with experimental screening to generate binders for 6 distinct DNA targets. These binders exhibit specificity closely matching the computational models for the target DNA sequences at as many as 6 base positions and affinities as low as 20-100 nM. The crystal structure of a designed DBP-target site complex is in close agreement with the design model, highlighting the accuracy of the design method. The designed DBPs function in both Escherichia coli and mammalian cells to regulate repression and activation of transcription of neighboring genes.

### Design Strategy

We reasoned that it could be possible to achieve general DNA sequence recognition using small compact proteins by sampling a wide variety of structures and binding modes to find those that are optimal for targeting specific sequences of interest. Sequence-specific DNA binding requires overcoming several challenges. First, the DNA double helix, with major and minor grooves, requires the use of scaffolds that can achieve shape complementarity with the DNA backbone while positioning specific protein residues for interactions with the DNA base edges. Second, recognition of DNA sequences involves distinguishing between the subtle changes in individual atom placements among the four bases (16-22) which alter the landscape of potential molecular contacts. Third, in contrast to designed protein-protein contacts mostly mediated by orientation-agnostic hydrophobic patches (15), the majority of accessible DNA base atoms require hydrogen bond interactions with polar sidechains for specific recognition (23). Not only are polar interactions harder to model accurately, but the longer polar sidechains have considerable conformational flexibility, making structure modeling more difficult and increasing opportunities for off-target base interactions through alternate sidechain rotamer conformations.

We began by generating libraries of small (<65 amino acid) and structurally diverse scaffolds (see Methods), and docked these against specific DNA target structures seeking to maximize the potential for specific sidechain-base interactions. To do this, we extended the RIFdock approach (15) to protein-DNA interactions (see Methods). RIFdock begins by enumerating a large and comprehensive set of disembodied sidechain interactions, called a Rotamer Interaction Field (RIF), that make favorable interactions with the desired target. We focused RIF generation on polar and nonpolar interactions with nucleotide base atoms in the major groove of the DNA target, with an emphasis on protein sidechain-DNA base hydrogen bonding interactions observed frequently in native protein-DNA complexes. Next, protein backbones were identified from the scaffold library using RIFdock that can host many of the sidechain interactions in the RIF without clashing with the target. We then used Rosetta™ combinatorial sequence design or a newly developed deep learning based approach (see below) to generate amino acid sequences for the scaffold backbones promoting folding to the scaffold structure as well as high-affinity and highly specific DNA binding.

Experimental characterization of a first round of designs generated using three-helix bundle scaffolds similar to those used for general protein binder design (15) showed that few bound their DNA targets and none bound specifically. To understand the reason for this failure, we carried out detailed structural inspection of these designs compared to natural DBPs. We observed that most natural DBPs make backbone amide-mediated hydrogen bond interactions with DNA phosphate oxygens (herein called mainchain-phosphate hydrogen bonds) that were rare or absent in our docked and designed complexes. Thus, these scaffolds were unable to overcome the first DNA specific design challenge described above. We reasoned that simultaneously satisfying the hydrogen bond requirements of the DNA backbone phosphates and the DNA bases substantially constrains viable scaffold geometries, and hence that DNA binding would require a custom scaffold library. To generate such a library, we took advantage of the vast amount of metagenome sequence data and the accuracy of deep learning based protein structure prediction. We carried out sequence searches for helix-turn-helix (HTH) DNA-binding domains (24), generated AlphaFold2™ (AF2) structure predictions (25), and filtered these based on prediction confidence (pLDDT) and TMscore to known HTH domain structures (26). This resulted in a library of ˜26,000 HTH scaffolds that finely sample different helix orientations and loop geometries. We then repeated the RIFdock process using this scaffold set, constraining the RIF DNA base-specific interactions to the HTH recognition helix, and obtained ten million distinct docks for sequence design. In contrast to the first round with the general three-helix bundle scaffolds, many of these docks make mainchain-phosphate hydrogen bonds while harboring multiple RIF base-contacting sidechains, satisfying design challenge one.

We used both Rosetta™-based sequence design and an extended version of the deep learning-based ProteinMPNN sequence design software to promote folding to the target scaffold and high affinity binding to the DNA target (see Methods). As originally described, the ProteinMPNN graphical model generates amino acid sequences purely based on protein backbone coordinates, but a recent extension to incorporate ligand and DNA atoms in the interaction graph, called LigandMPNN, enables design in the presence of specific DNA target sites. LigandMPNN recovers a higher frequency of native amino acid identities than standard Rosetta™ sequence design calculations given protein backbones in complex with specific DNA sequences. To reduce the computational cost of full sequence design on the millions of generated scaffold docks for each target site, we first repacked only the RIF sidechain residues in the context of the target to remove potential clashes between designed sidechains, as the RIF procedure does not consider interactions between sidechains explicitly. Docks for which good protein-DNA interactions could be achieved without sidechain clashes were then subjected to multiple iterations of full sequence design (2-3 minutes per scaffold with Rosetta™; ˜8 seconds per scaffold with LigandMPNN), alternating with Rosetta™ backbone relaxation to maximize complementarity to the target sequence. For both the Rosetta™ and LigandMPNN approaches we generated 200,000-300,000 designed complexes per target.

From this large set of designs for each target, we selected those with the most favorable free energy of binding (Rosetta™ ΔΔG), contact molecular surface area (15) and interface hydrogen bonds, the fewest interface buried unsatisfied hydrogen bond donors and acceptors, and with bidentate sidechain-base hydrogen bonding arrangements frequent in the Protein Data Bank (PDB) (see Methods for full details). We reasoned that specificity and affinity of designs would be improved by selecting designs with high interface sidechain preorganization, especially for long polar sidechains such as arginine and lysine, achieved through sidechain (sc)-sc hydrogen bonding and packing interactions that restrict the rotameric degrees of freedom of a given residue. To quantify the extent of preorganization, we used the Rosetta™ RotamerBoltzmann calculation (27) to estimate the probability that each sidechain making hydrogen bonds to nucleotide base atoms in the design model populates the same sidechain conformation in the apo structure. Following filtering based on the above criteria, and clustering by sequence identity, the monomeric structures of the hundreds to thousands of designs which remained for each target were predicted based on their sequences using AF2, and designs for which these were not close to the original design models were discarded. The remaining predicted monomer structures were superimposed onto the design complex by alignment on the interface residues of the original design, relaxed with Rosetta™ in the context of the DNA, and those with the most favorable DNA binding interactions as assessed with the above metrics were selected for experimental characterization. To obtain additional high-quality designs suitable for experimental characterization, the DNA interacting segments of the filtered designs were extracted, clustered, and grafted back into the original in silico scaffold library, followed by a second round of sequence design (15). We also diversified the best designs using RoseTTAFold™ Inpainting (28) focused on the resampling of scaffold loops followed by sequence design. Using a combination of these approaches, for each DNA target we generated at least 10,000 designs that passed all the structural and DNA interaction filters.

### Design Generation and Screening with Yeast Display Cell Sorting and Deep Sequencing

We created three sets of designs using variations of the overall design approach. In the first set, we generated 21,488 designs using Rosetta™-based sequence design, the motif grafting strategy, and our custom scaffold library of AF2-predicted native DNA-binding domains. In this set, the double-stranded DNA (dsDNA) targets were the DNA portions of the co-crystal structure PDBs 1BC8 (29) (9,511 designs), 1YO5 (30) (10,204 designs), and 1L3L (31) (1,773 designs). In the second design set, we generated 12,273 designs against the same DNA sequences (3,083 for 1BC8, 6,124 for 1YO5, and 3,067 for 1L3L), with the LigandMPNN sequence design strategy and the motif grafting approach for backbone resampling. In this case, rather than designing only against the dsDNA conformations found in each target's respective crystal structure, we also designed against straight B-DNA of the same sequences (6,608 designs B-form, 5,666 crystal-derived). The LigandMPNN approach was less effective at generating designs with high contact molecular surface, likely because of the ability of Rosetta™ to relax the protein backbone during sequence design, but ultimately produced designs with more favorable free energy of binding (Rosetta™ ΔΔG) and an increased number of hydrogen bonds to bases. Finally, in the third set we generated 100,000 designs using the LigandMPNN-based design pipeline and inpainting-based backbone remodeling protocol against 11 unique B-DNA targets. In all three sets, designs were filtered such that they achieved a distribution of sidechain preorganization metrics (approximated by the Rosetta™ RotamerBoltzmann metric) similar to native protein-DNA structures.

For each set of designs, synthetic oligonucleotides (230 base pairs) encoding the 50-65-residue designed proteins were ordered in a single pool and cloned into a yeast surface-expression vector. Cells containing designs that bound each DNA target were enriched by several rounds of fluorescence-activated cell sorting (FACS) using fluorescently labeled target dsDNA oligos. The naive and sorted populations for each DNA target were deep sequenced, and the frequency of each design in the starting population and after each sort was determined. From this analysis, we identified 97 designs that were substantially enriched (>100×) in pools sorted with their intended dsDNA target compared to the naive library. Of these, 44 (˜0.03% of total designs, 9 of set 1 (˜0.04%), 14 of set 2 (˜0.11%), and 21 of set 3 (˜0.02%)) had detectable binding by yeast display in a 96-well clonal screening format when labeled with 1 μM biotinylated dsDNA oligo and avidity (FIG. 3); the remainder may result from doublet transformants in the yeast pool or are very weak binders that enriched under higher dsDNA oligo concentration and avidity conditions). For each of the 44 designs, we knocked out the DNA binding interface by substituting the 2-3 residues making the most extensive interactions with the DNA bases such that the AF2 Cα RMSD was <2 Å to the original design model (table 1). These knockout mutations completely or substantially disrupted binding for all designs that had detectable binding on yeast with the original sequence (FIG. 3).

### Design Conformation and DNA-Side Footprinting of Binding Specificity

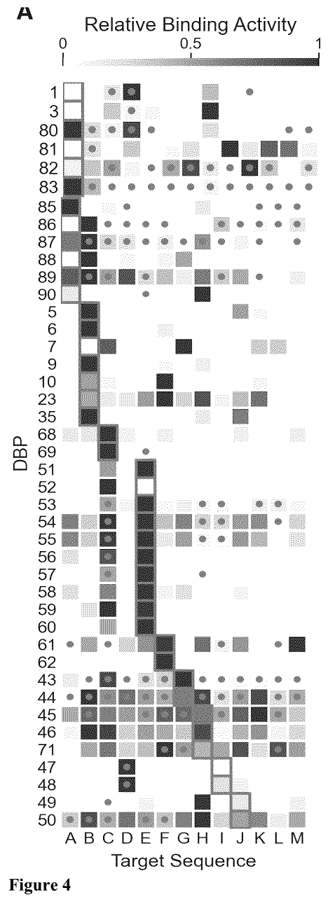

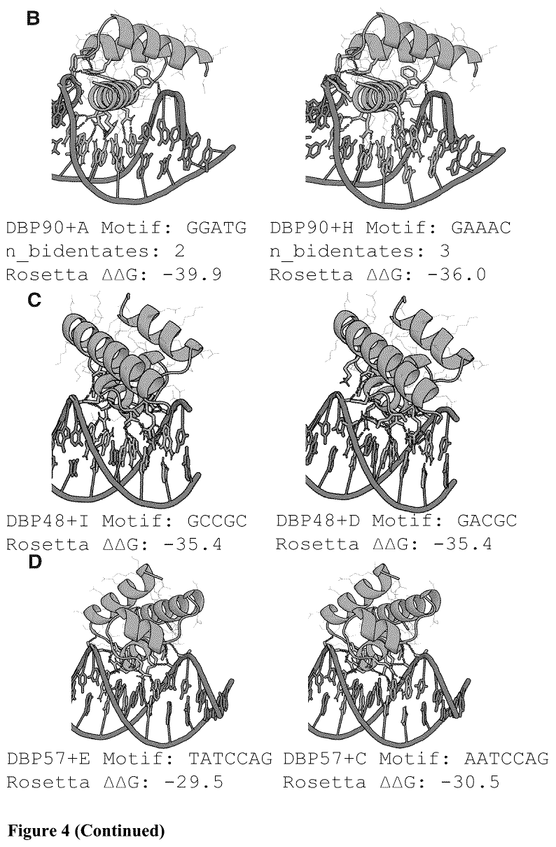

And an all-by-all screen of DBP design hits to 13 unique dsDNA targets was performed (FIG. 4, table 2). Several designs exhibited a strong preference for only their designed target sequence (e.g. DBPs 6, 9, 62), others exhibited a strong preference for 2 or 3 of the sequence targets (e.g. DBPs 1, 52, 60), and a few bound to most of the targets (e.g. DBPs 23, 44, 89). To try to understand these observed binding preferences, each tested DNA sequence was threaded onto each design complex model at all possible base pair alignments, the alternative complex models were relaxed with Rosetta™, and the model with the most favorable Rosetta™ ΔΔG was selected. We found a modest correlation between the predicted free energy of binding and the extent of off-target binding (FIG. 4); for DBPs 44 and 89, Rosetta™ ΔΔGs comparable to the original targeted sequence were obtained for most of the off target sites, consistent with the observed low specificity. Overall, we found that 14 designs bound with specificity closely consistent with the design models (DBPs 85, 5, 6, 9, 35, 43, 69, 47, 48, 51, 56, 57, 60, 62), including binders for 7 unique DNA sequences (Sequences A-G).

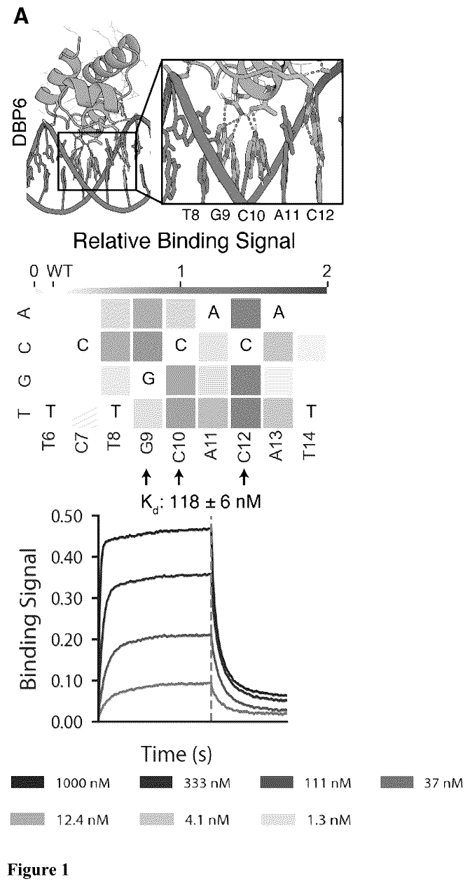

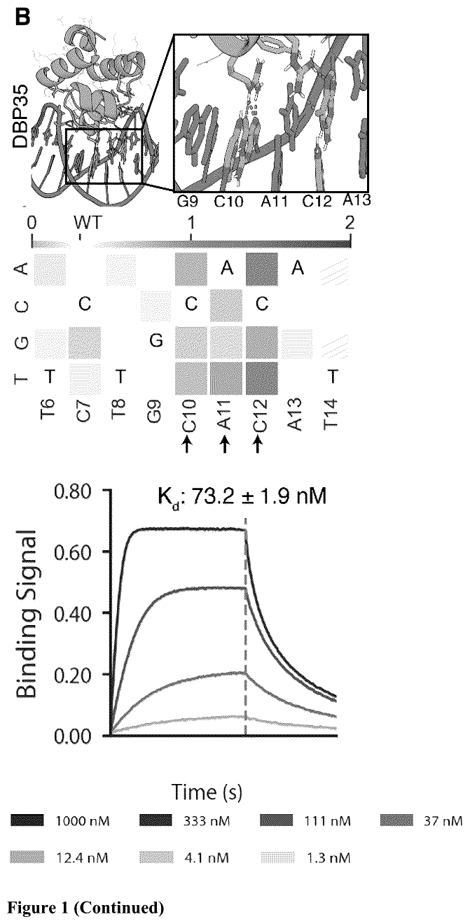

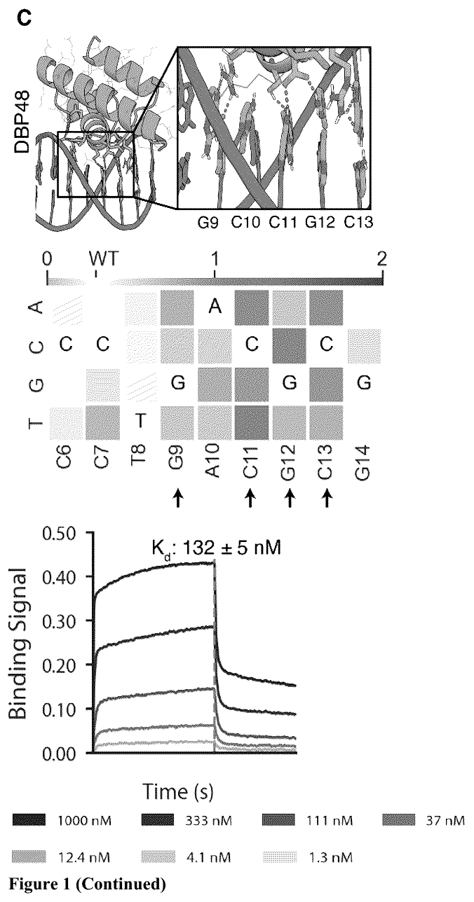

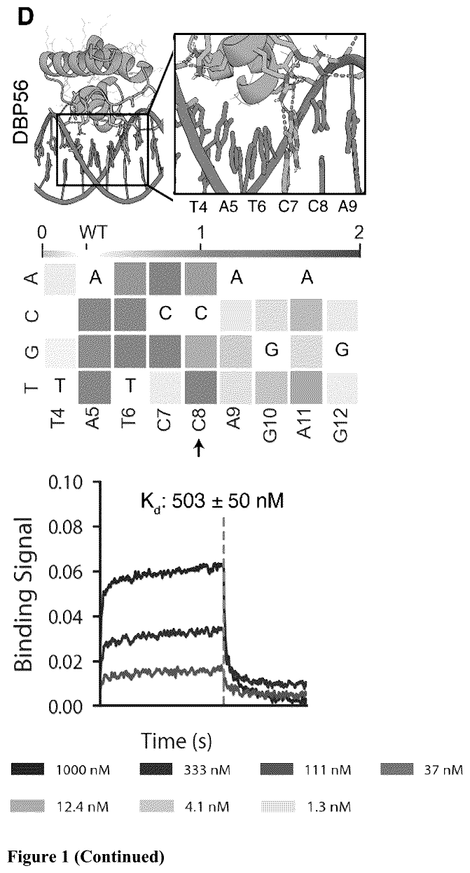

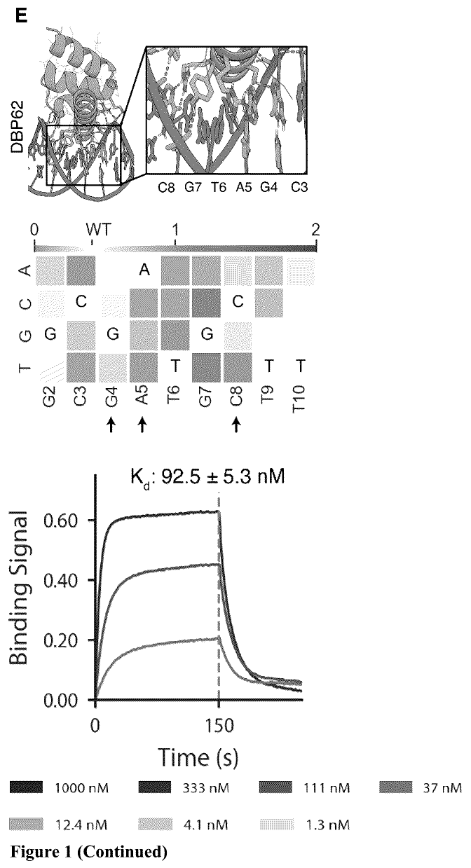

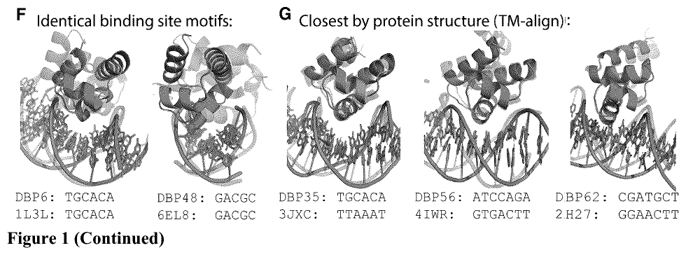

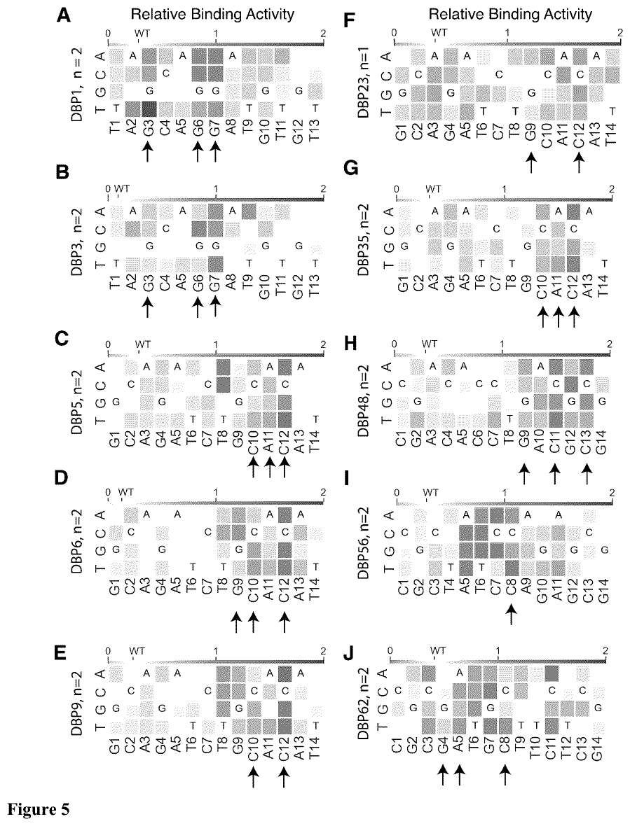

We used a yeast display competition assay to characterize the DNA binding site specificity of a subset of the designs (FIG. 1A-E, left; FIG. 5). Addition of non-biotinylated competitor dsDNA to biotinylated target sequence reduced binding signal by flow cytometry (FIG. 1A-E, middle). Scanning base substitutions through the competitor revealed positions important for binding (those at which a mutation eliminated competition of the binding signal). DBPs 1, 6, 35, 48, and 56 exhibited specificities consistent with the designed sidechain-base interactions (FIG. 1A-E). For example, in DBP6 (FIG. 1A) R31 and R36 in the design model form bidentate hydrogen bonds with the guanines of base pair positions G12 and C9, respectively, while T32 forms a hydrogen bond with C10. Substitution of the bases at positions 9, 10, and 12 eliminated competition, indicating specificity for the GCxG motif as expected (FIG. 1A). DBP62 exhibited specificity for its target site despite having relatively few base-specific hydrogen bonding interactions; specificity in this case may result from the very tightly packed interface (FIG. 1E).

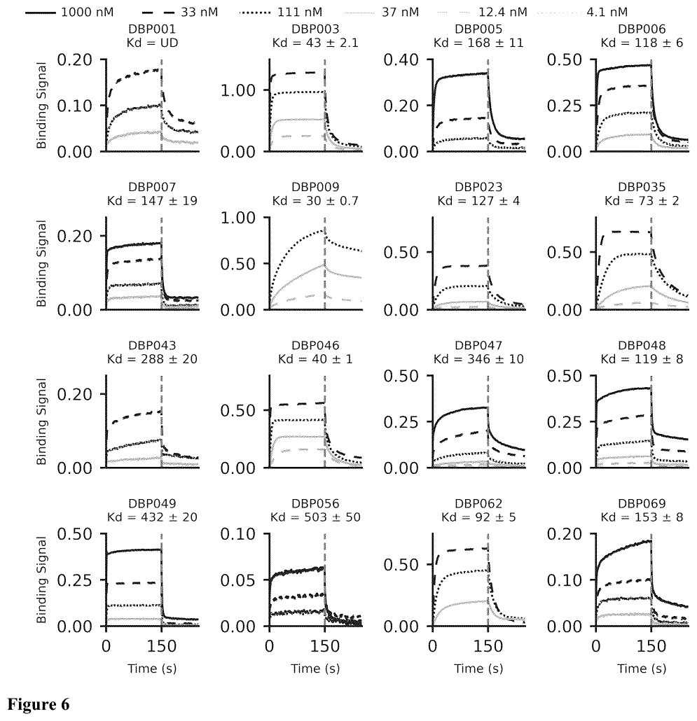

Genes encoding the same designs were encoded for E. coli expression and purified proteins were evaluated for binding in vitro. Most of the selected designs were in the soluble fraction, readily purified by Ni²⁺-NTA chromatography, and appeared monodisperse by size exclusion chromatography. Binding to the biotinylated dsDNA oligo was assessed using biolayer interferometry, and all designs were found to bind with binding affinities ranging from 20-500 nM (FIG. 1A-E (right); FIG. 6).

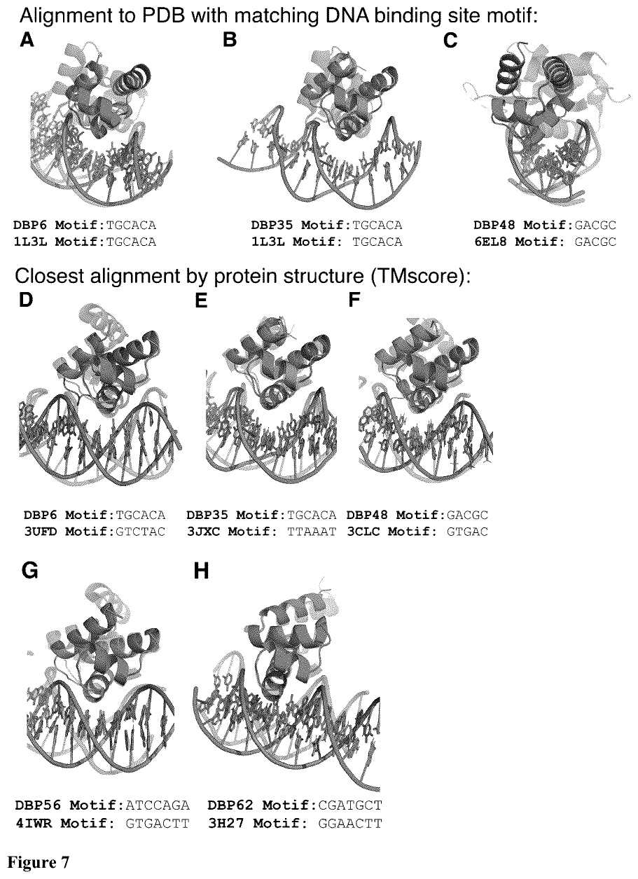

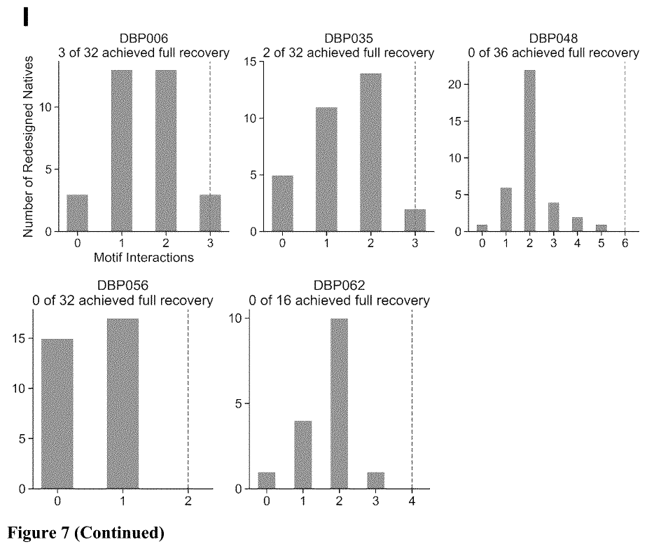

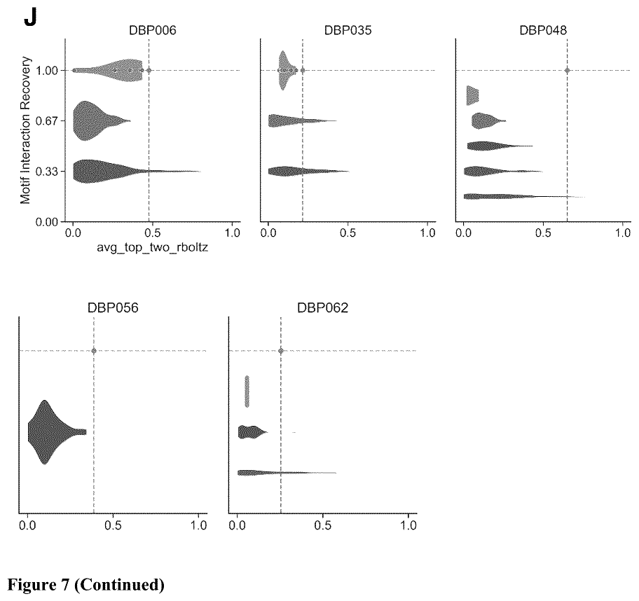

In some designs, we targeted binding towards DNA sequences found in crystal structures (e.g. DBPs 6, 35), while others were targeted to new sequences. To understand the novelty of the designed DBPs and their observed sequence preferences, we performed a comparison of the binding site motifs to co-complex structures of native DBPs in the PDB containing a protein helix in contact with bases in the DNA major groove. We found that some designs (DBPs 6, 35, 48) preferred a similar motif as native DBP structures but had substantially unique interfaces and docking orientations, while other designs (DBPs 56, 62) bound novel sequences (FIG. 1F; FIG. 7A-C). Similarly, motif searches of the JASPAR non-redundant transcription binding profile database (33, 34) revealed unique binding preferences for the same two designs compared to transcription factors with available specificity data.

Our binder design method aims to effectively sample diverse scaffold-DNA docks to find solutions optimal for binding the target DNA sequence. The method could, in principle, recover solutions similar to known native DBP-DNA complexes. To investigate this, we compared the structures of our designed DBPs to native DBP domains in DNA co-crystal structures in the PDB by TM-align (26) (closest structures: FIG. 1G; FIG. 7D-H). We found that the overall folds of the designed scaffolds had matches in the PDB, but the placement of the scaffold relative to the DNA generally differed, as expected given the de novo docking step in our approach. None of the closest matches by protein structure had more than 2 out of 6 common bases at the aligned DNA binding site positions and the sidechain-base hydrogen bond networks differed substantially. To evaluate the importance of backbone sampling through docking, we examined the ability of LigandMPNN-based sequence design to generate interfaces passing our in silico metrics when starting from crystal structures of native co-complexes rather than de novo docks. Starting from co-crystal structures with high TM-align scores to the designed DBPs, we mutated the DNA sequence in silico to the target sequence, and redesigned the sequence using LigandMPNN. We found that designs based on fixed native backbones failed to recover most of the base-specific hydrogen bonds present in the designs (FIG. 7I). In the few cases where native redesign did recover multiple base-specific hydrogen bonds, the design models scored better on sidechain preorganization by the RotamerBoltzmann metric (FIG. 7J), suggesting non-hydrogen bond features of the interface that may be critical for specific binding and require precise docking configurations. Overall, our design method is able to sample and identify designs that would not be identified through structure-based redesign of native protein-DNA co-complexes and generate specific binders for unique DNA sequences that are not known to be recognized by native proteins.

### Structural Validation by Protein-Side Footprinting of the Binding Surface

To assess the contributions of each amino acid to binding for additional designs, high-resolution footprints of the binding surface as generated by sorting site saturation mutagenesis libraries (SSMs) in which every residue was substituted with each of the 20 amino acids one at a time for DBPs 1, 6, and 35. For each of the three designs, most positions at the interface and the core were largely conserved while positions at the surface were more tolerant of substitutions. In a small number of cases, substitutions led to notable improvements in binding affinity. For DBP35, substitutions of R33 and K18 improved binding, which in the case of K18 is likely through hydrophobic contacts with the thymine methyl group at DNA position 13. In DBP6, relatively few mutations at interface positions improved binding with the exceptions of L39, D43, and 548, which may facilitate additional hydrogen bonding contacts with the phosphate backbone.

### Assessment and Optimization of Designed DBP Specificity

We explored optimization of the specificity and affinity of DBP35 by combining substitutions found in the mutational scanning. Combining R33N, which forms a potential off target interaction, K18V, which adds an additional hydrophobic interaction with the methyl stem of base pair A11, and P42Q, which potentially stabilizes the protein scaffold structure, dramatically increased binding strength observed by yeast display with detectable binding down to ˜150 pM. These mutations also increased specificity to 7 base positions as observed in a yeast competition assay (data not shown) compared with the 3 base position specificity observed in the original design (FIG. 1B), and substantially reduced binding to alternate motifs that were bound strongly in the original design. Thus, initial design hits can be optimized through limited sets of mutations to reduce binding with alternative target sequences and enhance affinity and specificity to the desired target. In this case, these optimizations contributed to strongly decreased off-target binding in the yeast display screen against the 13 DNA targets, resulting in 6 reasonably orthogonal DBP-target pairs.

### Designed DBPs Modulate Transcription in Living Cells

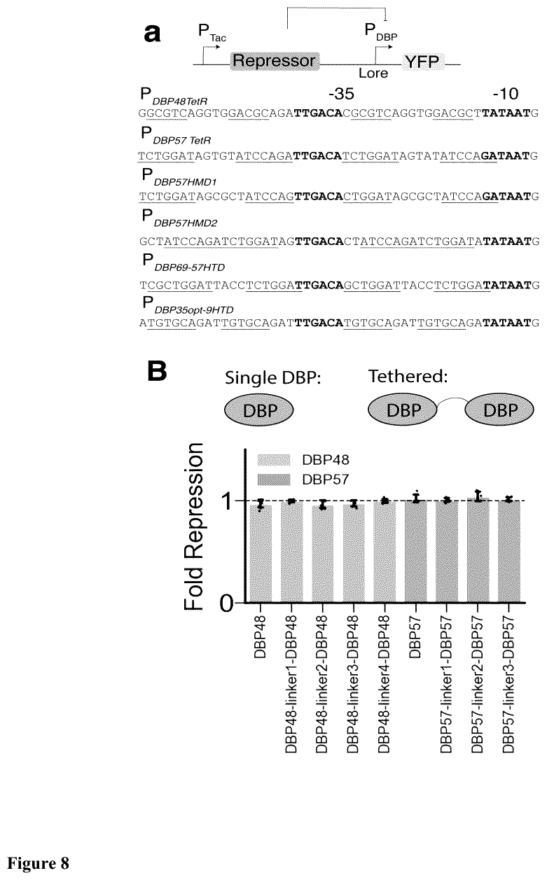

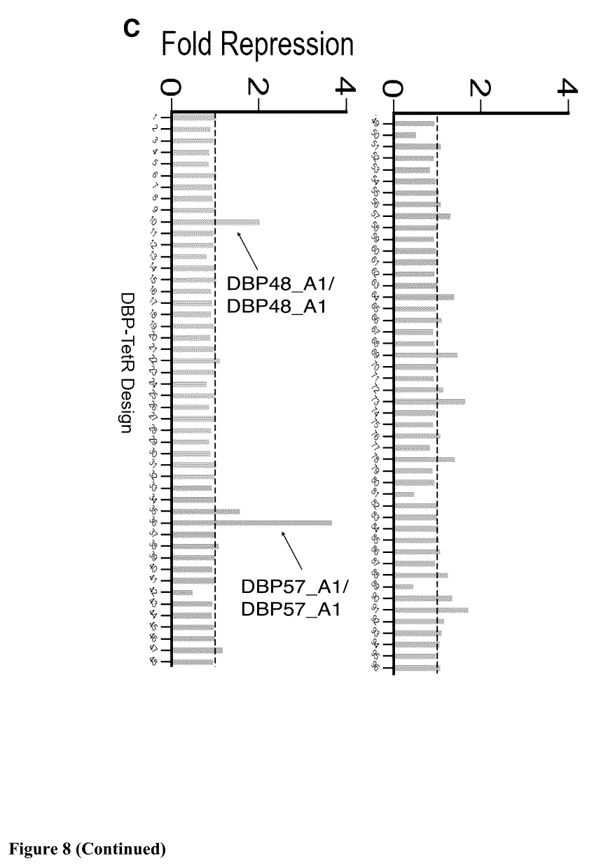

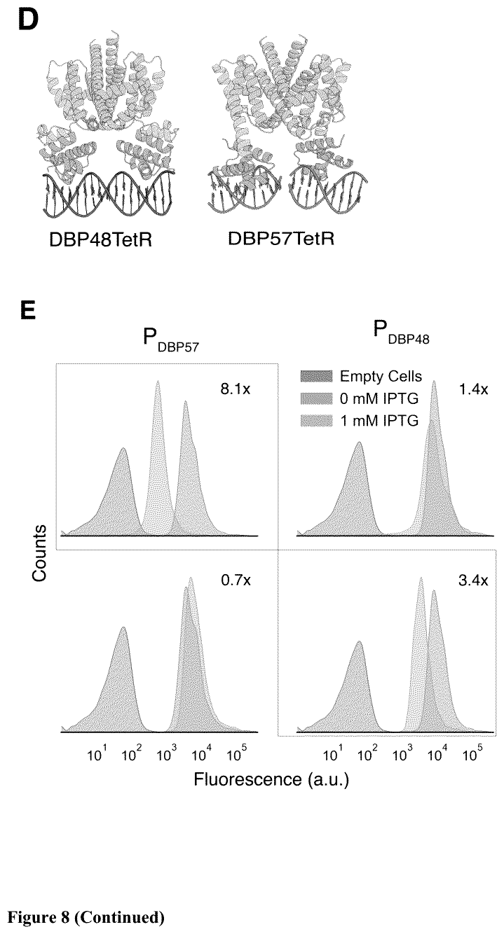

We next tested the ability of the designed DBPs to function in cells to regulate transcription. We constructed candidate NOT gates (38) to assay transcriptional repression in Escherichia coli, where the input is a designed DBP under control of the IPTG-inducible P_{Tac} promoter and the output is yellow fluorescent protein (YFP) expression driven by a promoter incorporating each DBP's DNA binding site. Single DBP domains and two copies of the same DBPs tethered through a flexible linker failed to exhibit YFP repression upon IPTG induction (FIG. 8), suggesting a need for higher affinity binding, longer sequence recognition, and/or a bulkier binding protein for effective hindrance of transcription initiation by E. coli RNA polymerase. To increase avidity and bulk, we fused the DBPs to the homodimerization domain of the TetR protein (39), using RFdiffusion (40) and ProteinMPNN sequence design to generate rigid linkers that orient the DBP domain of each dimer unit on a DNA target containing two palindromic binding sites. For DBPs 48, 69, and 57, we selected 96 TetR fusions for experimental characterization in the NOT gate circuit. Upon initial screening, we observed modest repression for several designs and selected two for further validation (one incorporating DBP48, the other DBP57) (FIG. 8). Titration of IPTG into E. coli cultures containing each circuit led to substantial titratable repression for both vectors containing cognate promoters (DBP48-TetR+P_{DBP48}, DBP57-TetR+P_{DBP57}), but no repression (DBP48-TetR+P_{DBP57}) or significantly less repression (DBP48-TetR+P_{DBP57}) for circuits with swapped promoters. The lack of repression with flexibly linked DBP domains suggest that rigid orientation of the DBP domains may be essential to achieve transcriptional repression. While the observed repression is modest relative to that obtained with native TetR (41), we expect optimization of DBP orientation could substantially improve dynamic range to generate orthogonal repressors for gene circuits (it has been estimated that up to 130 HTH transcription factors could function in one cell without crosstalk (42) but the required diversity has not been achievable through genome mining approaches (38)).

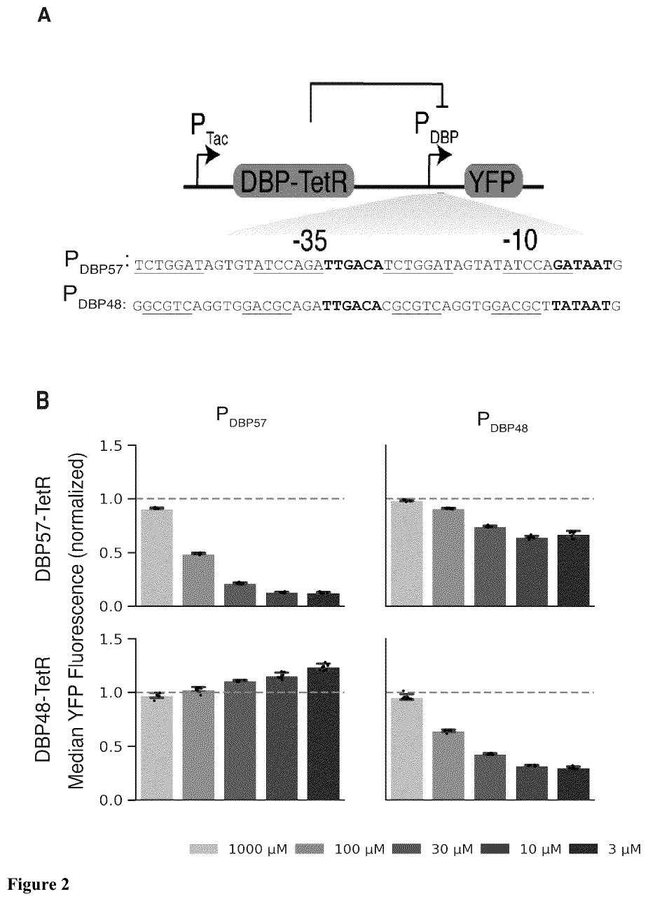

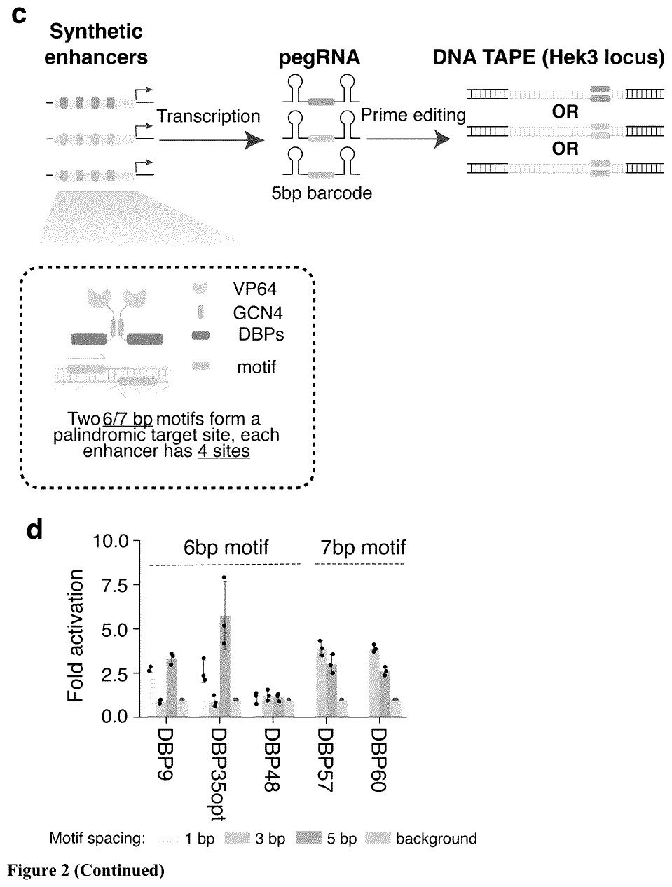

Next, we investigated whether the DBPs can be used as activators in mammalian cells. A set of synthetic transcription factors (synTFs) were created by fusing the GCN4 dimerization domain and the VP64 activation domain to the C-termini of DBPs 9, 35opt, 48, 57, and 60 which recognize 3 unique motifs. The dimerization domain allows the DBPs to recognize a palindromic target sequence consisting of two binding motifs, improving the binding affinity to the DNA sequence (FIG. 2C). We created synTF specific cis-regulatory elements (CRE) with 4 repeats spaced by 6 bp to drive the expression of downstream genes, and used the ENGRAM (43) recording technology to measure the activity of the synTFs in HEK293 cells. In ENGRAM, each CRE drives the expression of a uniquely barcoded pegRNA, which upon expression is recorded into the DNA TAPE at the HEK3 locus by prime-editor (PEmax); the activity of individual synTFs can be measured as the abundance of the barcode on the DNA TAPE (FIG. 2C). We first tested 3 different spacings-1 bp, 3 bp, and 5 bp-between the palindromic binding motifs to maximize the recorder activity. 3 synTF specific recorders and 1 TCF-LEF-recorder (negative control) were mixed with ratio 2:2:2:1 and co-transfected with synTFs into the HEK293T cells expressing PEmax. Cells were harvested and analyzed 2 days post-transfection. After analyzing the barcode abundance on the DNA TAPE, we observed 3-5 fold activation for DBP9/DBP35 and DBP57/DBP60 (FIG. 2D). Strongest activation was observed with a 5 bp spacing between two 6 bp motifs (3 bp spacing for 7 bp motif), corresponding to the 10 bp turn of the DNA helix (FIG. 2D).

### Determinants of Design Success

To assess the determinants of binding of the designed proteins, we took advantage of the large dataset (133,762 binder designs) generated in this study, 44 of which were confirmed to bind their intended target (FIG. 3) and 14 were found to preferentially bind less than 3 of the 13 tested DNA targets (FIG. 4). Across all targets, there was a strong correlation between yeast display enrichment and positive net charge of the designs, and designs that enriched with their target tended to have more sidechain- and mainchain-phosphate hydrogen bonds, higher RotamerBoltzmann probabilities of phosphate hydrogen-bonding sidechains, and low Ca RMSD to the design model. The specific binders tended to have more sidechain-involving hydrogen bonds to the phosphate backbone and lower Rosetta™ ΔΔG.

A key feature of our design method is sampling from numerous diverse starting structures and docking positions to find docks that can engage both the bases for sequence-specific recognition and the phosphate backbone to favor the designed binding mode. Similarly to the most specific designs identified, native structures appear also to strongly favor scaffolds that form mainchain-phosphate hydrogen bonds and highly pre-organized sidechain-phosphate hydrogen bonds (data not shown). To explore the importance of phosphate contacts mediating specific docks for achieving specificity for a given target site, we performed LigandMPNN redesign of 14 hits from our design campaigns against 100 randomly generated target sequences. Upon Rosetta™ relaxation of the redesigned complexes (20 LigandMPNN designed proteins per target-scaffold pair) in the presence of DNA, we observed that only 2 of the 100 sequences have as favorable Rosetta™ ΔΔGs and as many hydrogen bonds to bases, suggesting that the details of the scaffold backbone and dock make important indirect contributions to specificity by locking in the exact binding mode and narrowing the range of possible sidechain-base contacts. This makes it generally difficult to design DBPs to new DNA sequences through a native redesign approach starting from a limited set of protein-DNA backbones.

### CONCLUSION

We describe a general method for DNA binder design and demonstrate that it can generate DBPs that specifically bind arbitrary DNA sequences, including sequences that are not bound by known DBPs in the PDB. These designed DBPs function both in vitro and in living cells, as observed through transcriptional repression and activation assays in both Escherichia coli and eukaryotic cells, respectively. The method samples structurally diverse HTH scaffolds to identify complexes that can facilitate specific contacts with DNA base edges. In the best cases, generated designs were highly specific to their intended targets and specificity profiling assays strongly corroborated the design models. These results point to a promising future for de novo DBP design, where custom miniprotein scaffolds can be made to bind specific DNA sequences with high affinity. We expect that these miniproteins can be readily fused together in defined spatial orientations to allow specific targeting of longer stretches of DNA. Further, it should become possible to design oligomeric assemblies of DBPs that cooperatively bind targets with effector domains providing functionality beyond binding.

### REFERENCES

1. S. A. Wolfe, L. Nekludova, C. O. Pabo, DNA Recognition by Cys2His2 Zinc Finger Proteins. Annu. Rev. Biophys. Biomol. Struct. 29, 183-212 (2000).
2. A. Klug, The Discovery of Zinc Fingers and Their Applications in Gene Regulation and Genome Manipulation. Annu. Rev. Biochem. 79, 213-231 (2010).
3. A. M. Kabadi, C. A. Gersbach, Engineering synthetic TALE and CRISPR/Cas9 transcription factors for regulating gene expression. Methods. 69, 188-197 (2014).
4. J. K. Joung, J. D. Sander, TALENs: a widely applicable technology for targeted genome editing. Nat. Rev. Mol. Cell Biol. 14, 49-55 (2013).
5. J. Y Wang, J. A. Doudna, CRISPR technology: A decade of genome editing is only the beginning. Science. 379, eadd8643 (2023).
6. M. C. Villegas Kcam, A. J. Tsong, J. Chappell, Rational engineering of a modular bacterial CRISPR-Cas activation platform with expanded target range. Nucleic Acids Res. 49, 4793-4802 (2021).
7. M. S. Wilken, C. Ciarlo, J. Pearl, E. Schanzer, H. Liao, B. V. Biber, K. Queitsch, J. Bloom, A. Federation, R. Acosta, S. Vong, E. Otterman, D. Dunn, H. Wang, P. Zrazhevskiy, V. Nandakumar, D. Bates, R. Sandstrom, F. D. Urnov, A. Funnell, S. Green, J. A. Stamatoyannopoulos, Quantitative dialing of gene expression via precision targeting of KRAB repressor. bioRxiv (2020), doi:https://doi.org/10.1101/2020.02.19.956730.
8. J. Ashworth, J. J. Havranek, C. M. Duarte, D. Sussman, R. J. Monnat, B. L. Stoddard, D. Baker, Computational redesign of endonuclease DNA binding and cleavage specificity. Nature. 441, 656-659 (2006).
9. J. Ashworth, G. K. Taylor, J. J. Havranek, S. A. Quadri, B. L. Stoddard, D. Baker, Computational reprogramming of homing endonuclease specificity at multiple adjacent base pairs. Nucleic Acids Res. 38, 5601-5608 (2010).
10. S. B. Thyme, J. Jarjour, R. Takeuchi, J. J. Havranek, J. Ashworth, A. M. Scharenberg, B. L. Stoddard, D. Baker, Exploitation of binding energy for catalysis and design. Nature. 461, 1300-1304 (2009).
11. U. Y Ulge, D. A. Baker, R. J. Monnat, Comprehensive computational design of mCreI homing endonuclease cleavage specificity for genome engineering. Nucleic Acids Res. 39, 4330-4339 (2011).
12. X. Liu, A. T. Meger, T. G. Gillis, S. Raman, Computation-guided redesign of promoter specificity of a bacterial RNA polymerase. bioRxiv (2022), doi:https://doi.org/10.1101/2022.11.29.518332.
13. L. Milk, R. Daber, M. Lewis, Functional rules for lac repressor-operator associations and implications for protein-DNA interactions. Protein Sci. 19, 1162-1172 (2010).
14. P.-S. Huang, S. E. Boyken, D. Baker, The coming of age of de novo protein design. Nature. 537, 320-327 (2016).
15. L. Cao, B. Coventry, I. Goreshnik, B. Huang, W. Sheffler, J. S. Park, K. M. Jude, I. Marković, R. U. Kadam, K. H. G. Verschueren, K. Verstraete, S. T. R. Walsh, N. Bennett, A. Phal, A. Yang, L. Kozodoy, M. DeWitt, L. Picton, L. Miller, E.-M. Strauch, N. D. DeBouver, A. Pires, A. K. Bera, S. Halabiya, B. Hammerson, W. Yang, S. Bernard, L. Stewart, I. A. Wilson, H. Ruohola-Baker, J. Schlessinger, S. Lee, S. N. Savvides, K. C. Garcia, D. Baker, Design of protein-binding proteins from the target structure alone. Nature. 605, 551-560 (2022).
16. N. C. Seeman, J. M. Rosenberg, A. Rich, Sequence-specific recognition of double helical nucleic acids by proteins. Proc. Natl. Acad. Sci. 73, 804-808 (1976).
17. Z. Otwinowski, R. W. Schevitz, R.-G. Zhang, C. L. Lawson, A. Joachimiak, R. Q. Marmorstein, B. F. Luisi, P. B. Sigler, Crystal structure of trp represser/operator complex at atomic resolution. Nature. 335, 321-329 (1988).
18. A. Joachimiak, T. E. Haran, P. B. Sigler, Mutagenesis supports water mediated recognition in the trp repressor-operator system. EMBO J. 13, 367-372 (1994).
19. F. Rastinejad, T. Wagner, Q. Zhao, S. Khorasanizadeh, Structure of the RXR-RAR DNA-binding complex on the retinoic acid response element DR1. EMBO J. 19, 1045-1054 (2000).
20. A. K. Aggarwal, D. W. Rodgers, M. Drottar, M. Ptashne, S. C. Harrison, Recognition of a DNA Operator by the Repressor of Phage 434: A View at High Resolution. Science. 242, 899-907 (1988).
21. C. Wolberger, Y Dong, M. Ptashne, S. C. Harrison, Structure of a phage 434 Cro/DNA complex. Nature. 335, 789-795 (1988).
22. R. Rohs, X. Jin, S. M. West, R. Joshi, B. Honig, R. S. Mann, Origins of Specificity in Protein-DNA Recognition. Annu. Rev. Biochem. 79, 233-269 (2010).
23. S. A. Coulocheri, D. G. Pigis, K. A. Papavassiliou, A. G. Papavassiliou, Hydrogen bonds in protein-DNA complexes: Where geometry meets plasticity. Biochimie. 89, 1291-1303 (2007).
24. S. C. Harrison, A. K. Aggarwal, DNA recognition by proteins with the helix-turn-helix motif Annu. Rev. Biochem. 59, 933-969 (1990).
25. J. Jumper, R. Evans, A. Pritzel, T. Green, M. Figurnov, O. Ronneberger, K. Tunyasuvunakool, R. Bates, A. Z̆idek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
26. Y Zhang, TM-align: a protein structure alignment algorithm based on the TM-score. Nucleic Acids Res. 33, 2302-2309 (2005).
27. S. J. Fleishman, S. D. Khare, N. Koga, D. Baker, Restricted sidechain plasticity in the structures of native proteins and complexes: Restricted Sidechain Plasticity. Protein Sci. 20, 753-757 (2011).
28. J. Wang, S. Lisanza, D. Juergens, D. Tischer, J. L. Watson, K. M. Castro, R. Ragotte, A. Saragovi, L. F. Milles, M. Baek, I. Anishchenko, W. Yang, D. R. Hicks, M. Exposit, T. Schlichthaerle, J.-H. Chun, J. Dauparas, N. Bennett, B. I. M. Wicky, A. Muenks, F. DiMaio, B. Correia, S. Ovchinnikov, D. Baker, Scaffolding protein functional sites using deep learning. Science. 377, 387-394 (2022).
29. Y Mo, B. Vaessen, K. Johnston, R. Marmorstein, Structures of SAP-1 Bound to DNA Targets from the E74 and c-fos Promoters. Mol. Cell. 2, 201-212 (1998).
30. Y Wang, L. Feng, M. Said, S. Balderman, Z. Fayazi, Y Liu, D. Ghosh, A. M. Gulick, Analysis of the 2.0 Å Crystal Structure of the Protein-DNA Complex of the Human PDEF Ets Domain Bound to the Prostate Specific Antigen Regulatory Site. Biochemistry. 44, 7095-7106 (2005).
31. R. Zhang, K. M. Pappas, J. L. Brace, P. C. Miller, T. Oulmassov, J. M. Molyneaux, J. C. Anderson, J. K. Bashkin, S. C. Winans, A. Joachimiak, Structure of a bacterial quorum-sensing transcription factor complexed with pheromone and DNA. Nature. 417, 971-974 (2002).
32. E. W. Sayers, E. E. Bolton, J. R. Brister, K. Canese, J. Chan, D. C. Comeau, R. Connor, K. Funk, C. Kelly, S. Kim, T. Madej, A. Marchler-Bauer, C. Lanczycki, S. Lathrop, Z. Lu, F. Thibaud-Nissen, T. Murphy, L. Phan, Y Skripchenko, T. Tse, J. Wang, R. Williams, B. W. Trawick, K. D. Pruitt, S. T. Sherry, Database resources of the national center for biotechnology information. Nucleic Acids Res. 50, D20-D26 (2022).
33. J. A. Castro-Mondragon, R. Riudavets-Puig, I. Rauluseviciute, R. Berhanu Lemma, L. Turchi, R. Blanc-Mathieu, J. Lucas, P. Boddie, A. Khan, N. Manosalva Pérez, O. Fornes, T. Y Leung, A. Aguirre, F. Hammal, D. Schmelter, D. Baranasic, B. Ballester, A. Sandelin, B. Lenhard, K. Vandepoele, W. W. Wasserman, F. Parcy, A. Mathelier, JASPAR 2022: the 9th release of the open-access database of transcription factor binding profiles. Nucleic Acids Res. 50, D165-D173 (2022).
34. S. Gupta, J. A. Stamatoyannopoulos, T. L. Bailey, W. Noble, Quantifying similarity between motifs. Genome Biol. 8, R24 (2007).
35. B. Chevalier, M. Turmel, C. Lemieux, R. J. Monnat, B. L. Stoddard, Flexible DNA Target Site Recognition by Divergent Homing Endonuclease Isoschizomers I-CreI and I-MsoI. J. Mol. Biol. 329, 253-269 (2003).
36. M. F. Berger, M. L. Bulyk, Universal protein-binding microarrays for the comprehensive characterization of the DNA-binding specificities of transcription factors. Nat. Protoc. 4, 393-411 (2009).
37. M. F. Berger, A. A. Philippakis, A. M. Qureshi, F. S. He, P. W. Estep, M. L. Bulyk, Compact, universal DNA microarrays to comprehensively determine transcription-factor binding site specificities. Nat. Biotechnol. 24, 1429-1435 (2006).
38. B. C. Stanton, A. A. K. Nielsen, A. Tamsir, K. Clancy, T. Peterson, C. A. Voigt, Genomic mining of prokaryotic repressors for orthogonal logic gates. Nat. Chem. Biol. 10, 99-105 (2014).
39. W. Hillen, C. Berens, Mechanisms underlying expression of TN10 encoded tetracycline resistance. Annu. Rev. Microbiol. 48, 345-369 (1994).
40. J. L. Watson, D. Juergens, N. R. Bennett, B. L. Trippe, J. Yim, H. E. Eisenach, W. Ahern, A. J. Borst, R. J. Ragotte, L. F. Milles, B. I M. Wicky, N. Hanikel, S. J. Pellock, A. Courbet, W. Sheffler, J. Wang, P. Venkatesh, I. Sappington, S. V. Torres, A. Lauko, V. De Bortoli, E. Mathieu, R. Barzilay, T. S. Jaakkola, F. DiMaio, M. Baek, D. Baker, Broadly applicable and accurate protein design by integrating structure prediction networks and diffusion generative models. bioRxiv (2022), doi:https://doi.org/10.1101/2022.12.09.519842.
41. R. Lutz, Independent and tight regulation of transcriptional units in Escherichia coli via the LacR/O, the TetR/O and AraC/I1-I2 regulatory elements. Nucleic Acids Res. 25, 1203-1210 (1997).
42. S. Itzkovitz, T. Tlusty, U. Alon, Coding limits on the number of transcription factors. BMC Genomics. 7, 239 (2006).
43. W. Chen, J. Choi, J. F. Nathans, V. Agarwal, B. Martin, E. Nichols, A. Leith, C. Lee, J. Shendure, Multiplex genomic recording of enhancer and signal transduction activity in mammalian cells. bioRxiv (2021), doi:https://doi.org/10.1101/2021.11.05.467434.
44. R. Rohs, S. M. West, A. Sosinsky, P. Liu, R. S. Mann, B. Honig, The role of DNA shape in protein-DNA recognition. Nature. 461, 1248-1253 (2009).
45. J. S. Lamoureux, J. T. Maynes, J. N. Mark Glover, Recognition of 5′-YpG-3′ Sequences by Coupled Stacking/Hydrogen Bonding Interactions with Amino Acid Residues. J. Mol. Biol. 335, 399-408 (2004).
46. H. B. Nelson, A. Laughon, The DNA binding specificity of the Drosophila fushi tarazu protein: a possible role for DNA bending in homeodomain recognition. New Biol. 2, 171-178 (1990).
47. S.-S. Kim, J. K. Tam, A.-F. Wang, R. S. Hegde, The Structural Basis of DNA Target Discrimination by Papillomavirus E2 Proteins. J. Biol. Chem. 275, 31245-31254 (2000).
48. J. Hizver, H. Rozenberg, F. Frolow, D. Rabinovich, Z. Shakked, DNA bending by an adenine-thymine tract and its role in gene regulation. Proc. Natl. Acad. Sci. 98, 8490-8495 (2001).
49. M. Baek, R. McHugh, I. Anishchenko, D. Baker, F. DiMaio, Accurate prediction of nucleic acid and protein-nucleic acid complexes using RoseTTAFoldNA. bioRxiv (2022), doi:https://doi.org/10.1101/2022.09.09.507333.
50. N. Anand, T. Achim, Protein Structure and Sequence Generation with Equivariant Denoising Diffusion Probabilistic Models. arXiv (2022), doi:10.48550/ARXIV.2205.15019.
51. J. Ingraham, M. Baranov, Z. Costello, V. Frappier, A. Ismail, S. Tie, W. Wang, V. Xue, F. Obermeyer, A. Beam, G. Grigoryan, Illuminating protein space with a programmable generative model (2022), doi:https://doi.org/10.1101/2022.12.01.518682.
52. D. W. Rodgers, S. C. Harrison, The complex between phage 434 repressor DNA-binding domain and operator site OR3: structural differences between consensus and non-consensus half-sites. Structure. 1, 227-240 (1993).
53. M. Lewis, C. E. Bell, A closer view of the conformation of the Lac repressor bound to operator. Nat. Struct. Biol. 7, 209-214 (2000).
54. A. White, X. Ding, J. C. vanderSpek, J. R. Murphy, D. Ringe, Structure of the metal-ion-activated diphtheria toxin repressor/tox operator complex. Nature. 394, 502-506 (1998).
55. C. Wolberger, A. K. Vershon, B. Liu, A. D. Johnson, C. O. Pabo, Crystal structure of a MATa2 homeodomain-operator complex suggests a general model for homeodomain-DNA interactions. Cell. 67, 517-528 (1991).
56. M. Steinegger, M. Meier, M. Mirdita, H. Vöhringer, S. J. Haunsberger, J. Söding, HH-suite3 for fast remote homology detection and deep protein annotation. BMC Bioinformatics. 20, 473 (2019).
57. M. Mirdita, L. von den Driesch, C. Galiez, M. J. Martin, J. Söding, M. Steinegger, Uniclust databases of clustered and deeply annotated protein sequences and alignments. Nucleic Acids Res. 45, D170-D176 (2017).
58. S. R. Eddy, Anew generation of homology search tools based on probabilistic inference. Genome Inform. Int. Conf. Genome Inform. 23, 205-211 (2009).
59. I.-M. A. Chen, K. Chu, K. Palaniappan, A. Ratner, J. Huang, M. Huntemann, P. Hajek, S. J. Ritter, C. Webb, D. Wu, N. J. Varghese, T. B. K. Reddy, S. Mukherjee, G. Ovchinnikova, M. Nolan, R. Seshadri, S. Roux, A. Visel, T. Woyke, E. A. Eloe-Fadrosh, N. C. Kyrpides, N. N. Ivanova, The IMG/M data management and analysis system v.7: content updates and new features. Nucleic Acids Res. 51, D723-D732 (2023).
60. M. Steinegger, J. Söding, MMseqs2 enables sensitive protein sequence searching for the analysis of massive data sets. Nat. Biotechnol. 35, 1026-1028 (2017).
61. G. B. Kim, Y Gao, B. O. Palsson, S. Y Lee, DeepTFactor: A deep learning-based tool for the prediction of transcription factors. Proc. Natl. Acad. Sci. 118, e2021171118 (2021).
62. S. Altschul, Gapped BLAST and PSI-BLAST: a new generation of protein database search programs. Nucleic Acids Res. 25, 3389-3402 (1997).
63. K. Yamasaki, T. Akiba, T. Yamasaki, K. Harata, Structural basis for recognition of the matrix attachment region of DNA by transcription factor SATB1. Nucleic Acids Res. 35, 5073-5084 (2007).
64. J. D. Klemm, M. A. Rould, R. Aurora, W. Herr, C. O. Pabo, Crystal structure of the Oct-1 POU domain bound to an octamer site: DNA recognition with tethered DNA-binding modules. Cell. 77, 21-32 (1994).
65. M. Elrod-Erickson, T. E. Benson, C. O. Pabo, High-resolution structures of variant Zif268-DNA complexes: implications for understanding zinc finger-DNA recognition. Structure. 6, 451-464 (1998).
66. T. K. Chiu, Testing water-mediated DNA recognition by the Hin recombinase. EMBO J. 21, 801-814 (2002).
67. X.-J. Lu, 3DNA: a software package for the analysis, rebuilding and visualization of three-dimensional nucleic acid structures. Nucleic Acids Res. 31, 5108-5121 (2003).
68. C. Yanover, P. Bradley, Extensive protein and DNA backbone sampling improves structure-based specificity prediction for C2H2 zinc fingers. Nucleic Acids Res. 39, 4564-4576 (2011).
69. H. Park, P. Bradley, P. Greisen, Y Liu, V. K. Mulligan, D. E. Kim, D. Baker, F. DiMaio, Simultaneous Optimization of Biomolecular Energy Functions on Features from Small Molecules and Macromolecules. J. Chem. Theory Comput. 12, 6201-6212 (2016).
70. A. Leaver-Fay, M. Tyka, S. M. Lewis, O. F. Lange, J. Thompson, R. Jacak, K. W. Kaufman, P. D. Renfrew, C. A. Smith, W. Sheffler, I. W. Davis, S. Cooper, A. Treuille, D. J. Mandell, F. Richter, Y-E. A. Ban, S. J. Fleishman, J. E. Corn, D. E. Kim, S. Lyskov, M. Berrondo, S. Mentzer, Z. Popović, J. J. Havranek, J. Karanicolas, R. Das, J. Meiler, T. Kortemme, J. J. Gray, B. Kuhlman, D. Baker, P. Bradley, “ROSETTA3: an object-oriented software suite for the simulation and design of macromolecules” in Methods in Enzymology (Elsevier, 2011; https://linkinghub.elsevier.com/retrieve/pii/B9780123812704000196), vol. 487, pp. 545-574.
71. W. Kabsch, C. Sander, Dictionary of protein secondary structure: pattern recognition of hydrogen-bonded and geometrical features. Biopolymers. 22, 2577-2637 (1983).
72. D. M. Hoover, DNAWorks: an automated method for designing oligonucleotides for PCR-based gene synthesis. Nucleic Acids Res. 30, 43e-443 (2002).
73. L. Benatuil, J. M. Perez, J. Belk, C.-M. Hsieh, An improved yeast transformation method for the generation of very large human antibody libraries. Protein Eng. Des. Sel. 23, 155-159 (2010).
74. G. Winter, D. G. Waterman, J. M. Parkhurst, A. S. Brewster, R. J. Gildea, M. Gerstel, L. Fuentes-Montero, M. Vollmar, T. Michels-Clark, I. D. Young, N. K. Sauter, G. Evans, DIALS: implementation and evaluation of a new integration package. Acta Crystallogr. Sect. Struct. Biol. 74, 85-97 (2018).
75. A. J. McCoy, R. W. Grosse-Kunstleve, P. D. Adams, M. D. Winn, L. C. Storoni, R. J. Read, Phaser crystallographic software. J. Appl. Crystallogr. 40, 658-674 (2007).
76. D. Liebschner, P. V. Afonine, M. L. Baker, G. Bunkóczi, V. B. Chen, T. I. Croll, B. Hintze, L.-W. Hung, S. Jain, A. J. McCoy, N. W. Moriarty, R. D. Oeffner, B. K. Poon, M. G. Prisant, R. J. Read, J. S. Richardson, D. C. Richardson, M. D. Sammito, O. V. Sobolev, D. H. Stockwell, T. C. Terwilliger, A. G. Urzhumtsev, L. L. Videau, C. J. Williams, P. D. Adams, Macromolecular structure determination using X-rays, neutrons and electrons: recent developments in Phenix. Acta Crystallogr Sect. Struct. Biol. 75, 861-877 (2019).
77. P. Emsley, B. Lohkamp, W. G. Scott, K. Cowtan, Features and development of Coot. Acta Crystallogr D Biol. Crystallogr 66, 486-501 (2010).
78. C. Engler, R. Kandzia, S. Marillonnet, A One Pot, One Step, Precision Cloning Method with High Throughput Capability. PLoS ONE. 3, e3647 (2008).

### Materials and Methods

### Scaffold Library Generation

Scaffolds deposited in the PDB with structural similarity to selected template backbones (PDB IDs: 1L3L (31), 1PER (52), 1EFA (53), 1DDN (54), and 1APL (55)) were identified using TM-align (26). Amino acid sequences of identified protein scaffolds were used as seeds to generate multiple sequence alignments (MSAs) using an HHBlits (56) search of the UniRef30 database (57). Resulting MSAs were used for HMMer (58) searches of the JGI metagenome protein sequence databases (59) and the Uniref100 database (57). HMMer search results were clustered to <70% sequence identity using MMSeqs2 (60) and MSAs were generated from each clustered sequence using HHBlits. AlphaFold2™ (25) was used to predict structures for each sequence using the generated MSAs. Resulting scaffolds were filtered for high confidence AlphaFold2™ pLDDT scores, TMscore to the input backbone templates, and Rosetta™ score. Scaffolds of specific topologies were supplemented with additional AlphaFold2™-predicted structures of transcription factor sequences identified from bacterial metagenomes using DeepTF (61). PSSMs were generated for each scaffold using PSI-Blast (62) and custom code for use as constraints of Rosetta™ design. All final scaffolds are available for download.

### RIF Docking of Scaffolds onto DNA Targets

Structures of B-DNA were generated by either (1) using the DNA portion of PDB structures 1BC8 (29), 1YO5 (30), 1L3L (31), 2O4A (63), 1OCT (64), 1A1F (65), and 1JJ6(66), or (2) using the software X3DNA (67), followed by a constrained Rosetta™ relax of the DNA structure. The RIF docking method performs a high-resolution search of continuous rigid-body docking space. RIF docking comprises two steps. In the first step, ensembles of interacting discrete sidechains (referred to as ‘rotamers’) tailored to the target are generated. Polar rotamers are placed on the basis of hydrogen-bond geometry whereas apolar rotamers are generated via a docking process and filtered by an energy threshold. Rotamers were only calculated for nucleotide base atoms in the major groove of the DNA target. All the RIF rotamers are stored in ˜0.5 Å sparse binning of the six-dimensional rigid body space of their backbones, allowing extremely rapid lookup of rotamers that align with a given scaffold position. To enrich for canonical protein-DNA hydrogen bond interactions, rotamers of ARG, GLN, and ASN forming bidentate hydrogen bonds with G and A bases were extracted from the PDB, clustered by RMSD, aligned to the DNA target at all G and A positions, and added to the RIF as hotspot residues. To facilitate the next docking step, RIF rotamers are further binned at 1.0 Å, 2.0 Å, 4.0 Å, 8.0 Å and 16.0 Å resolution. In the second step, a set of scaffolds is docked into the produced rotamer ensembles, using a hierarchical branch-and-bound search strategy. Starting with the coarsest 16.0 Å resolution, an enumerative search of scaffold positions is performed: the designable scaffold backbone positions are checked against the RIF to determine whether rotamers can be placed with favorable interacting scores. All acceptable scaffold positions (up to a configurable limit, typically ten million) are ranked and promoted to the next search stage. Each promoted scaffold is split into 26 child positions in the six-dimensional rigid-body space, providing a finer sampling. The search is iterated at 8.0 Å, 4.0 Å, 2.0 Å, 1.0 Å and 0.5 Å resolutions. All RIF docks were required to utilize at least 1 hotspot residue to be saved as an output.

### Energy Function Optimization

A new version of the Rosetta™ score function was trained to better evaluate the energy of protein-DNA interfaces. Additional flexibility of the DNA duplex was incorporated into Rosetta™ rotamer optimization and gradient-based minimization modules using modifications of DNA dihedral angles (68) and the score function was optimized using the same general method as previously published (69). The weights of individual terms in the score function were optimized to reproduce the geometries of DNA crystal structures. Specifically, the distributions of pairwise atomic distances, base-stacking and base-pairing geometries, and bond torsions were considered. Additional optimization was performed on tasks related to protein-DNA complex structures. These tasks included energy ranking of perturbed crystal structures, rotamer recovery in repacking crystal structures, and sequence recovery in redesigning the protein sequence of crystal structures. An additional weight was placed on the frequency of positively charged residues at interface positions, because previous score functions tended to overestimate the strength of solvent-exposed charged interactions. Similar geometric and design tasks were included for protein structures alone. Rosetta™ score weights optimized included partial atomic charges of protein and DNA, hydrogen bond strengths, and solvation energies. The resulting score function showed improvement across nearly all tasks, with the greatest improvements found in the protein-DNA energy ranking and sequence design.

### RotamerBoltzmann Filters

The Boltzmann probability of finding a given rotamer in a specific state was evaluated using the RotamerBoltzmannWeight filter in Rosetta™ (27). The RotamerBoltzmann score is an approximation of preorganization of a given residue in the unbound state. All amino acid residues forming hydrogen bonds with DNA base or phosphate atoms were evaluated by this metric, which was calculated on the protein monomer in the unbound state. The metric was estimated by fixing neighboring sidechains and assessing the Boltzmann probability distribution on rotamers accessible by the sidechain of interest. In order to increase the likelihood of a given rotamer in the protein-DNA complex, designs with lower RotamerBoltzmann scores (a score of 0 implies the rotameric state is unpopulated and a score of 1 implies the state is the only populated state) were preferentially chosen, as known native protein-DNA crystal structures tend to contain preorganized amino acid residues.

### Rosetta™-Based Interface Sequence Design

A stripped down version of the Rosetta™ score function was used to roughly design the interface of RIF dock outputs (15). This step was primarily used to replace clashing residues before evaluating for design potential. Specifically, fa_elec, lk_ball[iso,bridge,bridge_unclp], and the_intra_terms were disabled. All that remained were Lennard-Jones, implicit solvation and backbone-dependent one-body energies (fa_dun, p_aa_pp, rama_prepro). Additionally, flags were used to limit the number of rotamers built at each position (Supplementary Information). After the rapid design step, the designs were minimized twice: once with a low-repulsive score function and again with a normal-repulsive score function. Rosetta™ ΔΔG and contact molecular surface were then calculated on the roughly designed interface. A maximum likelihood estimator was used to give each predicted design a likelihood that it should be selected to move forward. A subset of the docks to be evaluated were subjected to the full sequence design, and their final metric values calculated. With a goal threshold for each filter, each fully designed output can be marked as pass or fail for each metric independently. Then, by binning the fully designed outputs by their values from the rapid trajectory and plotting the fraction of designs that pass the goal threshold, the probability that each predicted design passes each filter can be calculated. From here, the probability of passing each filter may be multiplied together to arrive at the final probability of passing all filters. This final probability can then be used to rank the designs and pick the best designs to move forward to full sequence optimization. Note that the rapid design protocol here is used merely to rank the designs, not to optimize them; the original docks are the structures carried forward.

These docked conformations passing the rapid design protocol were further optimized to generate shape- and chemically-complementary interfaces using a Rosetta™ FastDesign protocol, alternating between sidechain rotamer optimization and gradient descent-based energy minimization. Design was performed with a sequence profile constraint based on an MSA of the originating native scaffold sequence and cross-interface interactions upweighted to maximize contacts and shape complementarity. We did not allow Rosetta™ to repack or relax the DNA target during the design procedure. A python script was implemented to automatically carry out rapid design evaluation, pre-emption, and full sequence design. Computational metrics of the final design models were calculated using Rosetta™, which includes ΔΔG, hydrogen bonds to base atoms, and contact molecular surface, among others, for design selection. All the script and flag files to run the programs are provided in the Supplementary Information. ProteinMPNN was used to redesign non-interface residues in the final design step, before AF2 monomer validation.

### LigandMPNN-Based Sequence Design

LigandMPNN was used for sequence design in the context of DNA. The network was used to optimize the protein sequence for given protein-DNA complex structures during design, whereby amino acids were determined autoregressively by the identity and location of neighboring protein and DNA residues. When the full protein sequence was determined, it was threaded onto the input protein scaffold. As in the above Rosetta™-based interface sequence design protocol, the designs were minimized with a low-repulsive score function and again with a normal-repulsive score function, and Rosetta™ ΔΔG and contact molecular surface were calculated on the roughly designed interface. A maximum likelihood estimator was used to pre-empt design of poor docks as described in the above Rosetta™-based sequence design protocol. A python script was implemented to automatically carry out MPNN sequence design, rapid design evaluation, pre-emption, and Rosetta™ Relax. Computational metrics of the final design models were calculated using Rosetta™, which includes ΔΔG, interface hydrogen bonds, and contact molecular surface, among others. LigandMPNN temperatures of 0.2-0.3 were used earlier in the design process to increase the variability of amino acid sequences, while a temperature of 0.1 was used later to determine the more probable sequences. Key residues making base-specific hydrogen bonds with DNA atoms were fixed in later stages of the pipeline to encourage the design of supporting residues. All the script and flag files to run the programs are provided in the Supplementary Information.

### Backbone Resampling with Motif Grafting

Motif grafting was performed as previously reported (15). Briefly, the binding energy and interface metrics for all the continuous secondary structure motifs (helix, strand and loop) were calculated for the designs generated in the broad search stage, as performed in previous work (15). The motifs with good interactions (based on binding energy and other interface metrics, such as contact molecular surface) with the target were extracted and aligned using the target structure as the reference. All the motifs were then clustered based on an energy-based TM-align-like clustering algorithm (26) without any further superimposition. The best motif from each cluster was then selected based on the per-position weighted Rosetta™ binding energy, using the average energy across all the aligned motifs at each position as the weight. Around 500-2,000 best motifs were selected, and the scaffold library was superimposed onto these motifs using the MotifGraft mover (70). Interface sequences were further optimized, and computational metrics were computed for the final optimized designs as described in the Rosetta™- and LigandMPNN-based sequence design methods.

### Backbone Remodeling with Protein Inpainting

Scaffold secondary structures were determined using DSSP (71). ProteinInpainting contigs were generated for each design that mask scaffold loops longer than 4 residues and surrounding residues, while ensuring that all residues forming hydrogen bonds to the DNA backbone were conserved. 10-20 unique contigs were generated for each design and sequences were constrained to a maximum of 65 amino acids. ProteinInpainting outputs were aligned to the DNA target using fixed interface residues of the input structure. The aligned ProteinInpainting outputs were subject to several further LigandMPNN+FastRelax rounds before AF2 monomer prediction and superposition steps.

### AF2 Monomer Validation and Superposition

AF2 structures were produced using the single sequence of each design. AF2 was run with model 1 and 12 recycles for each design. C-alpha RMSD of the AF2 structures to each respective design model were calculated. AF2 structures were superpositioned onto the DNA target using the backbone coordinates of interface residues within 8 Å of the DNA target. A fixed backbone Rosetta™ FastRelax was performed on each superpositioned complex and all relevant metrics were calculated on the final superpositioned design model.

### Design Filtering

Designs were filtered after each sequence design step and after superimposition of AlphaFold2™ models for those with the most favorable free energy of binding (Rosetta™ ΔΔG), contact molecular surface area (15) and interface hydrogen bonds, the fewest interface buried unsatisfied hydrogen bond donors and acceptors, and those containing bidentate sidechain-base hydrogen bonding arrangements frequent in the PDB, including bidentate interactions of ARG-G, GLN-A, and ASN-A. Designs were additionally filtered for those with a high RotamerBoltzmann score among ARG, LYS, GLN, or ASN residues forming hydrogen bonds with bases (max rboltz RKQE) and those with a high median RotamerBoltzmann (median rboltz) score of all residues forming hydrogen bonds with bases.

### DNA Library Preparation

All protein sequences were padded to 65 amino acids by adding a (GGS) n linker at the carboxy terminus of the designs to avoid the biased amplification of short DNA fragments during PCR reactions. The protein sequences were reversed translated and optimized using DNAworks™ 2.0 (72) with the Saccharomyces cerevisiae codon frequency table. Oligonucleotide pools encoding the designs were purchased from Agilent Technologies.

All libraries were amplified using Kapa HiFi™ polymerase (Kapa Biosystems) with a qPCR machine (Bio-Rad, CFX96). In detail, the libraries were first amplified in a 25 μl reaction, and the PCR reaction was terminated when the reaction reached half maximum yield to avoid overamplification. The PCR product was loaded onto a DNA agarose gel. The band with the expected size was cut out, and DNA fragments were extracted using QIAquick™ kits (Qiagen). Then, the DNA product was re-amplified as before to generate enough DNA for yeast transformation. The final PCR product was cleaned up with a QIAquick™ Clean up kit (Qiagen). For the yeast transformation step, 2-3 μg of linearized modified pETcon™ vector (pETcon3) and 6 μg of insert were transformed into the EBY100 yeast strain using a previously described protocol (73).

DNA libraries for deep sequencing were prepared using the same PCR protocol, except the first step started from yeast plasmid prepared from 5×10⁷ to 1×10⁸ cells by Zymoprep™ (Zymo Research). Illumina adapters and 6-bp pool-specific barcodes were added in the second qPCR step. Gel extraction was used to obtain the final DNA product for sequencing. All the different sorting pools were sequenced using Illumina NextSeq™ sequencing.

### Yeast Surface Display

Saccharomyces cerevisiae EBY100 strain cultures were grown in C-Trp-Ura medium supplemented with 2% (w/v) glucose. For induction of expression, yeast cells were centrifuged at 6,000 g for 1 min and resuspended in SGCAA medium supplemented with 0.2% (w/v) glucose at the cell density of 1×10⁷ cells per ml and induced at 30° C. for 16-24 h. Cells were washed with PBSF (PBS with 1% (w/v) BSA) and labeled with biotinylated targets using two labeling methods: with-avidity and without-avidity labeling. For the with-avidity method, the cells were incubated with biotinylated target, together with anti-c-Myc fluorescein isothiocyanate (FITC, Miltenyi Biotech) and streptavidin-phycoerythrin (SAPE, ThermoFisher). The concentration of SAPE in the with-avidity method was used at one-quarter of the concentration of the biotinylated targets. For the without-avidity method, the cells were first incubated with biotinylated targets, washed and secondarily labeled with SAPE and FITC.

Cell sorting of labeled yeast pools was performed using a Sony SH800S cell sorter. Libraries of designs were sorted using the with-avidity method for the first few rounds of screening to exclude weak binder candidates, followed by several without-avidity sorts with different concentrations of targets. For SSM libraries, two rounds of with-avidity sorts were applied and in the third round of screening the libraries were titrated with a series of decreasing concentrations of targets to enrich mutants with beneficial mutations.

For yeast display characterization of individual designs, including competition assays, DNA sequences encoding the proteins of interest were purchased as Integrated DNA Technologies (IDT) E-Blocks™, transformed into yeast cells, and incubated in 96 well culture plates. Labeling with biotinylated dsDNA targets and SAPE/FITC was performed in a 96 well plate format. For yeast display competition assays, labeling was performed without avidity using 1 μM biotinylated dsDNA duplex oligos and an excess of 8 μM non-biotinylated competitor dsDNA duplex oligos. As indicated in figure captions, some competition assays for higher affinity binders were carried out with lower dsDNA oligo concentrations. Flow cytometry analysis was performed with an Attune NxT™ flow cytometer with autosampler. Flow cytometry data analysis was performed using custom python code and the CytoFlow™ python package. For each individual sample, gating of the expression population was performed using the CytoFlow™ Gaussian Mixture Model and the ratio of SAPE channel intensity to FITC channel intensity (binding signal/expression signal) was calculated for all gated expression events of the sample.

### Deep Sequencing Analysis

The Pear program was used to assemble the fastq files from the deep sequencing runs. Translated, assembled reads were matched against the ordered design to determine the number of counts for each design in each pool. In each sequenced pool, binder enrichment was calculated by determining the percent of reads for each binder design in the pool and dividing this number by the same value in the naive expression sort pool. Designs were considered binders if >100-fold enrichment was observed in the last 1 μM with-avidity sort to the designed dsDNA target. For SSM libraries, apparent SC50 was estimated using the fitting procedure described in Longxing et al. (15)

### Protein Expression and Purification

DNA sequences encoding the proteins of interest were purchased as Integrated DNA Technologies (IDT) E-Blocks and incorporated into plasmids using Golden Gate™ assembly. The plasmids were then transformed into BL21(DE3) competent E. coli. The transformation reactions were used to inoculate starter cultures in 5 mL or 25 mL of “Terrific Broth” (TB), supplemented with 1% (w/v) glucose and 50 mg/L kanamycin. After shaking overnight at 37° C., the starter cultures were diluted 50-fold into 50 mL or 500 mL of TB with kanamycin. These cultures were incubated at 37° C., shaking, until the optical density (OD) reached 0.6-0.8, at which point protein expression was induced by the addition of IPTG. The cultures were then further incubated overnight at 18° C. Cells were harvested by centrifugation for 15 min at 3000 g, pellets resuspended in lysis buffer (150 mM NaCl, 20 mM Tris-HCl, 0.5 mg/mL DNAse I, 1 mM PMSF, pH 8.0), the cells lysed by sonication, and the lysate clarified by further centrifugation for 30 min at 20,000 g. The supernatant was passed through Ni-NTA resin in a gravity column, and then the resin was washed with 20 column volumes of high-salt wash buffer (2 M NaCl, 20 mM Tris-HCl, 20 mM Imidazole, pH 8.0). Either (A) the His-tagged protein was eluted with 2 column volumes of elution buffer (1 M NaCl, 20 mM Tris, 250 mM Imidazole, pH 8.0), or (B) the resin was further washed with 5 column volumes of SNAC buffer (100 mM CHES, 100 mM Acetone oxime, 100 mM NaCl, 500 mM GnCl, pH 8.6), incubated in 5 column volumes of SNAC buffer+0.2 mM NiCl₂ on an orbital shaker at room temperature overnight, and collected as the column flow-through. Whether cleaved or not, the protein was concentrated to about 1 mL and loaded in 500 μL samples onto a Cytiva Superdex™ 75 Increase 10/300 GL gel filtration column equilibrated in buffer (1 M NaCl, 20 mM Tris-HCl, pH 8.0). Fractions containing monomeric protein were pooled and concentrated to about 200 μL. Protein concentrations were estimated spectroscopically by absorbance at 280 nm. For proteins with no Trp, Tyr, or Cys residues, concentrations were approximated by Bradford reagent absorbance at 470 nm in comparison to BSA standards of known concentration.

### Biolayer Interferometry

Biolayer interferometry binding data were collected on an Octet™ R8 (Sartorius) and processed using the instrument's integrated software. Biotinylated dsDNA oligos were loaded onto streptavidin-coated biosensors (ForteBio) at 200 nM in PBS+1% BSA+0.05% Tween 20 for 6 min. Analyte proteins were diluted from concentrated stocks into the binding buffer. After baseline measurement in the binding buffer alone, the binding kinetics were monitored by dipping the biosensors in wells containing the target protein at the indicated concentration (association step) and then dipping the sensors back into baseline/buffer (dissociation). Data were analyzed and processed using ForteBio Data Analysis software v.9.0.0.14.

### RFdiffusion-Based Design of DBP-TetR Fusion Linkers

Diffusion inputs were generated by manually aligning DBP domains (DBPs 48, 57, and 69) symmetrically relative to the TetR homodimer scaffold. 10,000 RFdiffusion trajectories were run per input to generate rigid linkers between the DBP domains and the TetR homodimer scaffold. ProteinMPNN sequence design was performed on dimer diffusion outputs with tied positions between the two units and most residues of the DBP fixed, only allowing design of DBP residues nearby the newly diffused linker region. Homodimer complexes were predicted with ESMFold due to the inability of AF2 to predict the MPNN-designed TetR backbones. Predicted structures were filtered on RMSD of the predicted DBP regions to the input DBP domains and ESMFold pLDDT to select 96 designs across the three inputs.

### Transcriptional Repression Assays in E. coli

The pRF-TetR vector (38) was used for transcriptional repression assays in E. coli. A new version of this vector (pRF-BsmB1) was constructed by first removing the LuxR gene and then replacing the TetR gene, its terminator sequence, and regulated promoter with two BsmB1 cut sites such that new repressor variants and their associated promoters could be easily inserted via Golden Gate™ Assembly (78). For DBPs tethered with a flexible linker, a flexible linker was used to connect the C- and N- termini of two copies of the DBP. Synthetic promoters were designed by inserting DNA binding sites around the consensus −10 and −35 elements of the E. coli RNAP promoter. Genes encoding the single domain DBP, flexibly linked and TetR fusions were ordered as Twist™ synthetic gene fragments encoding the repressor gene, a transcriptional terminator, and an associated synthetic promoter. Gene fragments were ordered containing BsmB1 cut sites on either end to allow for assembly into the modified pRF-BsmB1 vector. Upon Golden Gate™ assembly with the BsmB1 Type II-S restriction enzyme, plasmids were transformed into NEB 5-alpha competent E. coli cells and streaked onto Luria-Burtani (LB) plates containing carbenicillin. Individual transformants were picked and verified via sanger sequencing. Sequence verified colonies were inoculated into 200 μL LB media containing carbenicillin for overnight growth in 96-well round bottom plates at 37° C. in a plate shaker. The following day, 2 μL of overnight cultures were transferred into a new plate containing 200 μL LB media containing carbenicillin and appropriate concentrations of Isopropyl β-D-1-thiogalactopyranoside (IPTG) and grown for ˜18 hrs in 96-well round bottom plates at 37° C. Flow cytometry analysis of cultures was performed with an Attune NxT™ flow cytometer with autosampler. Flow cytometry data analysis was performed using custom python code and the CytoFlow™ python package. For each individual sample, gating was performed using the single component CytoFlow™ Gaussian Mixture Model and median BL1-A channel fluorescence was determined for all gated expression events of each sample. The median BL1-A channel fluorescence value of empty cells without a pRF vector was subtracted from the median BL1-A value of each sample. For each repressor variant, fold repression was calculated as the ratio of median BL1-A channel fluorescence of the uninduced sample (background subtracted) to the median BL1-A channel fluorescence of the induced sample (background subtracted). Error bars represent standard deviation of 8 biological replicates.

### Transcriptional Activation in HEK293T Cells

HEK293T cells expressing PEmax™ were cultured in DMEM High glucose (GIBCO), supplemented with 10% Fetal Bovine Serum (Rocky Mountain Biologicals) and 1% penicillin-streptomycin (GIBCO). Cells were grown with 5% CO₂ at 37° C. 1×10⁵ cells were seeded on a 48-well plate a day before transfection. Enhancer plasmid and binder plasmid were mixed with a ratio of 2:1. Enhancer variants and background control were mixed with a ratio of 2:2:2:1. A total of 300 ng of plasmid were transfected using Lipofectamine™ 3000 (ThermoFisher, L3000015), following the manufacturer's protocol. Cells were harvested 2 days post-transfection. Genomic DNA was extracted based on the protocol described earlier (43). Briefly, cells were lysed using freshly prepared lysis buffer (10 mM Tris-HCl, pH 7.5; 0.05% SDS; 25 g/ml protease (ThermoFisher)) for each well. The genomic DNA mixture was incubated at 50° C. for 1 h, followed by an 80° C. enzyme inactivation step for 30 min. The DNA TAPE was amplified from the genomic DNA directly for next generation sequencing. Recorded information was extracted via custom analysis code. Each enhancer has a unique barcode representing its activity. Transcription activation was measured as the fold change in the barcode abundance relative to the negative control barcode. All measurements were performed in triplicates.

We claim:

1. A polypeptide comprising an amino acid sequence at least 50% identical, not including any amino acid insertions at identified insertion sites (i.e., any insertions are not considered when determining percent identity to the reference polypeptide), to the amino acid sequence selected from the group consisting of SEQ ID NO: 1-52, wherein the polypeptide is a sequence-specific DNA-binding polypeptide.

2. The polypeptide of claim 1, comprising an amino acid sequence at least 75% identical to the amino acid sequence selected from the group consisting of SEQ ID NO: 1-52.

3. The polypeptide of claim 1, comprising an amino acid sequence at least 90% identical to the amino acid sequence selected from the group consisting of SEQ ID NO: 1-52.

4. The polypeptide of claim 1, comprising an amino acid sequence at least 95% identical to the amino acid sequence selected from the group consisting of SEQ ID NO: 1-52.

5. The polypeptide of claim 1, wherein residues in bold font are conserved (i.e., identical) relative to the reference sequence.

6. The polypeptide of claim 1, wherein underlined residues are conserved relative to the reference sequence.

7. The polypeptide of claim 1, wherein the polypeptide comprises an amino acid sequence at least 50% identical to the amino acid sequence selected from the group consisting of SEQ ID NO:1, 2, 4, 15, 21-23, 25, 26, 30, 31, 34, 36, 38, 44, and 49-52.

8. The polypeptide of claim 7, wherein substitutions relative to the reference sequence are selected from residues listed in columns 2 or 3 of one of Tables 4-19.

9. The polypeptide of claim 7, wherein substitutions relative to the reference sequence are selected from residues listed in column 2 of one of Tables 4-19.

10. The polypeptide of claim 7, only conservative substitutions, or no substitutions are permitted relative to the reference sequence at interface residues identified in Table 4-19.

11. The polypeptide of claim 7, only conservative substitutions, or no substitutions are permitted relative to the reference sequence at core residues identified in Table 4-19.

12. The polypeptide of claim 1, wherein substitutions relative to the reference sequence are conservative amino acid substitutions.

13. A fusion protein, comprising:
(a) the polypeptide of claim 1; and
(b) one or more functional domains.

14. The fusion protein of claim 13, wherein the one or more functional domains is selected from the group consisting of a transcriptional effector domain, a multimerization scaffold protein, a nucleotide editing domain, a DNA methyltransferase domain, a nickase domain, a recombinase/integrase domain and a nuclease.

15. A nucleic acid encoding the polypeptide of claim 1.

16. An expression vector comprising the nucleic acid of claim 15 operatively linked to a promoter.

17. A host cell comprising the expression vector of claim 16.

18. A kit comprising:
(a) a first expression vector comprising the nucleic acid of claim 15 operatively linked to a promoter; and
(b) a second expression vector comprising a DNA target of the polypeptide expressed by the first expression vector.

19. A kit comprising:
(a) a first host cell comprising a chromosomally-integrated expression cassette comprising the nucleic acid of claim 15 operatively linked to a promoter; and
(b) a second host cell comprising a chromosomally-integrated DNA target of the polypeptide expressed by the expression cassette.


SEQUENCE LISTING (71 sequences)

SEQ ID NO: 1 - protein, 58 aa, synthetic construct
TARELEVAALIAQGRSNREIAEELNISERTVERYVRRILRKLGLRNRAQIAAWVIRRS

SEQ ID NO: 2 - protein, 58 aa, synthetic construct
TKREREVLKLIAEDYGNKEIANRLNISERTVERYIRRILRKLGLKNRAELVRYAIRHG

SEQ ID NO: 3 - protein, 63 aa, synthetic construct
GFGKAVKAKRAELGLTQAEFAERAGLSRRTIIRIEQGKVKATSTTAEKIAAALGTTVQELEQA

SEQ ID NO: 4 - protein, 56 aa, synthetic construct
DWAARAAAARRLRKERGLTQAELGELAGVSRTTVSRIELGRPDVSQASVDAVLAVL

SEQ ID NO: 5 - protein, 59 aa, synthetic construct
PLAELGKAIREARKKKGLTQEEVAKAAGVSRATVQRLELGKAKSIAPEKLAAIAKVVGL

SEQ ID NO: 6 - protein, 56 aa, synthetic construct
DWERRCAYARRARKELGLTQAELGELAGVSRTTVSRIERGKPDVSEASVEAVLAVL

SEQ ID NO: 7 - protein, 59 aa, synthetic construct
PLAEIGRAIKEARKRRGLTQAEVAEAAGVSRATVQRLELGKAKSIAPEKLAAIARVVGL

SEQ ID NO: 8 - protein, 54 aa, synthetic construct
VGEWVKRKRKEKGLTQEELAKLLGTSRATVQRIELGKKAPTPEQLERARRILEE

SEQ ID NO: 9 - protein, 59 aa, synthetic construct
PLAELGKAIKEARKRKGLTQAEVAKAAGVSRATVQRLELGKAKSIRPDKLRAILEVVGL

SEQ ID NO: 10 - protein, 59 aa, synthetic construct
PLAELGRAIREARRRRGLTQEEVARAAGVSRATVQRLELGKAKRIRPEKLAAIARVVGL

SEQ ID NO: 11 - protein, 59 aa, synthetic construct
PLAEIGKAIKEARKEKGLTQEEVAKAAGVSRATVQRLELGKAKSMRPEKLAAIAKVVGL

SEQ ID NO: 12 - protein, 65 aa, synthetic construct
DPILELLLEGEHTATELMRRLGLSYRTVRSRLRSLVRQGIIGYRHTGRVVYYVRDPERVRELMAR

SEQ ID NO: 13 - protein, 60 aa, synthetic construct
SPLVLAILEGVARGRTPAEIAKELGVSRRTVQNILQYLRRKHKLSLEELVPFARRVLAAR

SEQ ID NO: 14 - protein, 54 aa, synthetic construct
LAAEIKRLRREAGLTQRELAERMGVSRYTVQRYELGKRTPSPEELERILAALGV

SEQ ID NO: 15 - protein, 63 aa, synthetic construct
GFGRAVKEKRKELGLTQKEFAEKAGLSRRTIIRIERGYIVPPKATKEKIAKALGTSVEELEQA

SEQ ID NO: 16 - protein, 65 aa, synthetic construct
SAERHFLAVAEAGSYRRAAEILGVRRDTVRRAVLRIERKLGAPLFRREPVLTLTPLGRELYERLQ

SEQ ID NO: 17 - protein, 65 aa, synthetic construct
MEELKKLLESGDPKLQGEAVKKIREKLGLTQREFGKKIGVGQAKVSRIEAGKIKLTPELKEKILE

SEQ ID NO: 18 - protein, 60 aa, synthetic construct
KEERVLAALEAHPWSTLAEIAELTGLSRSTVSRILSRLRKEGKCDSRREGRKVRYWLVRR

SEQ ID NO: 19 - protein, 62 aa, synthetic construct
DREIEEFRREVRERMAEQGLTQADLARRSGLSRNTISRFLRGKTRPTPATVEAIRRALGLPA

SEQ ID NO: 20 - protein, 65 aa, synthetic construct
RRELSPLARARKAAGLTQRELAEKAGVGTATISRIERGRRPFSRLPPEKQERIAEILGVSVAELE

SEQ ID NO: 21 - protein, 65 aa, synthetic construct
MTPEERERAKEIGREIRELRRERGLTQRELADLLGVSRSTVSDIESGRRLPSEELLRRIREILGV

SEQ ID NO: 22 - protein, 65 aa, synthetic construct
MTPEEIAEAKRIGKEVKERRKELGLTQRELAEKLGVSRSTVSDIENGRRLPSEELLKKIKEILGV

SEQ ID NO: 23 - protein, 64 aa, synthetic construct
MEELEERILALLREEWPRGLGAAEIARRLGVPRSKVRTALRRLVAEGRVRVVRGRYSRYVAVEP

SEQ ID NO: 24 - protein, 65 aa, synthetic construct
GNPRKEKILEALCRGPRTSTEIAREIGVSTRTAAGLLQGLVRQGLARPRRRGRRVYYELADPSIC

SEQ ID NO: 25 - protein, 60 aa, synthetic construct
MEADPAVVFGRRLRAARRAKGLTQAELAERAGLGQGTISRYEKGRTLPSPEQVEKLLAAL

SEQ ID NO: 26 - protein, 60 aa, synthetic construct
RPLTPAEVFGRELRRLRRAAGLTQAELAERAGIGQGTVSRYEHGRRLPSPEEQERLLAAL

SEQ ID NO: 27 - protein, 62 aa, synthetic construct
RKLSPYERFGREIKERRKEAGLTQAELAELAGVGQATVSRIEKGEKVSPEILEKIREALEKA

SEQ ID NO: 28 - protein, 62 aa, synthetic construct
RVKTPFERFGEFVKRERAKAGLTQAELAKLAGVGQSTVSRIEKGKKCSPELREKIVKALKAV

SEQ ID NO: 29 - protein, 62 aa, synthetic construct
RKKSPLEIIGERIKKERKELGLTQAELAKLAGIGQSTVSRIEKGEKCSQRIIEKIFKALAAV

SEQ ID NO: 30 - protein, 62 aa, synthetic construct
PPPTPFEVAGARIKEERAKLGLTQAELAKVAGVGQATVSRIEKGRKCSWELIEKIFEALKKV

SEQ ID NO: 31 - protein, 62 aa, synthetic construct
MVLTPMERIGEFIKRARREAGLTQRELAELAGVGQSTVSRIEKGEKCSPELVEKILEALRKV

SEQ ID NO: 32 - protein, 64 aa, synthetic construct
AWTGEQLREFRKKLGLSQREFGELLGVGQSTVSRVEHGGELGPATRARLQARVDELVAEYKASQ

SEQ ID NO: 33 - protein, 60 aa, synthetic construct
MKELGKKIKERRKKLGLTQAQLSELSGVGQGTISRLEQGRGNPSPKILEKIEKVLKELEK

SEQ ID NO: 34 - protein, 65 aa, synthetic construct
DIEKIAKAVKELREELGLTQAEFAKKIGIGQGTLSRFEKGGVLSPKTMERLLKALEKEFGFDVKK

SEQ ID NO: 35 - protein, 64 aa, synthetic construct
GAKEKLWEFLLELAKKGLPFKLPSAEEIARRLGVRRRTVIGQLQSFVREGRIKLKRGVVYSVNE

SEQ ID NO: 36 - protein, 63 aa, synthetic construct
KEELEKLLKIIESLPKKFREVIILKFVEGLSYTEIAERLGVSRGAVYSRLRSALKKIEEALKK

SEQ ID NO: 37 - protein, 65 aa, synthetic construct
PLSGKELGELIKKYRDEKGLTQAEFAKLAGLGQGTISRLEKGVDRNGKEYHPGEEIREKVLKAIA

SEQ ID NO: 38 - protein, 56 aa, synthetic construct
MKEEGRKLKELRERLGLTQAELAEALGLGQSTISRLERGRKEISPEVWEKALALLE

SEQ ID NO: 39 - protein, 65 aa, synthetic construct
NTELLKQKIKEKGLSREEVAKKLGISRNTLTQKILGHRKFSPEQIEILKELLGLSEEEVKEIFFP

SEQ ID NO: 40 - protein, 65 aa, synthetic construct
ATAAQRWRLSPRETEVLELLINGYTNKEIASALNVSVRTVEVHIRRVLRKANVRRRVELVAKYYG

SEQ ID NO: 41 - protein, 58 aa, synthetic construct
TPREREVLNLLAQGYSNREIAERLNISEKTVKNYVRNILRKLGVRNRVEAVRWWLAVR

SEQ ID NO: 42 - protein, 60 aa, synthetic construct
NRIDSLSPREREVLRLIAQGYNNKEIAEQLNISEKTVKVHVRRILRKLNVHNRAELVNLK

SEQ ID NO: 43 - protein, 57 aa, synthetic construct
PREREILRLLAEGKNAWEIAQILNISVRTVRNHLRNAMRKLGARNRVQAVARALRLG

SEQ ID NO: 44 - protein, 63 aa, synthetic construct
TLSQLTPQEMRIARLASEGMPNREIATRLFISPRTVEWHLRRAMRKLGVRNRTQMARRIDTRL

SEQ ID NO: 45 - protein, 58 aa, synthetic construct
TKREAEVLELLSRGRSNKEIASILHISVRTVEWYIRRILRKLGVKNRVEAVRTAKAQG

SEQ ID NO: 46 - protein, 61 aa, synthetic construct
GVERLTPREKRVAHLAAQGLTNREIAEALHISPRAVENHLRRILRKLGIRRRRELPEALGE

SEQ ID NO: 47 - protein, 57 aa, synthetic construct
PREMEVLNLMAQGYNNKEIAARLGISEKTVKNHVRRILRKLGVRNRVQAVIIAQRNG

SEQ ID NO: 48 - protein, 63 aa, synthetic construct
SPAAFDKLTARELAVARLVAQGLPNREIAAALHISPRAVEAHLRKIYRKLGIRRRRELAALLA

SEQ ID NO: 49 - protein, 52 aa, synthetic construct
NKYQLSLLESAFQSNRYPDISQRATLASQTGLPERRIKIWFQNRRQRWKRKK

SEQ ID NO: 50 - protein, 63 aa, synthetic construct
GFGRAVKEKRKELGLTQVEFAEKAGLSRRTIINIERGYIVPQKATKEKIAKALGTSVEELEQA

SEQ ID NO: 51 - protein, 63 aa, synthetic construct
GFGRAVKEKRKELGLTQVEFAEKAGLSRRTIINIERGYIVPMKATKEKIAKALGTSVEELEQA

SEQ ID NO: 52 - protein, 63 aa, synthetic construct
GFGRAVKEKRKELGLTQVEFAEKAGLSRRTIIKIERGYIVPQKATKEKIAKALGTSVEELEQA

SEQ ID NO: 53 - DNA, 13 bp, synthetic construct
  source 1..13: mol_type = other DNA
tagcaggatgtgt

SEQ ID NO: 54 - DNA, 15 bp, synthetic construct
  source 1..15: mol_type = other DNA
gcagatctgcacatc

SEQ ID NO: 55 - DNA, 14 bp, synthetic construct
  source 1..14: mol_type = other DNA
cggctggattaccg

SEQ ID NO: 56 - DNA, 14 bp, synthetic construct
  source 1..14: mol_type = other DNA
cgctatccagagcg

SEQ ID NO: 57 - DNA, 14 bp, synthetic construct
  source 1..14: mol_type = other DNA
cgcgatgcttctcg

SEQ ID NO: 58 - DNA, 14 bp, synthetic construct
  source 1..14: mol_type = other DNA
cgagaacatagtcg

SEQ ID NO: 59 - DNA, 14 bp, synthetic construct
  source 1..14: mol_type = other DNA
cggggaaacgcccg

SEQ ID NO: 60 - DNA, 14 bp, synthetic construct
  source 1..14: mol_type = other DNA
cgcccaaagccgcg

SEQ ID NO: 61 - DNA, 14 bp, synthetic construct
  source 1..14: mol_type = other DNA
cggaggtaatgacg

SEQ ID NO: 62 - DNA, 57 bp, synthetic construct
  source 1..57: mol_type = other DNA
tctggatagtgtatccagattgacatctggaagtatatccagataatgagcacttcc

SEQ ID NO: 63 - DNA, 57 bp, synthetic construct
  source 1..57: mol_type = other DNA
ggcgtcaggtggacgcagattgacagcgtcaggtggacgcttataatgagcacttcc

SEQ ID NO: 64 - DNA, 49 bp, synthetic construct
  source 1..49: mol_type = other DNA
ggcgtcaggtggacgcagattgacacgcgtcaggtggacgcttataatg

SEQ ID NO: 65 - DNA, 48 bp, synthetic construct
  source 1..48: mol_type = other DNA
tctggatagtgtatccagattgacatctggaagtatatccagataatg

SEQ ID NO: 66 - DNA, 49 bp, synthetic construct
  source 1..49: mol_type = other DNA
tctggatagcgctatccagttgacactggatagcgctatccagatattg

SEQ ID NO: 67 - DNA, 49 bp, synthetic construct
  source 1..49: mol_type = other DNA
gctatccagatctggatagttgacactatccagatctggatatataatg

SEQ ID NO: 68 - DNA, 49 bp, synthetic construct
  source 1..49: mol_type = other DNA
tcgctggattacctctggattgacagctggattacctctggatataatg

SEQ ID NO: 69 - DNA, 49 bp, synthetic construct
  source 1..49: mol_type = other DNA
atgtgcagattgtgcagatttgacatgtgcagattgtgcagatataatg

SEQ ID NO: 70 - DNA, 14 bp, synthetic construct
  source 1..14: mol_type = other DNA
cgacacctgacgcg

SEQ ID NO: 71 - DNA, 14 bp, synthetic construct
  source 1..14: mol_type = other DNA
cggaggtaatgacg